(19) United States
(12) Patent Application Publication
(10) Pub. No.: US 2003/0124634 A1
(43) Pub. Date: Jul. 3, 2003

(54) Novel proteins involved in the synthesis and assembly of O-antigen in pseudomonas aeruginosa

(75) Inventors: Joseph S. Lam, Guelph (CA); Lori L. Burrows, Guelph (CA); Deborah Charter, Guelph (CA); Teresa de Kievit, Guelph (CA)

Correspondence Address: FOLEY AND LARDNER, SUITE 500, 3000 K STREET NW, WASHINGTON, DC, 20007, US

(73) Assignee: University of Guelph

(21) Appl. No.: 10/216,209

(22) Filed: Aug. 12, 2002

Related U.S. Application Data
(63) Continuation of application No. 09/352,994, filed on Jul. 13, 1999, now abandoned.
Application No. 09/352,994 is a division of application No. 08/846,762, filed on Apr. 30, 1997, now Pat. No. 5,994,072.
(60) Provisional application No. 60/016,510, filed on Apr. 30, 1996.
(60) Provisional application No. 60/039,473, filed on Feb. 27, 1997.

(51) Int. Cl. G01N 33/554; G01N 33/569; C07H 21/04; C12N 9/24; C12P 21/02; C12N 1/21; C12N 15/74

(52) U.S. Cl. 435/7.32; 435/69.1; 435/200; 435/252.3; 435/320.1; 536/23.2; 536/53

(57) ABSTRACT

Novel nucleic acid molecules encoding proteins involved in the synthesis and assembly of O-antigen in P. aeruginosa; and novel proteins encoded by the nucleic acid molecules are described. Methods are disclosed for detecting P.aeruginosa in a sample by determining the presence of the proteins or a nucleic acid molecule encoding the proteins in the sample.

### FIELD OF THE INVENTION

The invention relates to novel nucleic acid molecules encoding proteins involved in the synthesis and assembly of O-antigen in P. aeruginosa; the novel proteins encoded by the nucleic acid molecules; and, uses of the proteins and nucleic acid molecules.

### BACKGROUND OF THE INVENTION

The opportunistic pathogen P. aeruginosa remains a problem in the nosicomial infection of immunocompromised individuals. P. aeruginosa infections are particularly a problem in burn patients, people receiving medical implants, and in individuals suffering from cystic fibrosis (Fick, R. B. Jr., 1993). The organism is intrinsically resistant to many antibiotics and capable of forming biofilms which are recalcitrant to treatment. Several virulence factors have been identified in the pathogenesis of P. aeruginosa infections, including proteins such as exotoxin A, proteases, and exopolysaccharides including alginate and lipopolysaccharide (LPS). The LPS of P. aeruginosa is typical of Gram-negative bacteria, composed of lipid A-core oligosaccharide-O antigen repeating units.

P. aeruginosa is capable of coexpressing two distinct forms of LPS, designated A-band and B-band LPS, respectively. A-band LPS is a shorter, common form expressed by the majority of P. aeruginosa serotypes, and has a trisaccharide repeating unit of α-D-rhamnose linked 1→3, 1→3, 1Δ2. B-band LPS is the serotype-specific, O-antigen-containing form, and is a heteropolymer composed of di- to pentasaccharide repeats containing a wide variety of acyl sugars, amino sugars, and uronic acids. Both the A- and B-band repeating units are attached to lipid A-core, but there appear to be differences between them regarding point of attachment to and composition of the outer core region (Rivera et al., 1992).

The gene clusters for biosynthesis of core oligosaccharides/O-antigens rfb have been cloned and characterized from several bacterial species, including some from non-enteric genera such as Bordetella (Allen and Maskell, 1996), Haemophilus (Jarosik and Hansen, 1994), Neisseria (Gotschlich, 1994), Vibrio (Stroeher et al., 1992; Amor and Mutharia, 1995; Comstock et al., 1996), and Xanthamonas (Kingsley et al., 1993).

rfb clusters appear to be composed of mosaics of biosynthetic genes acquired horizontally from different sources (Reeves, 1993). Biochemical characterization of O-antigens from various species has shown that conservation of structure does not necessarily mirror conservation at the genetic level. Strains with identical O-antigens can differ significantly in their rfb clusters, while unique O-antigens can be encoded by only slightly variant rfb genes in other strains (Whitfield and Valvano, 1993).

Lightfoot and Lam were the first to report the cloning of genes involved in the expression of A-band (Lightfoot and Lam, 1991) and B-band (Lightfoot and Lam, 1993) LPS of P. aeruginosa. A recombinant cosmid clone pFV3 complemented A-band LPS synthesis in an A-band-deficient mutant, rd7513. pFV3 also mediated A-band LPS synthesis in five of the six P. aeruginosa O serotypes which lack A-band LPS. Another cosmid clone, pFV100, complemented B-band LPS synthesis in mutant ge6, which lacks B-band LPS. Physical mapping of the genes involved in A-band and B-band LPS synthesis indicated that the two gene clusters are physically distinct and are separated by more than 1.9 Mbp on the P. aeruginosa PAO1 genome. A-band LPS genes mapped between 5.75 and 5.89 Mbp (10.5 to 13.3 min), and B-band LPS genes mapped at 1.9 Mbp (near 37 min) on the 5.9-Mbp chromosome.

The structure of the P. aeruginosa O5 O-antigen has been elucidated (Knirel et al., 1988). O5 has a trisaccharide repeating unit of 2acetamido-3-acetamidino-2,3-dideoxy-D-mannuronic acid, 2,3 -diacetamido-D-mannuronic acid, and N-acetyl-D-fucosamine (FIG. 30). Serotypes O2, O16, O18, and O20 of P. aeruginosa have similar O-antigens to serotype O5, varying only in one linkage or one epimer from O5 (Knirel et al., 1988) (FIG. 30). Immunochemical cross reactions have also been demonstrated among LPS of serotypes O2, O5 and O16 by the use of monoclonal antibodies (Lam et al., 1992). The rfbA (herein also referred to as “psbL” and “wbp1”) from the O5 gene cluster has been characterized (Dasgupta and Lam, 1995). This O5 -antigen biosynthetic gene has been shown to hybridize only with chromosomal DNA from the group of five serotypes with similar O-antigens, and not with the remaining fifteen serotypes.

There are currently three pathways proposed for biosynthesis and assembly of LPS, the Rfc-dependent and Rfc-independent pathways. Rfc is the O-antigen polymerase, and appears to be required for assembly of heteropolymeric O-antigens (Mäkela and Stocker, 1984). In contrast, homopolymeric O-antigens appear to be assembled without an O-antigen polymerase (Whitfield, 1995). Rfc-dependent (or Wzy) LPS synthesis has been shown to involve at least two other gene products which act in concert with Rfc; RfbX (or Wzx), the putative flippase which translocates individual O-antigen units across the cytoplasmic membrane where they are polymerized by Rfc (or Wzy), and Rol (or Wzz), the regulator of O-antigen chain length, which determines the preferred O-antigen chain length characteristic of the individual strain or serotype (Batchelor et al., 1993; Bastin et al., 1993; Morona et al., 1994b; Dodgson et al., 1996).

### SUMMARY OF THE INVENTION

The present inventors have characterized a P. aeruginosa B-band (psb) gene cluster involved in the synthesis and assembly of B-band lipopolysaccharide i.e. O-antigen. The gene cluster is also known as and referred to herein as the wbp gene cluster.

The cluster contains two groups of genes, one of which is found in P. aeruginosa serotypes O2, O5, O16, O18, and O20, and the other is found in serotypes 01 to 020. The genes found in serotypes O2, O5, O16, O18, and O20 include the psbL gene also known as wbpL and rFA (Dasgupta and Lam, 1995), and the novel genes designated rol, psbA, psbB, psbC psbD, psbE, rfc, psbF, psbG, psbH, psbi, psbJ, and psbK (“Group I genes”), also known as and referred to herein as wzz, wbpA, wbpB, wbpC, wbpD, wbpE, wzy, wbpF, wbpG, wbpH, wbpi, wbpj, and wbpK respectively. The genes found in serotypes O1 to O20 include the novel genes psbM and psbN which are also known as and referred to herein as wbpM and wbpN respectively (“Group II genes”). The psb gene cluster also contains genes which are not involved in LPS synthesis including the genes rpsA and himD and the novel genes designated uvrB, insertion element IS407, hisH and hisF. The arrangement of the genes in the wbp gene cluster is shown in FIG. 1.

The identification and sequencing of the genes and proteins in the wbp gene cluster permits the identification of substances which affect O-antigen synthesis or assembly in P. aeruginosa. These substances may be useful in inhibiting O-antigen synthesis or assembly thereby rendering the microorganisms more susceptible to attack by host defence mechanisms.

Broadly stated the present invention relates to an isolated P. aeruginosa B-band gene cluster containing the following genes:rol (wzz), psbA (wbpA), psbB (wbpB), psbC (wbpC), psbD (wbpD), psbE (wbpE), rfc (wzy), psbF (wbpF), psbG (wbpG), psbH (wbpH), psbI (wbpI), psbJ (wbpJ), psbK (wbpK), psbL (wbpL), psbM (wbpM), and psbN (wbpN) involved in the synthesis, and assembly of lipopolysaccharide in P. aeruginosa. The terms in parenthesis correspond to other designations that have been given to these genes. The gene cluster may also contain the non-LPS gene uvrB, the insertion element IS407 (IS1209), the genes hisH and hisF involved in histidine synthesis, the gene rpsA which encodes a 30 S ribosomal subunit protein S1 and the gene himD which encodes an integration host factor.

The present invention also relates to nucleic acid molecules encoding the following proteins: (1) (a) Rol (also known as Wzz); (b) PsbA (also known as WbpA); (c) PsbB (also known as WbpB); (d) PsbC (also known as WbpC); (e) PsbD (also known as WbpD); (f) PsbE (also known as WbpE); (g) Rfc (also known as Wzy); (h) PsbF (also known as WbpF); (i) PsbG (also known as WbpG); (j) PsbI (also known as WbpI); (k) PsbJ (also known as WbpJ); (1) PsbK (also known as WbpK); (m) PsbM (also known as WbpM); (n) PsbH (also known as WbpH) or (o) PsbN (also known as WbpN), involved in P. aeruginosa O-antigen synthesis and assembly; (2) UvrB involved in ultraviolet repair; (3) HisH or HisF involved in histidine synthesis, or (4) RpsA a 30S ribosomal subunit protein S1. In addition, nucleic acid molecules are provided which contain sequences encoding two or more of the following proteins (1) (a) Rol (also known as Wzz); (b) PsbA (also known as WbpA); (c) PsbB (also known as WbpB); (d) PsbC (also known as WbpC); (e) PsbD (also known as WbpD); (f) PsbE (also known as WbpE); (g) Rfc (also known as Wzy); (h) PsbF (also known as WbpF); (i) HisH; (j) HisF; (k) PsbG (also known as WbpG); (1) PsbI (also known as WbpI); (m) PsbJ (also known as Wbpj); (n) PsbK (also known as WbpK); (o) PsbM (also known as WbpM); (p) PsbN (also known as WbpN); (q) PsbH (also known as WbpH); (r) PsbL (also known as WbpL); and (s) RpsA.

The invention also contemplates a nucleic acid molecule comprising a sequence encoding a truncation of a protein of the invention, an analog, or a homolog of a protein of the invention, or a truncation thereof.

The nucleic acid molecules of the invention may be inserted into an appropriate expression vector, i.e. a vector which contains the necessary elements for the transcription and translation of the inserted coding sequence. Accordingly, recombinant expression vectors adapted for transformation of a host cell may be constructed which comprise a nucleic acid molecule of the invention and one or more transcription and translation elements operatively linked to the nucleic acid molecule.

The recombinant expression vector may be used to prepare transformed host cells expressing a protein of the invention. Therefore, the invention further provides host cells containing a recombinant molecule of the invention.

The invention further provides a method for preparing a protein of the invention utilizing the purified and isolated nucleic acid molecules of the invention. In an embodiment a method for preparing a protein of the invention is provided comprising (a) transferring a recombinant expression vector of the invention into a host cell; (b) selecting transformed host cells from untransformed host cells; (c) culturing a selected transformed host cell under conditions which allow expression of the protein; and (d) isolating the protein.

The invention further broadly contemplates an isolated protein characterized in that it has part or all of the primary structural conformation (ie. continuous sequence of amino acid residues) of a novel protein encoded by a gene of the wbp gene cluster of the invention. In an embodiment of the invention, a purified protein is provided which has the amino acid sequence as shown in FIG. 3 or SEQ ID NO:2;, FIG. 4 or SEQ ID NO:3; FIG. 5 or SEQ ID NO:4; FIG. 6 or SEQ ID NO:5; FIG. 7 or SEQ ID NO:6; FIG. 8 or SEQ ID NO:7; FIG. 9 or SEQ ID NO:8; FIG. 10 or SEQ ID NO:9; FIG. 11 or SEQ ID NO:10; FIG. 12 or SEQ ID NO:11; FIG. 13 or SEQ ID NO:12; FIG. 14 or SEQ ID NO:13; FIG. 15 or SEQ ID NO:14; FIG. 16 or SEQ ID NO:15; FIG. 17 or SEQ ID NO:16; or, FIG. 18 or SEQ ID NO:17; FIG. 19 or SEQ.ID. No.: 18; or, FIG. 20 or SEQ.ID. No.: 19. The invention also includes truncations of the protein and analogs, homologs, and isoforms of the protein and truncations thereof.

The proteins of the invention may be conjugated with other molecules, such as proteins, to prepare fusion proteins. This may be accomplished, for example, by the synthesis of N-terminal or C-terminal fusion proteins.

The nucleic acid molecules of the invention allow those skilled in the art to construct nucleotide probes for use in the detection of nucleotide sequences in samples such as biological (e.g clinical specimens), food, or environmental samples. The nucleotide probes may also be used to detect nucleotide sequences that encode proteins related to or analogous to the proteins of the invention.

Accordingly, the invention provides a method for detecting the presence of a nucleic acid molecule having a sequence encoding a protein of the invention, comprising contacting the sample with a nucleotide probe which hybridizes with the nucleic acid molecule, to form a hybridization product under conditions which permit the formation of the hybridization product, and assaying for the hybridization product.

The invention further provides a kit for detecting the presence of a nucleic acid molecule having a sequence encoding a protein of the invention, comprising a nucleotide probe which hybridizes with the nucleic acid molecule, reagents required for hybridization of the nucleotide probe with the nucleic acid molecule, and directions for its use.

The nucleic acid molecules of the invention also permit the identification and isolation, or synthesis, of nucleotide sequences which may be used as primers to amplify a nucleic acid molecule of the invention, for example in the polymerase chain reaction (PCR).

Accordingly, the invention relates to a method of determining the presence of a nucleic acid molecule having a sequence encoding a protein of the invention in a sample, comprising treating the sample with primers which are capable of amplifying the nucleic acid molecule in an amplification reaction, preferably in a polymerase chain reaction, to form amplified sequences, under conditions which permit the formation of amplified sequences, and, assaying for amplified sequences.

The invention further relates to a kit for determining the presence of a nucleic acid molecule having a sequence encoding a protein of the invention in a sample, comprising primers which are capable of amplifying the nucleic acid molecule in an amplification reaction, preferably a polymerase chain reaction, to form amplified sequences, reagents required for amplifying the nucleic acid molecule thereof in the amplification reaction, means for assaying the amplified sequences, and directions for its use.

The invention also relates to an antibody specific for an epitope of a protein of the invention, and methods for preparing the antibodies. Antibodies specific for a protein encoded by a Group I gene can be used to detect P. aeruginosa serotypes O2, O5, O16, O18, and O20 in a sample, and antibodies specific for a protein encoded by a Group II gene can be used to detect P. aeruginosa serotypes O1 to O20 in a sample.

Therefore, the invention also relates to a method for detecting P. aeruginosa serotypes O2, O5, O16, O18, and O20 in a sample comprising contacting a sample with an antibody specific for an epitope of a protein encoded by a Group I gene which antibody is capable of being detected after it becomes bound to a protein in the sample, and assaying for antibody bound to protein in the sample, or unreacted antibody. A method is also provided for detecting P. aeruginosa serotypes O1 to O20 in a sample comprising contacting a sample with an antibody specific for an epitope of a protein encoded by a Group II gene which antibody is capable of being detected after it becomes bound to a protein in the sample, and assaying for antibody bound to protein in the sample, or unreacted antibody.

A kit for detecting P. aeruginosa serotypes in a sample comprising an antibody of the invention, preferably a monoclonal antibody and directions for its use is also provided. The kit may also contain reagents which are required for binding of the antibody to the protein in the sample.

As discussed above, the identification and sequencing of genes in the wbp gene cluster in P. aeruginosa permits the identification of substances which affect the activity of the proteins encoded by the genes in the cluster, or the expression of the proteins, thereby affecting O-antigen synthesis or assembly. These substances may be useful in rendering the microorganisms more susceptible to attack by host defence mechanisms. Accordingly, the invention provides a method for assaying for a substance that affects one or both of P. aeruginosa O-antigen synthesis or assembly comprising mixing a protein or nucleic acid molecule of the invention with a test substance which is suspected of affecting P. aeruginosa O-antigen synthesis or assembly, and determining the effect of the substance by comparing to a control.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF DRAWINGS

The invention will now be described in relation to the drawings:

FIG. 1 shows the organization of the P. aeruginosa PAO1 psb (wbp) gene cluster;

FIG. 2 shows the nucleic acid sequence of the P. aeruginosa PAO1 gene cluster (SEQ. ID. NO. 1);

FIG. 3 shows the amino acid sequence of the Rol protein of the invention (SEQ. ID NO. 2);

FIG. 4 shows the amino acid sequence of the PsbA (WbpA) protein of the invention (SEQ. ID NO. 3);

FIG. 5 shows the amino acid sequence of the PsbB (WbpB) protein of the invention (SEQ. ID NO. 4);

FIG. 6 shows the amino acid sequence of the PsbC (WbpC) protein of the invention (SEQ. ID NO. 5);

FIG. 7 shows the amino acid sequence of the PsbD (WbpD) protein of the invention (SEQ. ID NO. 6);

FIG. 8 shows the amino acid sequence of the PsbE (WbpE) protein of the invention (SEQ. ID NO. 7);

FIG. 9 shows the amino acid sequence of the Rfc (Wzy) protein of the invention (SEQ. ID NO. 8);

FIG. 10 shows the amino acid sequence of the PsbF (WbpF) protein of the invention (SEQ. ID NO. 9);

FIG. 11 shows the amino acid sequence of the HisH protein of the invention (SEQ. ID NO. 10);

FIG. 12 shows the amino acid sequence of the HisF protein of the invention (SEQ. ID NO. 11);

FIG. 13 shows the amino acid sequence of the PsbG (WbpG) protein of the invention (SEQ. ID NO. 12);

FIG. 14 shows the amino acid sequence of the PsbH (WbpH) protein of the invention (SEQ. ID NO. 13);

FIG. 15 shows the amino acid sequence of the PsbI (WbpI) protein of the invention (SEQ. ID NO. 14);

FIG. 16 shows the amino acid sequence of the PsbJ (WbpJ) protein of the invention (SEQ. ID NO. 15);

FIG. 17 shows the amino acid sequence of the PsbK (WbpK) protein of the invention (SEQ. ID NO. 16);

FIG. 18 shows the amino acid sequence of the PsbM (WbpM) protein of the invention (SEQ. ID NO. 17);

FIG. 19 shows the amino acid sequence of the PsbN (WbpN) protein of the invention (SEQ. ID NO. 18);

FIG. 20 shows the amino acid sequence of the UvrB protein of the invention (SEQ. ID NO. 19);

FIG. 21 shows the amino acid sequence of PsbL (SEQ. ID NO. 20) (WbpL);

FIG. 22 shows a silver-stained SDS-PAGE gel of LPS from PAO1, AK14O1, AK14O1(pFV100), and AK14O1(pFV.TK8) (Panel A) and Western immunoblots of this LPS reacted with O5-specific MAb MF15-4 (Panel B);

FIG. 23 shows restriction maps of the chromosomal inserts from pFV100 and several pFV subclones, and the results of complementation studies of the SR mutants AK14O1 and rd7513 with the pFV subclones are also shown;

FIG. 24 shows a Southern analysis of the three rfc (wzy) chromosomal mutants, OP5.2, OP5.3, and OP5.5, showing the insertion of an 875 bp Gm^{R} cassette into the rfc (wzy) gene (panel C), and restriction maps of the PAO1 wild-type (panel A) and mutant (panel B) rfc (wzy) coding regions are shown;

FIG. 25 shows a silver-stained SDS-PAGE gel (panel A) and Western blots of LPS from PAO1, AK14O1 and the three rfc (wzy) chromosomal mutants, OP5.2, OP5.3, and OP5.5 (Panels B and C); and

FIG. 26 shows the restriction maps of recombinant plasmids pFV161, pFV401, and pFV402;

FIG. 27 are blots of Southern hybridizations of chromosomal DNA from PAO1 (lane 2) and rol (wzz) mutants (lanes 3 and 4);

FIG. 28 are Western immunoblots showing the characterization of LPS from PAO1 and PAO1 rol (wzz) chromosomal mutants;

FIG. 29 is an autoradiogram showing ³⁵S-labeled proteins expressed by pFV401, which contains the rol (wzz) gene and corresponding control plasmid vector pBluescript II SK in E. coli JM 109DE3 by use of the T7 expression system;

FIG. 30 is a diagram showing the structures of the O-antigens of P. aeruginosa serotypes related to O5;

FIG. 31 shows E. coli σ⁷⁰ and similar regions in psbA (wpbA), hisH, psbG (wpbG) IS407 and psbN (wpbN);

FIG. 32 shows features of the psb genes of the psb gene cluster identifying the presumed start codon and spaces between RBS (ribosome binding sequence) and the first codon;

FIG. 33 shows the sequences of the NAD-binding domains of PsbA, PsbK and PsbM aligned with those of other bacterial proteins involved in polysaccharide biosynthesis;

FIG. 34 shows a sequence alignment for PsbA (WpbA) E. coli RffD, and B. solanaceraeum EpsD.

FIG. 35 shows a sequence alignment for PsbD (WpbD) and Bordetella pertussis BplB, CysE of a number of bacteria;

FIG. 36 shows a sequence alignment for PsbE (WpbE), and BP-BplC, BS-DegT, SEryC1, SDnrj, and BS-SpsC;

FIG. 37 shows a hydropathy index computation for sequence PsbF;

FIG. 38 shows a sequence alignment for PA-PsbI, BP-BplD, EC-NfrC, BS-OrfX, and SB-RfbC;

FIG. 39 shows a sequence alignment for PA-PsbJ BP-BplE, and YE-TrsE;

FIG. 40 shows a sequence alignment for PA-PsbL, YE-TrsF and HI-Rfe;

FIG. 41 shows a sequence alignment for PsbM, TrsG, BP-BplL, and SA-CapD;

FIG. 42 shows the nucleotide sequence of the rol (wzz) gene;

FIG. 43 is a physical map of the 5′ end of the wbp cluster;

FIG. 44 is a comparison of hydropathy plots of selected Wzz-like proteins;

FIG. 45 shows the expression of P. aeruginosa Wzz in vitro;

FIG. 46A shows an SDS-PAGE gel of LPS from Wzz knockout mutants;

FIG. 46B shows a western immunoblot using Mab 18-19;

FIG. 46C shows a western immunoblot using Mab MF154;

FIG. 47 shows the ability of P. aeruginosa O5 Wzz to function in E. Coli;

FIG. 48 shows an SDS-PAGE gel from WbpF knockout mutants;

FIG. 49 shows the amino acid and nucleotide sequence encoding Rps A; and

FIG. 50 shows the amino acid and nucleotide sequence encoding Him D.

### DETAILED DESCRIPTION OF THE INVENTION

The following standard abbreviations for the amino acid residues are used throughout the specification: A, Ala—alanine; C, Cys—cysteine; D, Asp- aspartic acid; E, Glu—glutamic acid; F, Phe—phenylalanine; G, Gly—glycine; H, His—histidine; I, Ile—isoleucine; K, Lys—lysine; L, Leu—leucine; M, Met—methionine; N, Asn—asparagine; P, Pro—proline; Q, Gln—glutamine; R, Arg—arginine; S, Ser—serine; T, Thr—threonine; V, Val—valine; W, Trp- tryptophan; Y, Tyr—tyrosine; and p.Y., P.Tyr—phosphotyrosine.

I. Nucleic Acid Molecules of the Invention

As hereinbefore mentioned, the present invention relates to an isolated P. aeruginosa B-band gene cluster containing genes involved in the synthesis and assembly of O-antigen in P. aeruginosa. The present invention also relates to the isolated genes which comprise the cluster.

The term “isolated” refers to a nucleic acid substantially free of cellular material or culture medium when produced by recombinant DNA techniques, or chemical precursors, or other chemicals when chemically synthesized. The term “nucleic acid” is intended to include DNA and RNA and can be either double stranded or single stranded.

The P. aeruginosa B-band gene cluster comprises the following genes: rol (wzz), psbA (wbpA), psbB (wbpB), psbC (wbpC), psbD (wbpD), psbE (wbpE), rfc (wzy), psbF (wbpF), psbG (wbpG), psbH (wbpH), psbI (wbpI), psbj (wbpj), psbK (wbpK), psbL (wbpL), psbM (wbpM), and psbN (wbpN) involved in the synthesis, and assembly of lipopolysaccharide in P. aeruginosa. The gene cluster may also contain the non-LPS genes hisH, hisF, himD, rspa, uvrB, and the insertion element IS407 (IS1209).

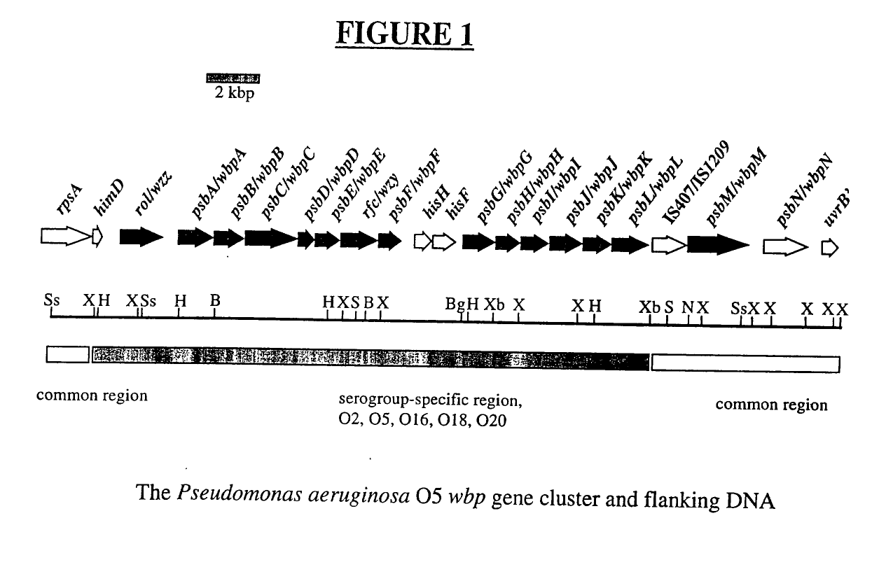

The genes preferably have the organization as shown in FIG. 1 (SEQ. ID. NO. 1). In FIG. 1, the genes necessary for sugar biosynthesis (Man(2NAc3N)A and Man(2NAc3NAc) biosynthesis) are scattered throughout the gene cluster (wpbI (psbI), wpbE (psbE), wpbD (psbD), wpbB (psbB), wpbC (psbC). The genes encoding transferases are interspersed throughout the wpb (psb) cluster (wpbH (psbH), wpbJ (psbJ), wpbL, (wpbL)), and are separated from one another by one gene each. The gene encoding the putative first transferase (Wpb (PsbL)), thought to initiate O-antigen assembly by attachment of an FucNAc residue to undecaprenol, is the most distal.

The invention provides nucleic acid molecules encoding the following proteins: (1) (a) Rol (Wzz); (b) PsbA (WbpA); (c) PsbB (WbpB); (d) PsbC (WbpC); (e) PsbD (WbpD); (f) PsbE (WbpE); (g) Rfc (Wzy); (h) PsbF (WbpF); (i) PsbG (WbpG); (j) PsbI (WbpI); (k) PsbJ (WbpJ); (1) PsbK (WbpK); (m) PsbM (WbpM); (n) PsbH (WbpH); and (o) PsbN (WbpN) involved in P. aeruginosa O-antigen synthesis and assembly; (2) UvrB involved in ultraviolet repair; (3) HisH or HisF involved in histidine synthesis or (4) himD involved in host factor integration and (5) RpsA a 30S ribosomal subunit protein S1. In addition, nucleic acid molecules are provided which contain sequences encoding two or more of the following proteins (1) (a) Rol (wzz); (b) PsbA (WbpA); (c) PsbB (WbpB); (d) PsbC (WbpC); (e) PsbD (WbpD); (f) PsbE (WbpE); (g) Rfc (Wzy); (h) PsbF (WbpF); (i) HisH; (j) HisF; (k) PsbG (WbpG); (l) PsbI (WbpI); (m) PsbJ (WbpJ); (n) PsbK (WbpK); (o) PsbM (WbpM); (p) PsbN (WbpN); (q) PsbH (WbpH); (r) PsbL (WbpL); (s) RpsA or (t) HimD.

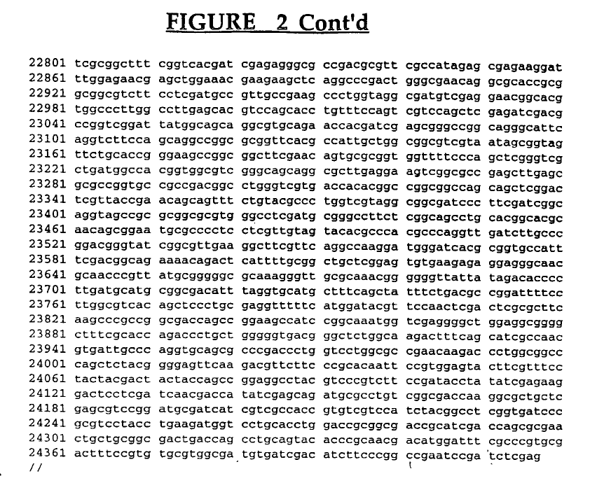

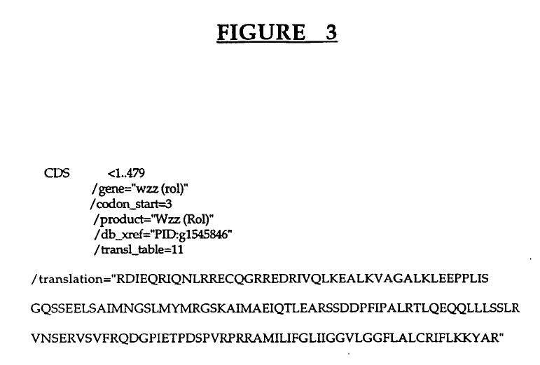

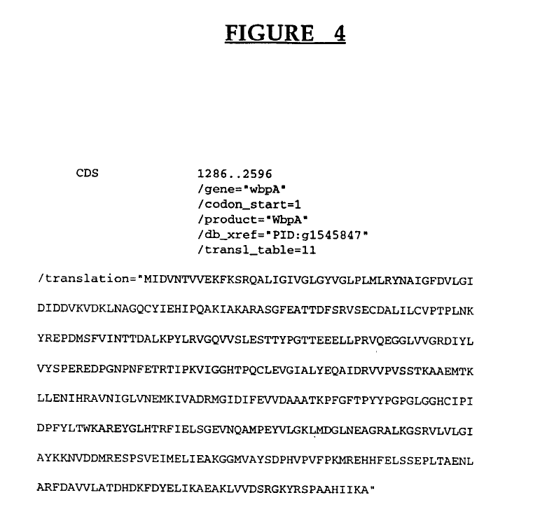

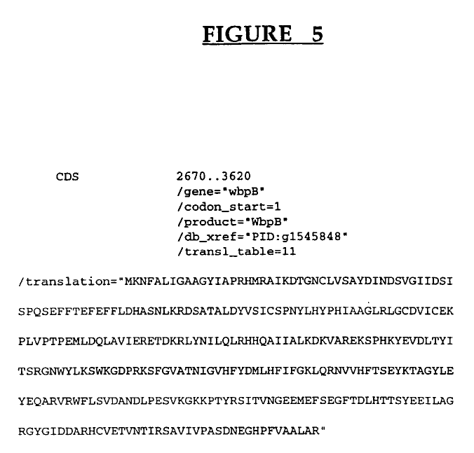

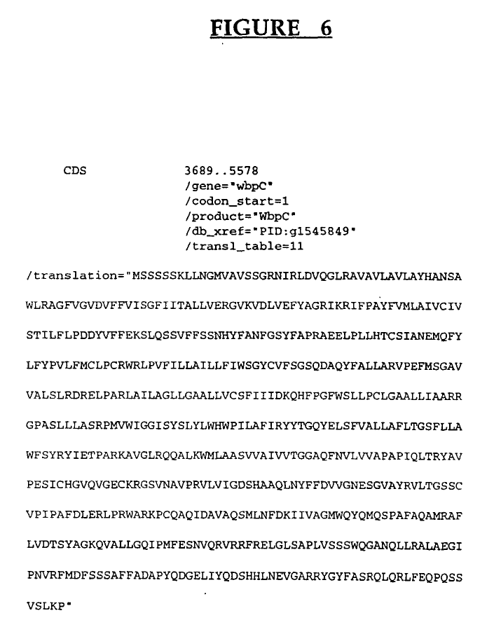

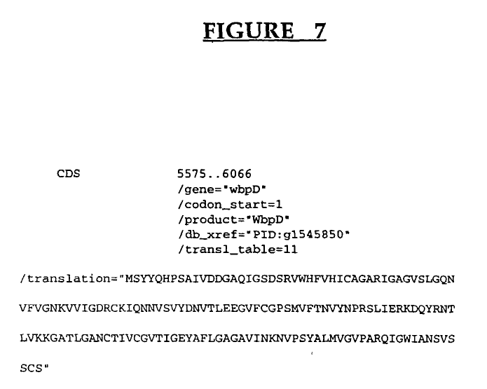

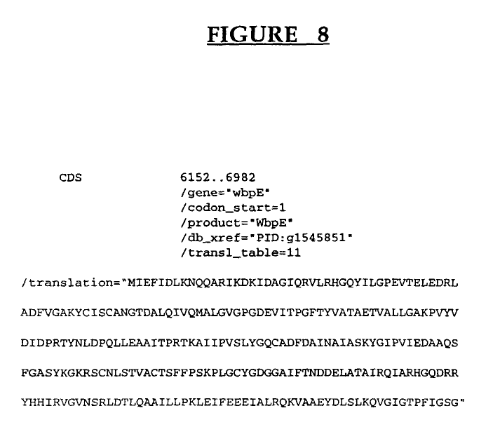

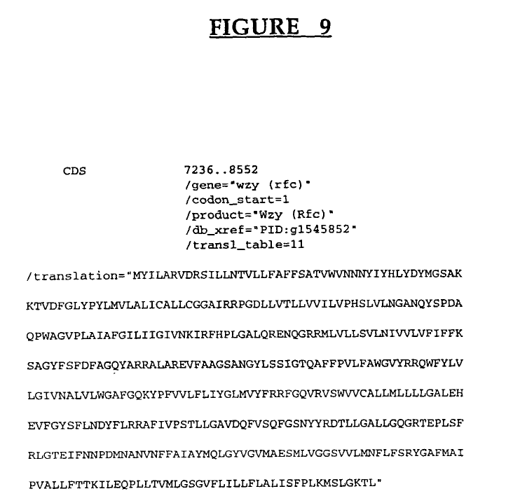

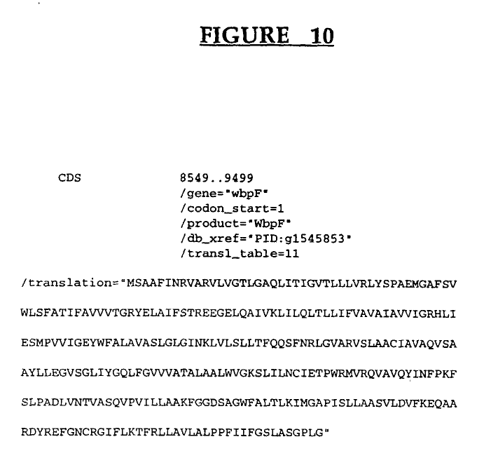

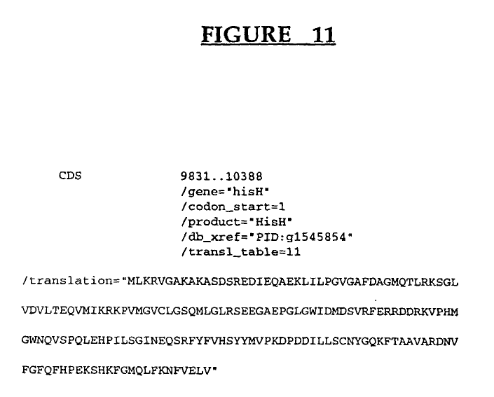

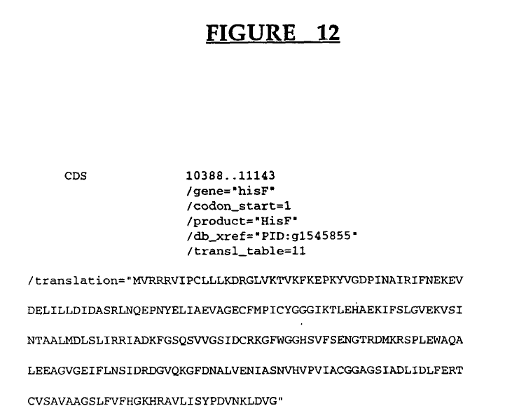

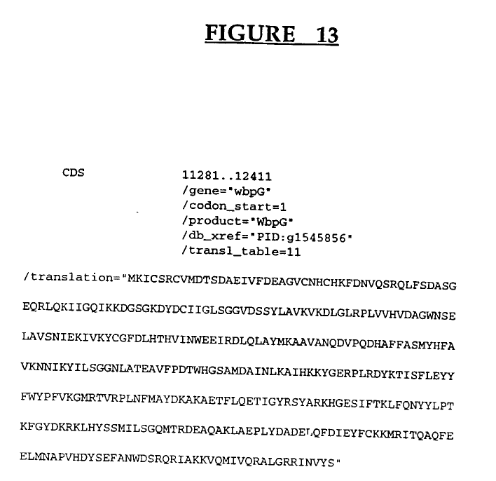

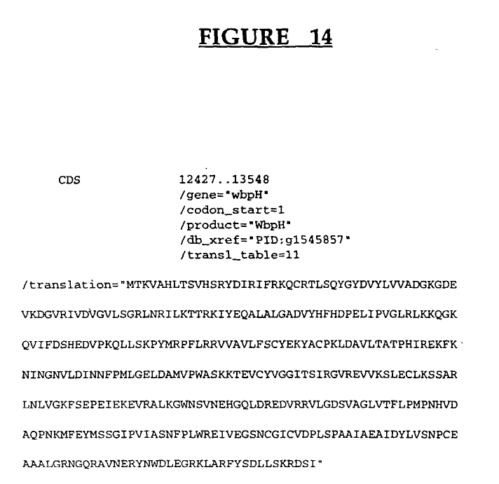

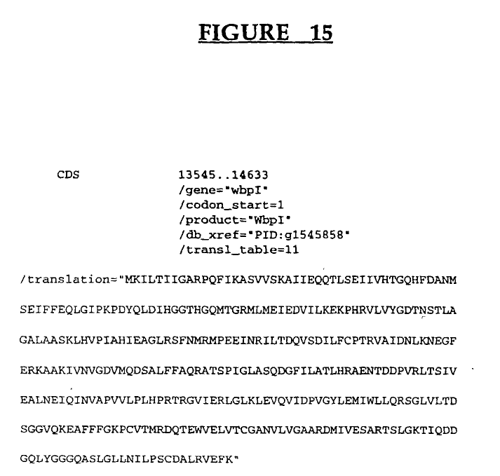

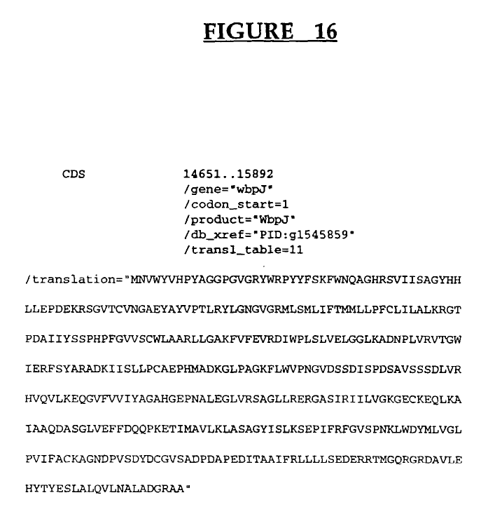

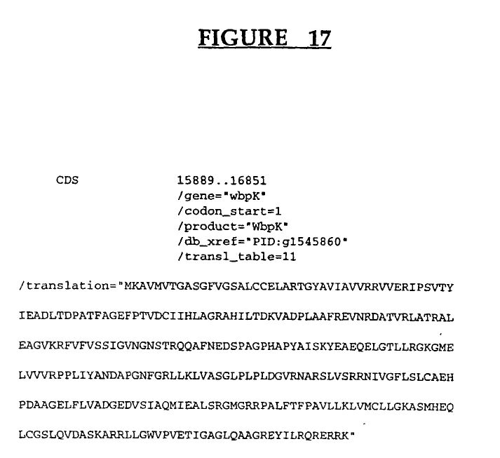

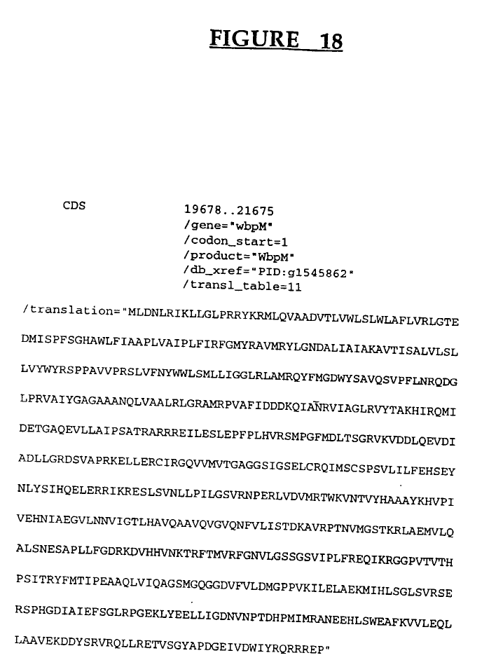

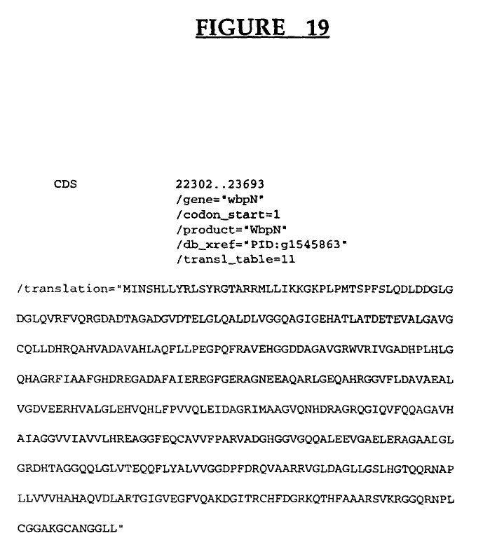

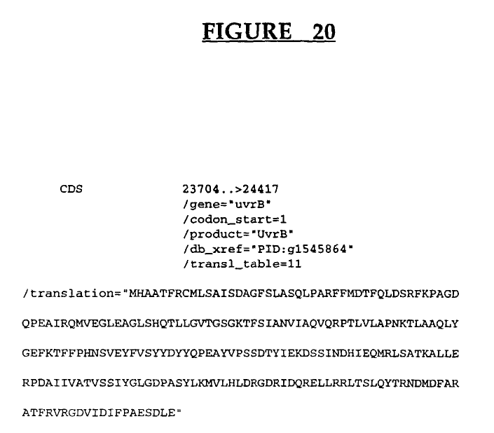

In an embodiment of the invention, an isolated nucleic acid molecule is provided having a sequence which encodes a protein having an amino acid sequence as shown in FIG. 3 or SEQ.ID. No.: 2; FIG. 4 or SEQ.ID. No.: 3; FIG. 5 or SEQ.ID. No.: 4; FIG. 6 or SEQ.ID. No.: 5; FIG. 7 or SEQ.ID. No.: 6; FIG. 8 or SEQ.ID. No.: 7; FIG. 9 or SEQ.ID. No.: 8; FIG. 10 or SEQ.ID. No.: 9; FIG. 11 or SEQ.ID. No.: 10; FIG. 12 or SEQ.ID. No.: 11; FIG. 13 or SEQ.ID. No.: 12; FIG. 14 or SEQ.ID. No.: 13; FIG. 15 or SEQ.ID. No.: 14; FIG. 16 or SEQ.ID. No.: 15; FIG. 17 or SEQ.ID. No.: 16.; FIG. 18 or SEQ.ID. No.: 17; FIG. 19 or SEQ.ID. No.: 18; and FIG. 20 or SEQ.ID. No.: 19.

Preferably, the purified and isolated nucleic acid molecule comprises

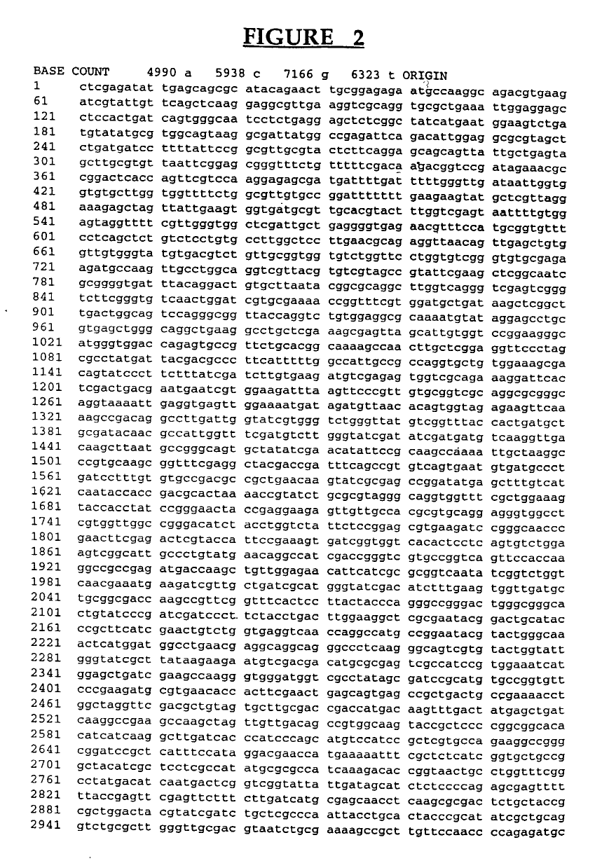

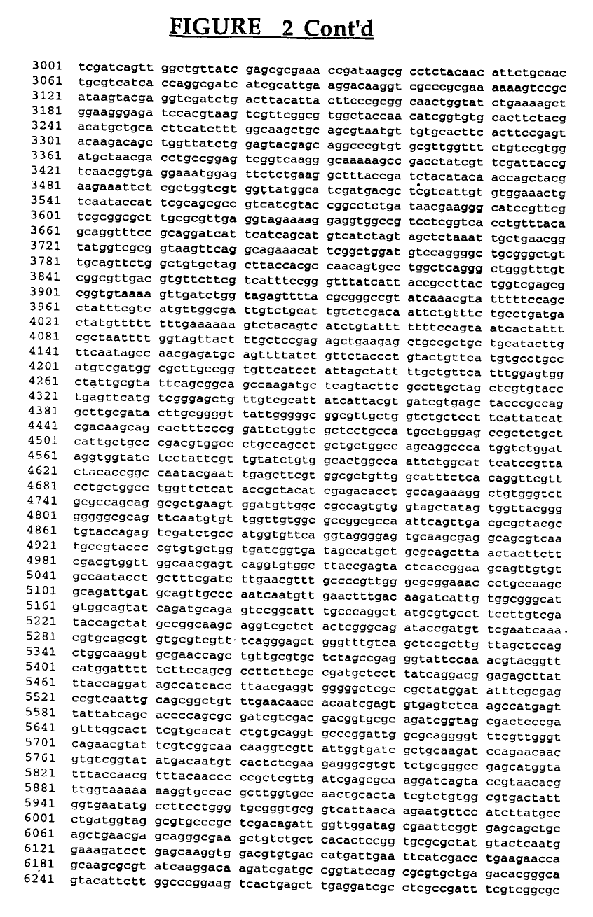

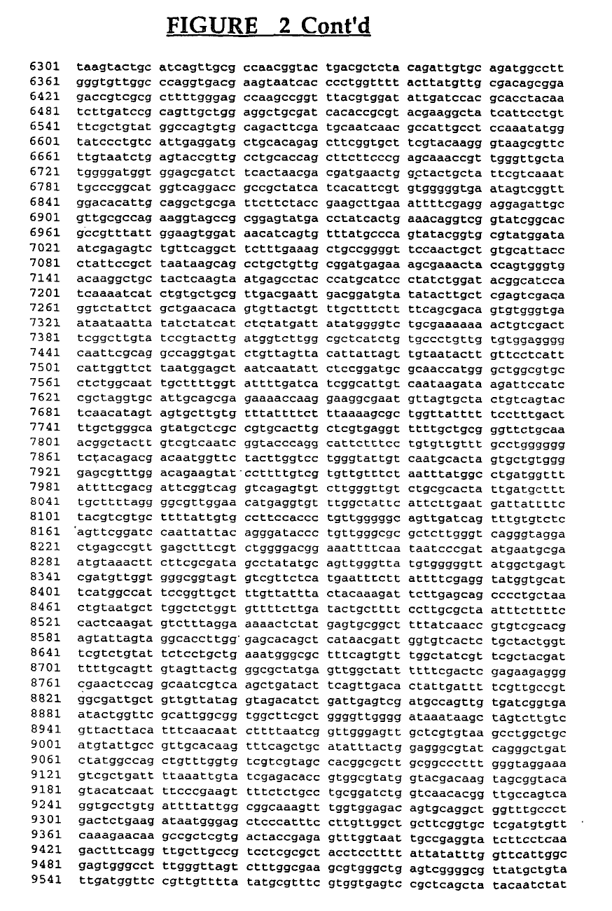

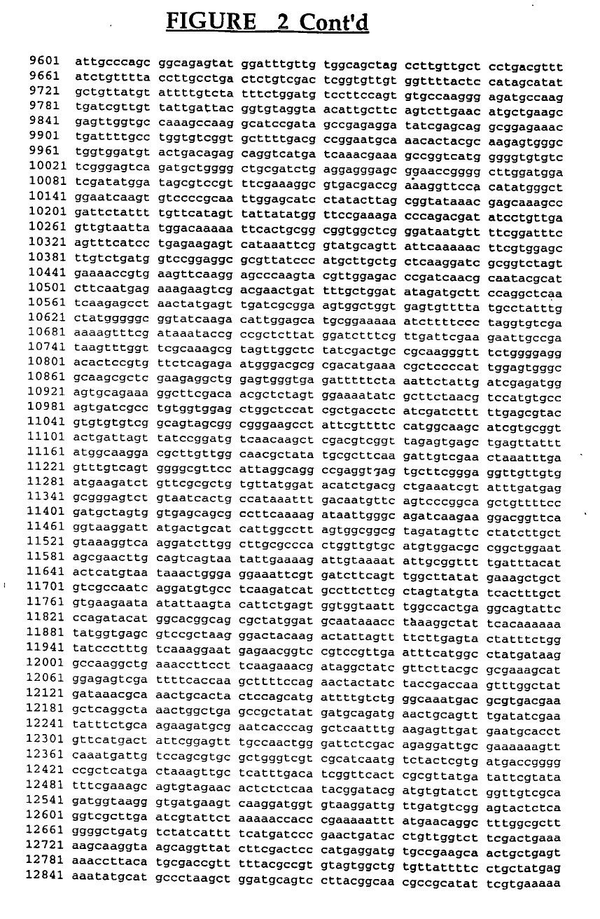

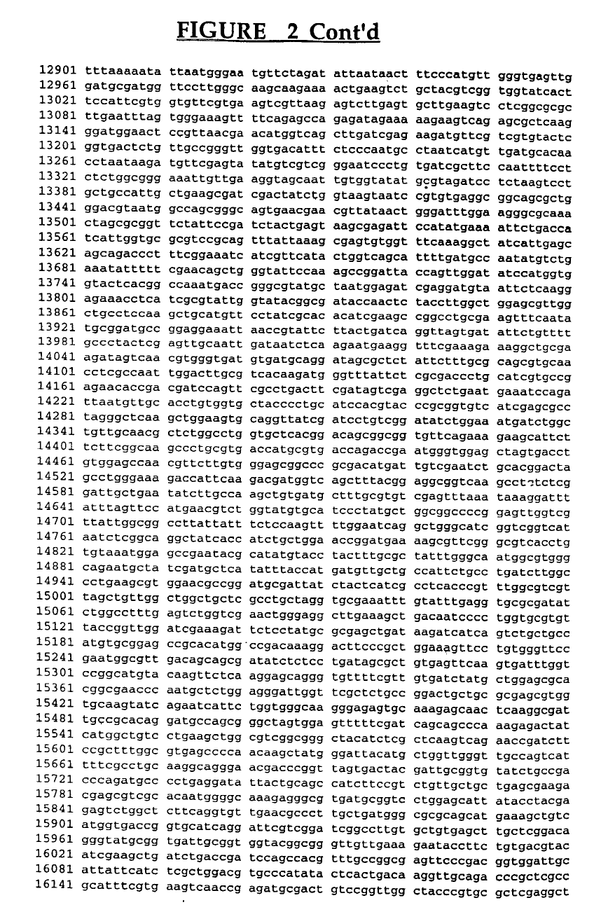

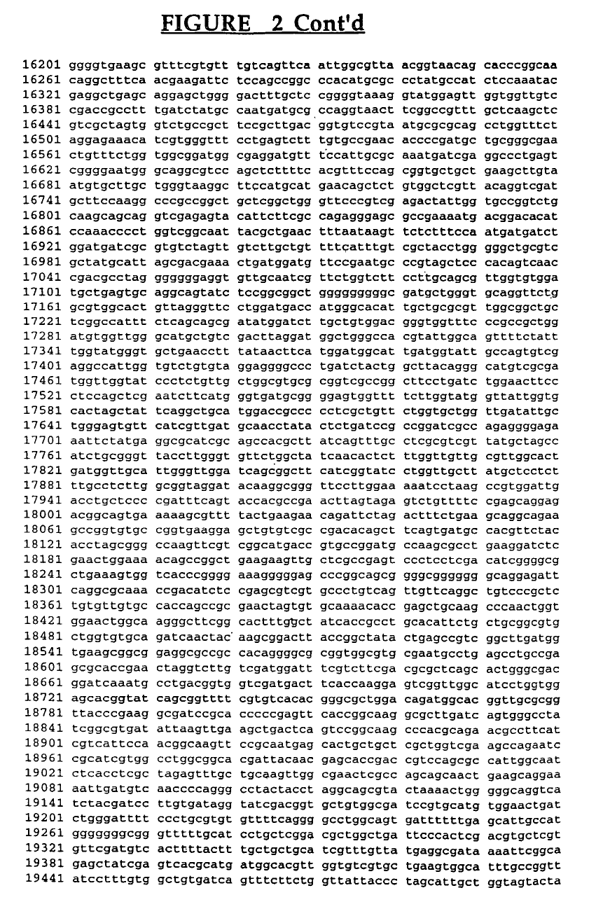

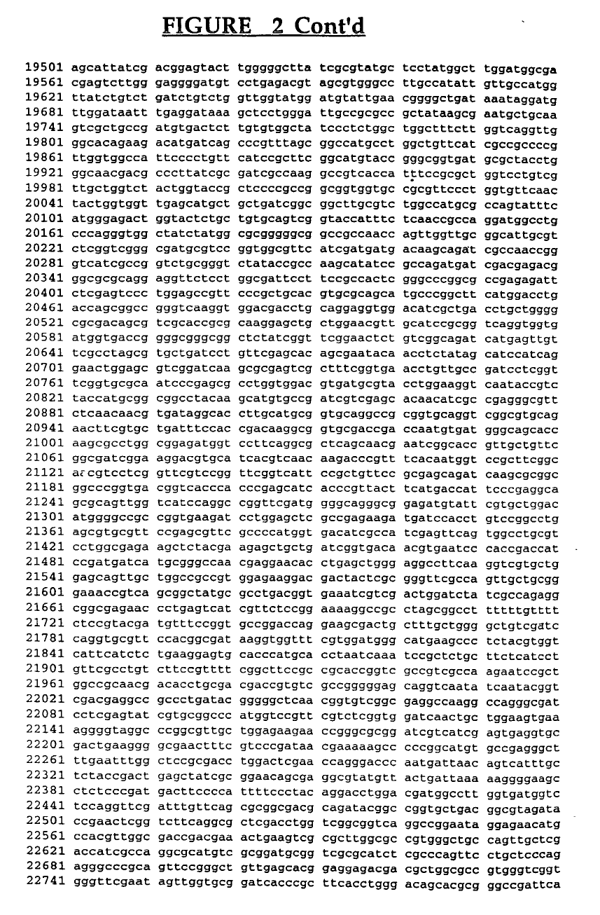

(a) a nucleic acid sequence containing nucleotides 1-479; 1286-2596; 2670-3620; 3689-5578; 5575-6066; 6152-6982; 7236-8552; 8549-9499; 9831-10388; 10388-11143; 11281-12411; 12427-13548; 13545-14633; 14651-15892; 15889-16851; 17935-19144; 19678-21675; 22302-23693; or 23704-24417, as shown in FIG. 2 or SEQ. ID. NO.: 1, wherein T can also be U;

(b) a nucleic acid sequence containing two or more of nucleotides 1-479; 1286-2596; 2670-3620; 3689-5578; 5575-6066; 6152-6982; 7236-8552; 8549-9499; 9830-10388; 10388-11143; 11281-12411; 12427-13548; 13545-14633; 14651-15892; 15889-16851; 17935-19144; 19678-21675; 22302-23693; or 23704-24417, as shown in FIG. 2 or SEQ. ID. NO.: 1, wherein T can also be U;

(c) nucleic acid sequences complementary to (a) or (b);

(d) nucleic acid sequences which are homologous to (a) or (b);

(e) a fragment of (a) to (d) that is at least 15 bases, preferably 20 to 30 bases, and which will hybridize to (a) to (d) under stringent hybridization conditions; or

(f) a nucleic acid molecule differing from any of the nucleic acids of (a) to (c) in codon sequences due to the degeneracy of the genetic code.

Specific embodiments of the nucleic acid molecule of the invention include the following:

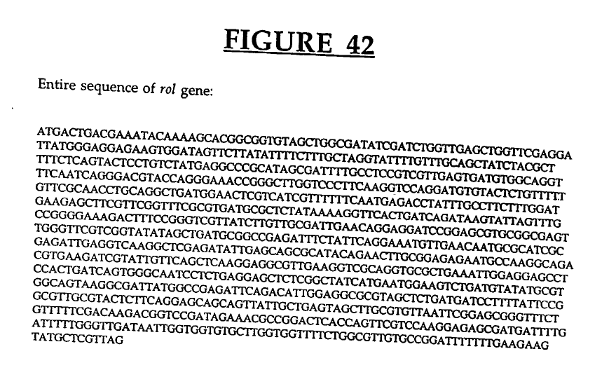

1. An isolated nucleic acid molecule characterized by having a sequence encoding a Rol (Wzz) protein of P. aeruginosa which regulates O-antigen linking. The nucleic acid molecule preferably encodes Rol having the amino acid sequence as shown in FIG. 3 or SEQ.ID. No.: 2, and most preferably comprises nucleotides 1-479 as shown in FIG. 2 or SEQ.ID. No.: 1, or a nucleotide sequence as shown in FIG. 42, which shows the full length nucleotide sequence of the rol gene.

2. An isolated nucleic acid molecule characterized by having a sequence encoding a PsbA (WbpA) protein of P. aeruginosa which has dehydrogenase activity. The nucleic acid molecule preferably encodes PsbA having the amino acid sequence as shown in FIG. 4 or SEQ.ID. No.: 3, and most preferably comprises nucleotides 1286-2596 as shown in FIG. 2 or SEQ.ID. No.: 1.

3. An isolated nucleic acid molecule characterized by having a sequence encoding a PsbB (WbpB) protein of P. aeruginosa. The nucleic acid molecule preferably encodes PsbB having the amino acid sequence as shown in FIG. 5 or SEQ.ID. No.: 4, and most preferably comprises nucleotides 2670-3620 as shown in FIG. 2 or SEQ.ID. No.: 1.

4. An isolated nucleic acid molecule characterized by having a sequence encoding a PsbC (WbpC) protein of P. aeruginosa which has acetyltransferase activity. The nucleic acid molecule preferably encodes PsbC having the amino acid sequence as shown in FIG. 6 or SEQ.ID. No.: 5, and most preferably comprises nucleotides 3689-5578 as shown in FIG. 2 or SEQ.ID. No.: 1.

5. An isolated nucleic acid molecule characterized by having a sequence encoding a PsbD (WbpD) protein of P. aeruginosa which has acetyltransferase activity. The nucleic acid molecule preferably encodes PsbD having the amino acid sequence as shown in FIG. 7 or SEQ.ID. No.: 6, and most preferably comprises nucleotides 5575-6066 as shown in FIG. 2 or SEQ.ID. No.: 1.

6. An isolated nucleic acid molecule characterized by having a sequence encoding a PsbE (WbpE) protein of P. aeruginosa. The nucleic acid molecule preferably encodes PsbE having the amino acid sequence as shown in FIG. 8 or SEQ.ID. No.: 7, and most preferably comprises nucleotides 6152-6982 as shown in FIG. 2 or SEQ.ID. No.: 1.

7. An isolated nucleic acid molecule characterized by having a sequence encoding a Rfc (Wzy) protein of P. aeruginosa which has O-polymerase activity. The nucleic acid molecule preferably encodes Rfc having the amino acid sequence as shown in FIG. 9 or SEQ.ID. No.: 8, and most preferably comprises nucleotides 7236-8552 as shown in FIG. 2 or SEQ.ID. No.: 1. The nucleic acid molecule may comprise nucleotides 7236 to 8552 where base 8059 is “G”. The Rfc coding region has a lower mol. % G+C than the P. aeruginosa chromosomal average and it has similar amino acid composition and codon usage to that reported for other Rfc proteins. Using a novel gene-replacement vector, the present inventors were able to generate PAO1 chromosomal rfc mutants. These knockout mutants express LPS containing complete core plus one O-repeat unit, indicating that they are no longer producing a functional O-polymerase enzyme.

8. An isolated nucleic acid molecule characterized by having a sequence encoding a PsbF (WbpF) protein of P. aeruginosa. The nucleic acid molecule preferably encodes PsbF having the amino acid sequence as shown in FIG. 10 or SEQ.ID. No.: 9, and most preferably comprises nucleotides 8549-9499 as shown in FIG. 2 or SEQ.ID. No.: 1.

9. An isolated nucleic acid molecule characterized by having a sequence encoding a PsbG (WbpG) protein of P. aeruginosa. The nucleic acid molecule preferably encodes PsbG having the amino acid sequence as shown in FIG. 13 or SEQ.ID. No.: 12, and most preferably comprises nucleotides 11281-12411 as shown in FIG. 2 or SEQ.ID. No.: 1.

The present inventors have inserted a gentamicin cassette into psbG which resulted in B-band deficient mutants of PAO1.

10. An isolated nucleic acid molecule characterized by having a sequence encoding a PsbH (WbpH) protein of P. aeruginosa which has ManA transferase activity. The nucleic acid molecule preferably encodes PsbH having the amino acid sequence as shown in FIG. 14 or SEQ.ID. No.: 13, and most preferably comprises nucleotides 12427-13548 as shown in FIG. 2 or SEQ.ID. No.: 1. The present inventors have produced a psbH knockout mutant of PAO1 which is B-band deficient.

11. An isolated nucleic acid molecule characterized by having a sequence encoding a PsbI (WbpI) protein of P. aeruginosa which converts UDP-N-acetylglucosamine to UDP-N-acetylmannosamine. The nucleic acid molecule preferably encodes PsbI having the amino acid sequence as shown in FIG. 15 or SEQ.ID. No.: 14, and most preferably comprises nucleotides 13545-14633 as shown in FIG. 2 or SEQ.ID. No.: 1.

12. An isolated nucleic acid molecule characterized by having a sequence encoding a PsbJ (WbpJ) protein of P. aeruginosa which has ManA transferase activity. The nucleic acid molecule preferably encodes PsbJ having the amino acid sequence as shown in FIG. 16 or SEQ.ID. No.: 15, and most preferably comprises nucleotides 14651-15892 as shown in FIG. 2 or SEQ.ID. No.: 1.

13. An isolated nucleic acid molecule characterized by having a sequence encoding a PsbK (WbpK) protein of P. aeruginosa which has dehydratase activity. The nucleic acid molecule preferably encodes PsbK having the amino acid sequence as shown in FIG. 17 or SEQ.ID. No.: 16, and most preferably comprises nucleotides 15889-16851 as shown in FIG. 2 or SEQ.ID. No.: 1.

14. An isolated nucleic acid molecule characterized by having a sequence encoding a PsbM (WbpM) protein of P. aeruginosa and having dehydrogenase activity. The nucleic acid molecule preferably encodes PsbM having the amino acid sequence as shown in FIG. 18 or SEQ.ID. No.: 17, and most preferably comprises nucleotides 19678-21675 as shown in FIG. 2 or SEQ.ID. No.: 1. PsbM knockout mutants do not produce LPS.

15. An isolated nucleic acid molecule characterized by having a sequence encoding a PsbN (WbpN) protein of P. aeruginosa. The nucleic acid molecule preferably encodes PsbN having the amino acid sequence as shown in FIG. 19 or SEQ.ID. No.: 18, and most preferably comprises nucleotides 22302-23693 as shown in FIG. 2 or SEQ.ID. No.: 1.

16. An isolated nucleic acid molecule characterized by having a sequence encoding a UvrB protein of P. aeruginosa which is involved in ultraviolet repair. The nucleic acid molecule preferably encodes UvrB having the amino acid sequence as shown in FIG. 20 or SEQ.ID. No.: 19, and most preferably comprises nucleotides 23704-24417 as shown in FIG. 2 or SEQ.ID. No.: 1.

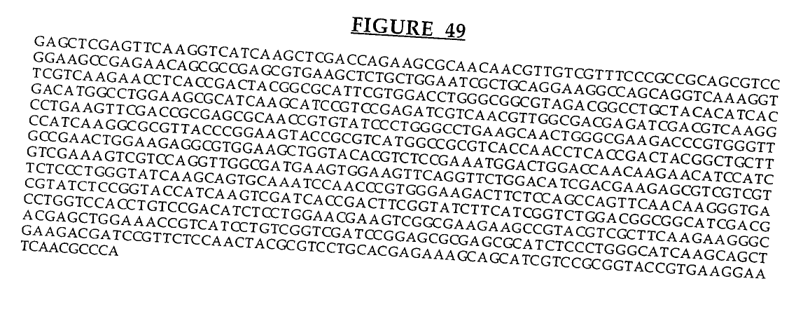

17. An isolated nucleic acid molecule characterized by having a sequence encoding a RpsA protein for a 30S ribosomal subunit. The nucleic acid molecule preferably encodes RpsA having the amino acid sequence as shown in FIG. 49.

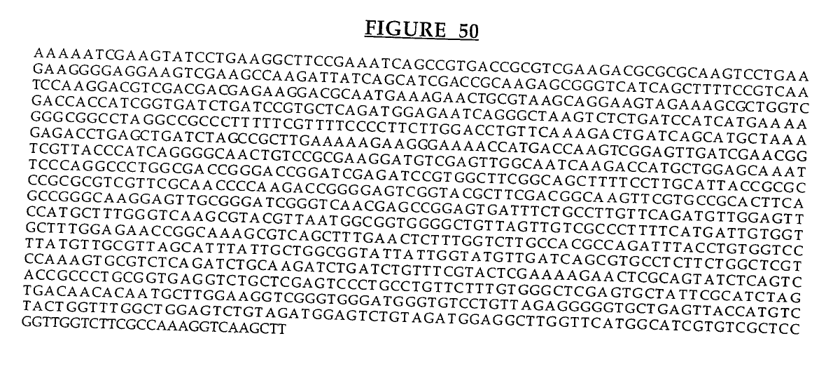

18. An isolated nucleic acid molecule characterized by having a sequence encoding a HimD protein for a host integration factor. The nucleic acid molecule preferably encodes HimD having the amino acid sequence as shown in FIG. 50.

In an embodiment of the invention, the nucleic acid molecule contains two genes from the gene cluster of the invention, preferably two genes which are adjacent in the gene cluster. For example, the present inventors have found that rfc (wzy) and psbF (wbpF) are cotranscribed and they are both required for B-band synthesis. If psbF (wbpF) is absent, both A and B synthesis are knocked out indicating that its gene product is required for expressor of A and B- band LPS onto the core oligosaccharide. Accordingly, the invention provides a nucleic acid molecule encoding a PsbF (WpbF) protein and an Rfc (Wzy) protein. Preferably a nucleic acid molecule comprising nucleotides 7239 to 9499 as shown in FIG. 2 or SEQ.ID. No.: 1.

It will be appreciated that the invention includes nucleic acid molecules encoding truncations of the proteins of the invention, and analogs and homologs of the proteins of the invention and truncations thereof, as described below. It will further be appreciated that variant forms of the nucleic acid molecules of the invention which arise by alternative splicing of an mRNA corresponding to a cDNA of the invention are encompassed by the invention.

Further, it will be appreciated that the invention includes nucleic acid molecules comprising nucleic acid sequences having substantial sequence homology with the nucleic acid sequences containing nucleotides 1-479; 1286-2596; 2670-3620; 3689-5578; 5575-6066; 6152-6982; 7236-8552; 8549-9499; 9831-10388; 10388-11143; 11281-12411; 12427-13548; 13545-14633; 14651-15892; 15889-16851; 17935-19144; 19678-21675; 22302-23693; or 23704-24417, as shown in FIG. 2 or SEQ. ID. NO.: 2 and fragments thereof. The term “sequences having substantial sequence homology” means those nucleic acid sequences which have slight or inconsequential sequence variations from these sequences, i.e. the sequences function in substantially the same manner to produce functionally equivalent proteins. The variations may be attributable to local mutations or structural modifications.

Nucleic acid sequences having substantial homology include nucleic acid sequences having at least 80-90%, preferably 90% identity with the nucleic acid sequence 1-479; 1286-2596; 2670-3620; 3689-5578; 5575-6066; 6152-6982; 7236-8552; 8549-9499; 9831-10388; 10388-11143; 11281-12411; 12427-13548; 13545-14633; 14651-15892; 15889-16851; 17935-19144; 19678-21675; 22302-23693; or 23704-24417, as shown in FIG. 2 or SEQ. ID. NO.: 2. By way of example, it is expected that a sequence having 80% sequence homology with the DNA sequence encoding PsbM of the invention will provide a functional PsbM protein.

Another aspect of the invention provides a nucleic acid molecule, and fragments thereof having at least 15 bases, which hybridizes to the nucleic acid molecules of the invention under hybridization conditions, preferably stringent hybridization conditions. Appropriate stringency conditions which promote DNA hybridization are known to those skilled in the art, or may be found in Current Protocols in Molecular Biology, John Wiley & Sons, N.Y. (1989), 6.3.1-6.3.6. For example, the following may be employed: 6.0×sodium chloride/sodium citrate (SSC) at about 45° C., followed by a wash of 2.0×SSC at 50° C. The stringency may be selected based on the conditions used in the wash step. For example, the salt concentration. in the wash step can be selected from a high stringency of about 0.2×SSC at 50° C. In addition, the temperature in the wash step can be at high stringency conditions, at about 65° C.

Isolated and purified nucleic acid molecules having sequences which differ from the nucleic acid sequence shown in SEQ ID NO:1 or FIG. 2, and the nucleic acid sequences 1-479; 1286-2596; 2670-3620; 3689-5578; 5575-6066; 6152-6982; 7236-8552; 8549-9499; 9831-10388; 10388-11143; 11281-12411; 12427-13548; 13545-14633; 14651-15892; 15889-16851; 17935-19144; 19678-21675; 22302-23693; or 23704-24417, as shown in FIG. 2 or SEQ. ID. NO.: 1, due to degeneracy in the genetic code are also within the scope of the invention. Such nucleic acids encode functionally equivalent proteins (e.g., a PsbM (WpbM) protein having dehydrogenase activity) but differ in sequence from the above mentioned sequences due to degeneracy in the genetic code.

An isolated nucleic acid molecule of the invention which comprises DNA can be isolated by preparing a labelled nucleic acid probe based on all or part of the nucleic acid sequences containing nucleotides 1-479; 1286-2596; 2670-3620; 3689-5578; 5575-6066; 6152-6982; 7236-8552; 8549-9499; 9831-10388; 10388-11143; 11281-12411; 12427-13548; 13545-14633; 14651-15892; 15889-16851; 17935-19144; 19678-21675; 22302-23693; or 23704-24417, as shown in FIG. 2 or SEQ. ID. NO.: 2, and using this labelled nucleic acid probe to screen an appropriate DNA library (e.g. a cDNA or genomic DNA library). For example, a whole genomic library isolated from a microorganism, such as a serotype of P. aeruginosa, can be used to isolate a DNA encoding a novel protein of the invention by screening the library with the labelled probe using standard techniques. Nucleic acids isolated by screening of a cDNA or genomic DNA library can be sequenced by standard techniques.

An isolated nucleic acid molecule of the invention which is DNA can also be isolated by selectively amplifying a nucleic acid encoding a novel protein of the invention using the polymerase chain reaction (PCR) methods and cDNA or genomic DNA. It is possible to design synthetic oligonucleotide primers from the nucleic acid molecules containing the nucleotides 1-479; 1286-2596; 2670-3620; 3689-5578; 5575-6066; 6152-6982; 7236-8552; 8549-9499; 9831-10388; 10388-11143; 11281-12411; 12427-13548; 13545-14633; 14651-15892; 15889-16851; 17935-19144; 19678-21675; 22302-23693; or 23704-24417, as shown in FIG. 2 or SEQ. ID. NO.: 2, for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. It will be appreciated that cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a novel protein of the invention into an appropriate vector which allows for transcription of the cDNA to produce an RNA molecule which encodes a novel protein of the invention. For example, a cDNA can be cloned downstream of a bacteriophage promoter, (e.g. a T7 promoter) in a vector, cDNA can be transcribed in vitro with T7 polymerase, and the resultant RNA can be isolated by standard techniques.

A nucleic acid molecule of the invention may also be chemically synthesized using standard techniques. Various methods of chemically synthesizing polydeoxynucleotides are known, including solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule encodes a novel protein of the invention may be accomplished by expressing the cDNA in an appropriate host cell by standard techniques, and testing the activity of the protein using the methods as described herein. For example, the activity of a putative PsbM protein may be tested by mixing with an appropriate substrate and assaying for dehydrogenase activity. A cDNA having the activity of a novel protein of the invention so isolated can be sequenced by standard techniques, such as dideoxynucleotide chain termination or Maxam-Gilbert chemical sequencing, to determine the nucleic acid sequence and the predicted amino acid sequence of the encoded protein.

The initiation codon and untranslated sequences of the nucleic acid molecules of the invention may be determined using currently available computer software designed for the purpose, such as PC/Gene (IntelliGenetics Inc., Calif.). Regulatory elements can be identified using conventional techniques. The function of the elements can be confirmed by using these elements to express a reporter gene which is operatively linked to the elements. These constructs may be introduced into cultured cells using standard procedures. In addition to identifying regulatory elements in DNA, such constructs may also be used to identify proteins interacting with the elements, using techniques known in the art.

The sequence of a nucleic acid molecule of the invention may be inverted relative to its normal presentation for transcription to produce an antisense nucleic acid molecule. Preferably, an antisense sequence is constructed by inverting a region preceding the initiation codon or an unconserved region. In particular, the nucleic acid sequences contained in the nucleic acid molecules of the invention or a fragment thereof, preferably one or more of the nucleic acid sequences shown in the Sequence Listing as SEQ. ID. NO. 1 and in FIG. 2 (i.e. a nucleic acid molecule containing nucleotides 1-479; 1286-2596; 2670-3620; 3689-5578; 5575-6066; 6152-6982; 7236-8552; 8549-9499; 9831-10388; 10388-11143; 11281-12411; 12427-13548; 13545-14633; 14651-15892; 15889-16851; 17935-19144; 19678-21675; 22302-23693; or 23704-24417) may be inverted relative to their normal presentation for transcription to produce antisense nucleic acid molecules.

The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The invention also provides nucleic acids encoding fusion proteins comprising a novel protein of the invention and a selected protein, or a selectable marker protein (see below).

II. Novel Proteins of the Invention

The invention further broadly contemplates an isolated protein characterized in that it has part or all of the primary structural conformation (ie. continuous sequence of amino acid residues) of a novel protein encoded by a gene of the psb gene cluster of the invention. In an embodiment of the invention, an isolated protein is provided which has the amino acid sequence as shown in FIG. 3 or SEQ ID NO:2; (Rol or Wzz), FIG. 4 or SEQ ID NO:3 (PsbA or WbpA) FIG. 5 or SEQ ID NO:4 (PsbB or WbpB); FIG. 6 or SEQ ID NO:5 (PsbC or WbpC); FIG. 7 or SEQ ID N0:6 (PsbD or WbpD); FIG. 8 or SEQ ID NO:7 (PsbE or WbpE); FIG. 9 or SEQ ID NO:8 (Rfc or Wzy); FIG. 10 or SEQ ID NO:9 (PsbF or WbpF); FIG. 11 or SEQ ID NO:10 (HisH); FIG. 12 or SEQ ID NO:11 (HisF); FIG. 13 or SEQ ID NO:12 (PsbG or WbpG); FIG. 14 or SEQ ID NO:13 (PsbH or WbpH); FIG. 15 or SEQ ID NO:14 (PsbI or WbpI); FIG. 16 or SEQ ID NO:15 (PsbJ or WbpJ); FIG. 17 or SEQ ID NO:16 (PsbK or WbpK); FIG. 18 or SEQ ID NO:17 (PsbM or WbpM); FIG. 19 or SEQ ID NO:18 (PsbN or WbpN); or FIG. 20 or SEQ ID NO:19 (UvrB).

The gene products of rol, psbA, psbB, psbC, psbD, psbE, rfc, psbF, hisH, hisF, psbG, psbH, psbi, psbJ, psbL, and psbK (also known as wzz, wbpA, wbpB, wbpC, wbpD, wbpE, wzy, wbpF, hisH, hisF, wbpG, wbpH, wpbi, wbpj respectively) are expected to be found in serotypes O2, O5, O16, O18, and O20, and the gene products of psbM and psbN (also known as wbpM and wbpN, respectively) are expected to be found in serotypes O1 to O20. The gene products of hisF and hisH are not found in serotype O6.

Specific embodiments of the invention include the following:

1. An isolated Rol (Wzz) protein of P. aeruginosa which regulates O-antigen linking, having the amino acid sequence as shown in FIG. 3 or SEQ.ID. No.: 2. The function of Rol may be associated with the Rfc protein.

2. An isolated PsbA (WbpA) protein of P. aeruginosa which has dehydrogenase activity, and the amino acid sequence as shown in FIG. 4 or SEQ.ID. No.: 3. PsbA may be involved in the biosynthesis of mannuronic acid residues.

3. An isolated PsbB (WbpB) protein of P. aeruginosa having the amino acid sequence as shown in FIG. 5 or SEQ.ID. No.: 4. PsbB may be involved in Fuc2NAc biosynthesis.

4. An isolated PsbC (WbpC) protein of P. aeruginosa which has acetyltransferase activity and the amino acid sequence as shown in FIG. 6 or SEQ.ID. No.: 5. PsbC may be involved in the acetylation of mannuronic acid residues in the O-antigen.

5. An isolated PsbD (WbpD) protein of P. aeruginosa which has acetyltransferase activity and the amino acid sequence as shown in FIG. 7 or SEQ.ID. No.: 6. PsbD may be involved in the acetylation of mannuronic acid residues in the O-antigen.

6. An isolated PsbE (WbpE) protein of P. aeruginosa. having the amino acid sequence as shown in FIG. 8 or SEQ.ID. No.: 7. PsbE may be involved in the biosynthesis of 2,3-, 2,4-, and 2,6-dideoxy sugars such as 2,3-dideoxy mannuronic acid produced by P. aeruginosa O5.

7. An isolated Rfc (Wzy) protein of P. aeruginosa which has O-polymerase activity and the amino acid sequence as shown in FIG. 9 or SEQ.ID. No.: 8. The Rfc protein is characterized as very hydrophobic, and it is an integral membrane protein with 11 putative membrane spanning domains.

8. An isolated PsbF (WbpF) protein of P. aeruginosa. having the amino acid sequence as shown in FIG. 10 or SEQ.ID. No.: 9. PsbF is translationally coupled with rfc and it is a putative flippase.

9. An isolated PsbG (WbpG) protein of P. aeruginosa which has the amino acid sequence as shown in FIG. 13 or SEQ.ID. No.: 12.

10. An isolated PsbH (WbpH) protein of P. aeruginosa which has ManA transferase activity and the amino acid sequence as shown in FIG. 14 or SEQ.ID. No.: 13. PsbH may be involved in the addition of ManA (i.e. Man(2NAc3N)A) to the O-antigen unit.

11. An isolated PsbI (WbpI) protein of P. aeruginosa which converts UDP-N-acetylglucosamine to UDP-N-acetylmannosamine, and has the amino acid sequence as shown in FIG. 15 or SEQ.ID. No.: 14.

12. An isolated PsbJ (WbpJ) protein of P. aeruginosa which has ManA transferase activity, and the amino acid sequence as shown in FIG. 16 or SEQ.ID. No.: 15. Based on their gene order and their relative hydropathic indices, the psbj and psbH gene products are thought to transfer Man(NAc)2A and Man(2Nac3N)A, respectively.

13. An isolated PsbK (WbpK) protein of P. aeruginosa which has dehydratase activity, and the amino acid sequence as shown in FIG. 17 or SEQ.ID. No.: 16.

14. An isolated PsbM (WbpM) protein of P. aeruginosa having dehydrogenase activity, and the amino acid sequence as shown in FIG. 18 or SEQ.ID. No.: 17. PsbM is involved in the biosynthesis of N-acetylfucosamine residues of the O-antigen. PsbM contains 2 NAD binding domains.

15. An isolated PsbN (WbpN) protein of P. aeruginosa. having the amino acid sequence as shown in FIG. 19 or SEQ.ID. No.: 18.

16. An UvrB protein of P. aeruginosa which is involved in ultraviolet repair and has the amino acid sequence as shown in FIG. 20 or SEQ.ID. No.: 19.

The molecular weights, isoelectric points, and hydropathic indices of the Rol (Wzz), PsbA (WbpA), PsbB (WbpB), PsbC (WbpC), PsbD (WbpD), PsbE (WbpE), Rfc (Wzy), PsbF (WbpF), PsbG (WbpG), PsbH (WbpH), PsbI (WbpI), PsbJ (WbpJ), PsbK (WbpK), PsbM (WbpM) and PsbN (WbpN) proteins are shown in Table 1.

Within the context of the present invention, a protein of the invention may include various structural forms of the primary protein which retain biological activity. For example, a protein of the invention may be in the form of acidic or basic salts or in neutral form. In addition, individual amino acid residues may be modified by oxidation or reduction.

In addition to the full length amino acid sequences (FIGS. 3 to 20 or SEQ. ID.NOS:2 to 19), the proteins of the present invention may also include truncations of the proteins, and analogs, and homologs of the proteins and truncations thereof as described herein. Truncated proteins may comprise peptides of at least fifteen amino acid residues.

The proteins of the invention may also include analogs of the proteins having the amino acid sequences shown in FIGS. 3 to 20, or SEQ.ID. NOS: 2 to 19 and/or truncations thereof as described herein, which may include, but are not limited to an amino acid sequence containing one or more amino acid substitutions, insertions, and/or deletions. Amino acid substitutions may be of a conserved or non-conserved nature. Conserved amino acid substitutions involve replacing one or more amino acids of the proteins of the invention with amino acids of similar charge, size, and/or hydrophobicity characterisitics. When only conserved substitutions are made the resulting analog should be functionally equivalent. Non-conserved substitutions involve replacing one or more amino acids of the amino acid sequence with one or more amino acids which possess dissimilar charge, size, and/or hydrophobicity characteristics.

One or more amino acid insertions may be introduced into the amino acid sequences shown in FIGS. 3 to 20, or SEQ.ID. NOS:2 to 19. Amino acid insertions may consist of single amino acid residues or sequential amino acids ranging from 2 to 15 amino acids in length. For example, amino acid insertions may be used to destroy target sequences so that the protein is no longer active. This procedure may be used in vivo to inhibit the activity of a protein of the invention.

Deletions may consist of the removal of one or more amino acids, or discrete portions from the amino acid sequences shown in FIGS. 3 to 20 or SEQ.ID. NOS:2 to 19. The deleted amino acids may or may not be contiguous. The lower limit length of the resulting analog with a deletion mutation is about 10 amino acids, preferably 100 amino acids.

Analogs of a protein of the invention may be prepared by introducing mutations in the nucleotide sequence encoding the protein. Mutations in nucleotide sequences constructed for expression of analogs of a protein of the invention must preserve the reading frame of the coding sequences. Furthermore, the mutations will preferably not create complementary regions that could hybridize to produce secondary mRNA structures, such as loops or hairpins, which could adversely affect translation of the receptor mRNA.

Mutations may be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site specific mutagenesis procedures may be employed to provide an altered gene having particular codons altered according to the substitution, deletion, or insertion required. Deletion or truncation of a protein of the invention may also be constructed by utilizing convenient restriction endonuclease sites adjacent to the desired deletion. Subsequent to restriction, overhangs may be filled in, and the DNA religated. Exemplary methods of making the alterations set forth above are disclosed by Sambrook et al (Molecular Cloning: A Laboratory Manual, 2nd Ed., Cold Spring Harbor Laboratory Press, 1989).

The proteins of the invention also include homologs of the amino acid sequences shown in FIGS. 3 to 20, or SEQ.ID. NOS:2 to 19 and/or truncations thereof as described herein. Such homologs are proteins whose amino acid sequences are comprised of amino acid sequences that hybridize under stringent hybridization conditions (see discussion of stringent hybridization conditions herein) with a probe used to obtain a protein of the invention. Homologs of a protein of the invention will have the same regions which are characteristic of the protein.

Amino acid homologies for WbpA, WbpD, WbpE, HisH, HisF, WbpI, WbpJ, WbpK, WbpM and Wzz proteins are shown in Table 2 to 4. It will be appreciated that the invention includes WbpA, WbpD, WbpE, HisH, HisF, WbpI, Wbpj, WbpK, WbpM and Wzz proteins having at least 51%, 84%, 76%, 57%, 54%, 70%, 53%, 54%, 61% and 51% homology, respectively.

The invention also contemplates isoforms of the proteins of the invention. An isoform contains the same number and kinds of amino acids as a protein of the invention, but the isoform has a different molecular structure. The isoforms contemplated by the present invention are those having the same properties as a protein of the invention as described herein.

The present invention also includes a protein of the invention conjugated with a selected protein, or a selectable marker protein (see below) to produce fusion proteins. Additionally, immunogenic portions of a protein of the invention are within the scope of the invention.

The proteins of the invention (including truncations, analogs, etc.) may be prepared using recombinant DNA methods. Accordingly, the nucleic acid molecules of the present invention having a sequence which encodes a protein of the invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, means that the expression vectors contain a nucleic acid molecule of the invention and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The invention therefore contemplates a recombinant expression vector of the invention containing a nucleic acid molecule of the invention, or a fragment thereof, and the necessary regulatory sequences for the transcription and translation of the inserted protein-sequence. Suitable regulatory sequences may be derived from a variety of sources, including bacterial, fungal, or viral genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein and/or its flanking regions.

The invention further provides a recombinant expression vector comprising a DNA nucleic acid molecule of the invention cloned into the expression vector in an antisense orientation. That is, the DNA molecule is operatively linked to a regulatory sequence in a manner which allows for expression, by transcription of the DNA molecule, of an RNA molecule which is antisense to a nucleotide sequence comprising 1-479; 1293-2596; 2670-3620; 3277-5577; 5574-6065; 6151-6981; 7235-8551; 8548-9498; 9830-10388; 10388-11143; 11281-12411; 12427-13548; 13545-14633; 14651-15892; 15889-16851; 18032-19141; 19678-21675; 22302-23693; or 23704-24417, as shown in FIG. 2 or SEQ. ID. NO.: 2. Regulatory sequences operatively linked to the antisense nucleic acid can be chosen which direct the continuous expression of the antisense RNA molecule.

The recombinant expression vectors of the invention may also contain a selectable marker gene which facilitates the selection of host cells transformed or transfected with a recombinant molecule of the invention. Examples of selectable marker genes are genes encoding a protein such as,G418 and hygromycin which confer resistance to certain drugs, β-galactosidase, chloramphenicol acetyltransferase, or firefly luciferase. Transcription of the selectable marker gene is monitored by changes in the concentration of the selectable marker protein such as O-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker g( ne will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of a target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-tranferase (GST), maltose E binding protein, or protein A, respectively, to the recombinant protein.

Recombinant expression vectors can be introduced into host cells to produce a transformant host cell. The term “transformant host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for examples electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells or mammalian cells. Other suitable host cells can be found in Goeddel, Gene Expression Technology: Methods in Enzymology 185, Academic Press, San Diego, Calif. (199 1).

More particularly, bacterial host cells suitable for carrying out the present invention include E. coli, as well as many other bacterial species well known to one of ordinary skill in the art. Bacterial expression vectors preferably comprise a promoter which functions in the host cell, one or more selectable phenotypic markers, and a bacterial origin of replication. Representative promoters include the μ-lactamase (penicillinase) and lactose promoter system (see Chang et al., Nature 275:615, 1978), the trp promoter (Nichols and Yanofsky, Meth in Enzymology 101:155, 1983) and the tac promoter (Russell et al., Gene 20: 231, 1982). Representative selectable markers include various antibiotic resistance markers such as the kanamycin or ampicillin resistance genes. Suitable expression vectors include but are not limited to bacteriophages such as lambda derivatives or plasmids such as pBR322 (see Bolivar et al., Gene 2:9S, 1977), the pUC plasmids pUC18, pUC19, pUC118, pUC119 (see Messing, Meth in Enzymology 101:20-77, 1983 and Vieira and Messing, Gene 19:259-268, 1982), and pNH8A, pNH16a, pNH18a, and Bluescript M13 (Stratagene, La Jolla, Calif.).

Yeast and fungi host cells suitable for carrying out the present invention include, but are not limited to Saccharomyces cerevisae, the genera Pichia or Kluyveromyces and various species of the genus Aspergillus. Examples of vectors for expression in yeast S. cerivisae include pYepSecl (Baldari. et al., (1987) Embo J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Protocols for the transformation of yeast and fungi are well known to those of ordinary skill in the art.(see Hinnen et al., PNAS USA 75:1929, 1978; Itoh et al., J. Bacteriology 153:163, 1983, and Cullen et al. (Bio/Technology 5:369, 1987).

The proteins of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

III. Applications

Detection of Nucleic Acid Molecules, Antibodies, and Diagnostic Applications

The nucleic acid molecules of the invention, allow those skilled in the art to construct nucleotide probes for use in the detection of nucleotide sequences in a sample. A nucleotide probe may be labelled with a detectable marker such as a radioactive label which provides for an adequate signal and has sufficient half life such as ³²P, ³H, ¹⁴C or the like. Other detectable markers which may be used, include antigens that are recognized by a specific labelled antibody, fluorescent compounds, enzymes, antibodies specific for a labelled antigen, and chemiluminescent compounds. An appropriate label may be selected having regard to the rate of hybridization and binding of the probe to the nucleotide to be detected and the amount of nucleotide available for hybridization.

The nucleotide probes may be used to detect genes that encode proteins related to or analogous to proteins of the invention.

Accordingly, the present invention also relates to a method of detecting the presence of nucleic acid molecules encoding a protein of the invention in a sample comprising contacting the sample under hybridization conditions with one or more of nucleotide probes which hybridize to the nucleic acid molecules and are labelled with a detectable marker, and determining the degree of hybridization between the nucleic acid molecule in the sample and the nucleotide probes.

In an embodiment of the invention a method for detecting P. aeruginosa serotypes O1 to O20 in a sample comprising contacting the sample with a nucleotide sequence encoding PsbM, or PsbN, or a fragment thereof, under conditions which permit the nucleic acid molecule to hybridize with a complementary sequence in the sample to form a hybridization product, and assaying for the hybridization product.

In another embodiment of the invention a method for detecting P.aeruginosa serotypes O2, O5, O16, O18, O20 in a sample comprising contacting the sample with a nucleotide sequence encoding one or more of Rol, PsbB, PsbC, PsbD, PsbE, rfc, PsbF, PsbG, PsbH, PsbI, PsbJ, PsbK (also known as Wzz, WbpB, WbpC, WbpD, WbpE, Wzy, WbpF, WbpG, WbpH, WbpI, WbpJ, WbpK, respectively), HisH, or HisF or a fragment thereof, under conditions which permit the nucleic acid molecule to hybridize with complementary sequences in the sample to form hybridization products, and assaying for the hybridization products.

Hybridization conditions which may be used in the methods of the invention are known in the art and are described for example in Sambrook J, Fritch E F, Maniatis T. In: Molecular Cloning, A Laboratory Manual,1989. (Nolan C, Ed.), Cold Spring Harbor Laboratory Press, Cold Spring Harbor, N.Y. The hybridization product may be assayed using techniques known in the art. The nucleotide probe may be labelled with a detectable marker as described herein and the hybridization product may be assayed by detecting the detectable marker or the detectable change produced by the detectable marker.

The nucleic acid molecule of the invention also permits the identification and isolation, or synthesis of nucleotide sequences which may be used as primers to amplify a nucleic acid molecule of the invention, for example in the polymerase chain reaction (PCR) which is discussed in more detail below. The primers may be used to amplify the genomic DNA of other bacterial species known to have LPS. The PCR amplified sequences can be examined to determine the relationship between the various LPS genes.

The length and bases of the primers for use in the PCR are selected so that they will hybridize to different strands of the desired sequence and at relative positions along the sequence such that an extension product synthesized from one primer when it is separated from its template can serve as a template for extension of the other primer into a nucleic acid of defined length.

Primers which may be used in the invention are oligonucleotides i.e. molecules containing two or more deoxyribonucleotides of the nucleic acid molecule of the invention which occur naturally as in a purified restriction endonuclease digest or are produced synthetically using techniques known in the art such as for example phosphotriester and phosphodiester methods (See Good et al Nucl. Acid Res 4:2157, 1977) or automated techniques (See for example, Conolly, B A. Nucleic Acids Res. 15:15(7): 3131, 1987). The primers are capable of acting as a point of initiation of synthesis when placed under conditions which permit the synthesis of a primer extension product which is complementary to the DNA sequence of the invention i.e. in the presence of nucleotide substrates, an agent for polymerization such as DNA polymerase and at suitable temperature and pH. Preferably, the primers are sequences that do not form secondary structures by base pairing with other copies of the primer or sequences that form a hair pin configuration. The primer preferably contains between about 7 and 25 nucleotides.

The primers may be labelled with detectable markers which allow for detection of the amplified products. Suitable detectable markers are radioactive markers such as P-32, S-35, 1-125, and H-3, luminescent markers such as chemiluminescent markers, preferably luminol, and fluorescent markers, preferably dansyl chloride, fluorcein-5-isothiocyanate, and 4-fluor-7-nitrobenz-2-axa-1,3 diazole, enzyme markers such as horseradish peroxidase, alkaline phosphatase, O-galactosidase, acetylcholinesterase, or biotin.

It will be appreciated that the primers may contain non-complementary sequences provided that a sufficient amount of the primer contains a sequence which is complementary to a nucleic acid molecule of the invention or oligonucleotide fragment thereof, which is to be amplified. Restriction site linkers may also be incorporated into the primers allowing for digestion of the amplified products with the appropriate restriction enzymes facilitating cloning and sequencing of the amplified product.

In an embodiment of the invention a method of determining the presence of a nucleic acid molecule having a sequence encoding a protein of the invention is provided comprising treating the sample with primers which are capable of amplifying the nucleic acid molecule or a predetermined oligonucleotide fragment thereof in a polymerase chain reaction to form amplified sequences, under conditions which permit the formation of amplified sequences and, assaying for amplified sequences.

In a preferred embodiment of the invention, a method for detecting P. aeruginosa serotypes O1 to O20 in a sample is provided comprising treating the sample with a primer which is capable of amplifying nucleic acid molecules comprising nucleotide sequences encoding PsbM (WbpM), or PsbN (WbpN), or a predetermined oligonucleotide fragment thereof, in a polymerase chain reaction to form amplified sequences, under conditions which permit the formation of amplified sequences and, assaying for amplified sequences.

In another preferred embodiment of the invention, a method for detecting P. aeruginosa serotypes O2, O5, O16, O18, O20 in a sample is provided comprising treating the sample with a primer which is capable of amplifying nucleic acid molecules comprising nucleotide sequences encoding Rol, PsbA, PsbB, PsbC, PsbD, PsbE, Rfc, PsbF, PsbG, PsbH, PsbI, PsbJ, PsbK, (also known as Wzz, WbpA, WbpB, WbpC, WbpD, WbpE, Wzy, WbpF, WbpG, WbpH, WbpI, WbpJ, WbpK respectively) HisH or HisF, or a predetermined oligonucleotide fragment thereof, in a polymerase chain reaction to form amplified sequences, under conditions which permit the formation of amplified sequences and, assaying for amplified sequences.

The polymerase chain reaction refers to a process for amplifying a target nucleic acid sequence as generally described in Innis et al, Academic Press, 1990 in Mullis el al., U.S. Pat. No. 4,863,195 and Mullis, U.S. Pat. No. 4,683,202 which are incorporated herein by reference. Conditions for amplifying a nucleic acid template are described in M. A. Innis and D. H. Gelfand, PCR Protocols, A Guide to Methods and Applications M. A. Innis, D. H. Gelfand, J. J. Sninsky and T. J. White eds, pp3-12, Academic Press 1989, which is also incorporated herein by reference.

The amplified products can be isolated and distinguished based on their respective sizes using techniques known in the art. For example, after amplification, the DNA sample can be separated on an agarose gel and visualized, after staining with ethidium bromide, under ultra violet (UW) light. DNA may be amplified to a desired level and a further extension reaction may be performed to incorporate nucleotide derivatives having detectable markers such as radioactive labelled or biotin labelled nucleoside triphosphates. The primers may also be labelled with detectable markers as discussed above. The detectable markers may be analyzed by restriction and electrophoretic separation or other techniques known in the art.

The conditions which may be employed in the methods of the invention using PCR are those which permit hybridization and amplification reactions to proceed in the presence of DNA in a sample and appropriate complementary hybridization primers. Conditions suitable for the polymerase chain reaction are generally known in the art. For example, see M. A. Innis and D. H. Gelfand, PCR Protocols, A guide to Methods and Applications M. A. Innis, D. H. Gelfand, J. J. Sninsky and T. J. White eds, pp3-12, Academic Press 1989, which is incorporated herein by reference. Preferably, the PCR utilizes polymerase obtained from the thermophilic bacterium Thermus aquatics (Taq polymerase, GeneAmp Kit, Perkin Elmer Cetus) or other thermostable polymerase may be used to amplify DNA template strands.

It will be appreciated that other techniques such as the Ligase Chain Reaction (LCR) and NASBA may be used to amplify a nucleic acid molecule of the invention (Barney in “PCR Methods and Applications”, August 1991, Vol.1(1), page 5, and European Published Application No. 0320308, published Jun. 14, 1989, and U.S. Ser. Pat. No. 5,130,238 to Malek).

A protein of the invention can be used to prepare antibodies specific for the protein. Antibodies can be prepared which bind a distinct epitope in an unconserved region of the protein. An unconserved region of the protein is one which does not have substantial sequence homology to other proteins. Alternatively, a region from a well-characterized domain can be used to prepare an antibody to a conserved region of a protein of the invention. Antibodies having specificity for a protein of the invention may also be raised from fusion proteins.

Conventional methods can be used to prepare the antibodies. For example, by using a peptide of a protein of the invention, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)]. Hybridoma cells can be screened immunochemically for production of antibodies specifically reactive with the peptide and the monoclonal antibodies can be isolated. Therefore, the invention also contemplates hybridoma cells secreting monoclonal antibodies with specificity for a protein of the invention.

The term “antibody” as used herein is intended to include fragments thereof which also specifically react with a protein, of the invention, or peptide thereof. Antibodies can be fragmented using conventional techniques and the fragments screened for utility in the same manner as described above. For example, F(ab′)2 fragments can be generated by treating antibody with pepsin. The resulting F(ab′)2 fragment can be treated to reduce disulfide bridges to produce Fab′ fragments.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes the gene product of the genes of the psb cluster of the invention (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, reactive against proteins of the invention may also be generated by screening expression libraries encoding immunoglobulin genes, or portions thereof, expressed in bacteria with peptides produced from the nucleic acid molecules of the present invention. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). In an embodiment of the invention, antibodies that bind to an epitope of a protein of the invention are engineered using the procedures described in N. Tout and J. Lam (Clinc. Diagn. Lab. Immunol. Vol. 4(2):147-155, 1997).

The antibodies may be labelled with a detectable marker including various enzymes, fluorescent materials, luminescent materials and radioactive materials. Examples of suitable enzymes include horseradish peroxidase, biotin, alkaline phosphatase, O-galactosidase, or acetylcholinesterase; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include S-35, Cu-64, Ga-67, Zr-89, Ru-97, Tc-99m, Rh-105, Pd-109, In-111, I-123, 1-125, I131, Re-186, Au-198, Au-199, Pb-203, At-211, Pb-212 and Bi-212. The antibodies may also be labelled or conjugated to one partner of a ligand binding pair. Representative examples include avidin-biotin and riboflavin-riboflavin binding protein. Methods for conjugating or labelling the antibodies discussed above with the representative labels set forth above may be readily accomplished using conventional techniques.

The antibodies reactive against proteins of the invention (e.g. enzyme conjugates or labeled derivatives) may be used to detect a protein of the invention in various samples, for example they may be used in any known immunoassays which rely on the binding interaction between an antigenic determinant of a protein of the invention and the antibodies. Examples of such assays are radioimmunoassays, enzyme immunoassays (e.g. ELISA), immunofluorescence, immunoprecipitation, latex agglutination, hemagglutination, and histochemical tests. Thus, the antibodies may be used to identify or quantify the amount of a protein of the invention in a sample in order to diagnose P. aeruginosa infections.

A sample may be tested for the presence or absence of P. aeruginosa serotypes O1 to O20 by contacting the sample with an antibody specific for an epitope of PsbM (WbpM) or PsbN (WbpN) which antibody is capable of being detected after it becomes bound to PsbM (WbpM) or PsbN (WbpN) in the sample, and assaying for antibody bound to PsbM (WbpM) or PsbN (WbpN) in the sample, or unreacted antibody. A sample may also be tested for the presence or absence of P. aeruginosa serotypes O2, O5, O16, O18, and O20 by contacting the sample with an antibody specific for an epitope of a Rol, PsbA, PsbB, PsbC, PsbD, PsbE, Rfc, PsbF, PsbG, PsbH, PsbI, PsbJ, PsbK (also known as Wzz, WbpA, WbpB, WbpC, WbpD, WbpE, Wzy, WbpF, WbpG, WbpH, WbpI, WbpJ, WbpK respectively), HisH or HisF, protein which antibody is capable of being detected after it becomes bound to the protein in the sample, and assaying for antibody bound to protein in the sample, or unreacted antibody.

In a method of the invention a predetermined amount of a sample or concentrated sample is mixed with antibody or labelled antibody. The amount of antibody used in the process is dependent upon the labelling agent chosen. The resulting protein bound to antibody or labelled antibody may be isolated by conventional isolation techniques, for example, salting out, chromatography, electrophoresis, gel filtration, fractionation, absorption, polyacrylamide gel electrophoresis, agglutination, or combinations thereof.

The sample or antibody may be insolubilized, for example, the sample or antibody can be reacted using known methods with a suitable carrier. Examples of suitable carriers are Sepharose or agarose beads. When an insolubilized sample or antibody is used protein bound to antibody or unreacted antibody is isolated by washing. For example, when the sample is blotted onto a nitrocellulose membrane, the antibody bound to a protein of the invention is separated from the unreacted antibody by washing with a buffer, for example, phosphate buffered saline (PBS) with bovine serum albumin (BSA).

When labelled antibody is used, the presence of a P. aeruginosa serotype can be determined by measuring the amount of labelled antibody bound to a protein of the invention in the sample or of the unreacted labelled antibody. The appropriate method of measuring the labelled material is dependent upon the labelling agent.

When unlabelled antibody is used in the method of the invention, the presence of a P. aeruginosa serotype can be determined by measuring the amount of antibody bound to the P. aeruginosa serotype using substances that interact specifically with the antibody to cause agglutination or precipitation. In particular, labelled antibody against an antibody specific for a protein of the invention, can be added to the reaction mixture. The presence of a P. aeruginosa serotype can be determined by a suitable method from among the already described techniques depending on the type of labelling agent. The antibody against an antibody specific for a protein of the invention can be prepared and labelled by conventional procedures known in the art which have been described herein. The antibody against an antibody specific for a protein of the invention may be a species specific anti-immunoglobulin antibody or monoclonal antibody, for example, goat anti-rabbit antibody may be used to detect rabbit antibody specific for a protein of the invention.

The reagents suitable for applying the methods of the invention may be packaged into convenient kits providing the necessary materials, packaged into suitable containers. Such kits may include all the reagents required to detect a P. aeruginosa serotype in a sample by means of the methods described herein, and optionally suitable supports useful in performing the methods of the invention.

In one embodiment of the invention the kit contains a nucleotide probe which hybridizes with a nucleic acid molecule of the invention, reagents required for hybridization of the nucleotide probe with the nucleic acid molecule, and directions for its use. In another embodiment of the invention the kit includes antibodies of the invention and reagents required for binding of the antibody to a protein specific for a P.aeruginosa serotype in a sample. In still another embodiment of the invention, the kit includes primers which are capable of amplifying a nucleic acid molecule of the invention or a predetermined oligonucleotide fragment thereof, all the reagents required to produce the amplified nucleic acid molecule or predetermined fragment thereof in the polymerase chain reaction, and means for assaying the amplified sequences.

The methods and kits of the present invention have many practical applications. For example, the methods and kits of the present invention may be used to detect a P. aeruginosa serotype in any medical or veterinary sample suspected of containing P. aeruginosa. Samples which may be tested include bodily materials such as blood, urine, tissues and the like. Typically the sample is a clinical specimen from wound, bum and urinary tract infections. In addition to human samples, samples may be taken from mammals such as non-human primates, etc. Further, water and food samples and other environmental samples and industrial wastes may be tested.

Before testing a sample in accordance with the methods described herein, the sample may be concentrated using techniques known in the art, such as centrifugation and filtration. For the hybridization and/or PCR-based methods described herein, nucleic acids may be extracted from cell extracts of the test sample using techniques known in the art.

Substances that Affect O-antigen Synthesis and Assembly

A protein of the invention may also be used to assay for a substance which affects O-antigen synthesis or assembly in P. aeruginosa Accordingly, the invention provides a method for assaying for a substance that affects O-antigen synthesis or assembly in P. aeruginosa comprising mixing a protein of the invention with a test substance which is suspected of affecting the expression or activity of the protein, and determining the effect of the substance by comparing to a control.

In an embodiment of the invention the protein is an enzyme, and a method is provided for assaying for a substance that affects O-antigen synthesis and assembly in P. aeruginosa comprising incubating a protein of the invention with a substrate of the protein, and a test substance which is suspected of affecting the activity of the protein, and determining the effect of the substance by comparing to a control.

In a preferred embodiment the protein is PsbM which has dehydrogenase activity. Representative substrates which may be used with PsbM in the assay are precursor sugars such as glucose. Dehydrogenase activity may be assayed using conventional methods.

Compositions and Methods of Treatment

The substances identified by the methods described herein, antisense nucleic acid molecules, and antibodies, may be used for modulating one or both of O-antigen synthesis and assembly in P. aeruginosa and accordingly may be used in the treatment of infections caused by P.aeruginosa. O-antigen is a virulence factor of P. aeruginosa and it is responsible for serum resistance. Therefore, substances which can target LPS biosynthesis in P. aeruginosa to change the organism into making “rough” LPS devoid of the long chain O-antigen (B-band) polymers will be useful in rendering the bacterium susceptible to attack by host defense mechanisms. The substances identified by the methods described herein, antisense nucelic acid molecules, and antibodies are preferably used to treat infections caused by P. aeuginosa serotypes 02, 05, 16, 18 and 20. The substances etc. are also preferably used to treat infections caused by P. aeruginosa serotypes 03 or 06 which are predominant clinical isolates. It will be appreciated that the substances may also be useful to treat infections caused by other members of the family Pseudomonadaceae (eg. P. cepacia and P. pseudomallei), and to treat other bacteria which produce O-antigen, (e.g. other gram negative bacteria such as E. coli, S. enterica, Vibrio cholera, Yersinia entercolitica and Shigella flexneri).

The substances identified using the methods described herein may be formulated into pharmaceutical compositions for adminstration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions which may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The reagents suitable for applying the methods of the invention to identify substances that affect O-antigen synthesis and assembly in P. aeruginosa may be packaged into convenient kits providing the necessary materials packaged into suitable containers. The kits may also include suitable supports useful in performing the methods of the invention.

The utility of the substances, antibodies, and compositions of the invention may be confirmed in experimental model systems.

The invention will be more fully understood by reference to the following examples. However, the examples are merely intended to illustrate embodiments of the invention and are not to be construed to limit the scope of the invention.

### EXAMPLES

Materials and methods used in Examples 1 to 3 described herein include the following:

Bacterial Strains and Culture Conditions

The bacterial strains used in this study are listed in Table 6. All bacterial strains were maintained on Tryptic Soy Agar (Difco Laboratories, Detroit, Mich.). P. Isolation Agar (PIA; Difco) was used for selection of transconjugants following mating experiments. Antibiotics used in selection media include: ampicillin at 100 μg/ml for E. coli and carbenicillin at 450 μg/ml for P. aeruginosa, tetracycline at 15 μg/ml for E. coli and 90 μg/ml for P. aeruginosa (250 μg/ml in PIA), gentamicin at 10 μg/ml for E. coli and 300 μg/ml for P. aeruginosa.

DNA Procedures

Small-scale preparation of plasmid DNA was done utilizing the alkaline lysis method of Birnboim and Doly (1979). Large-scale preparations of plasmid DNA were obtained using the Qiagen midi plasmid kit (Qiagen Inc., Chatsworth, Calif.), according to procedures specified by the manufacturer. Whole genomic DNA was isolated from P. aeruginosa following the method of Goldberg and Ohman (1984). Restriction enzymes were purchased from GIBCO/BRL and Boehringer-Mannheim (Mannheim, Germany). T4 DNA ligase, T4 DNA polymerase and alkaline phosphatase were purchased from Boehringer-Mannheim. All enzymes were used following suppliers' recommendations. DNA was transformed into E. coli and P. aeruginosa by electroporation using a Bio-Rad electroporation unit (Bio-Rad Laboratories; Richmond, Calif.) and according to the protocols supplied by the manufacturer. Electrocompetent cells of E. coli and P. aeruginosa were prepared according to the methods of Binotto et al. (1991) and Farinha and Kropinski (1990), respectively. Recombinant plasmids were mobilized from E. coli DH5α to P. aeruginosa through triparental matings as described by Ruvkun and Ausubel (1981). Plasmids were also mobilized from E. coli SM10 to P. aeruginosa using the method of Simon et al. (1983). Genomic DNA was transferred to Zetaprobe membrane (Bio-Rad) by capillary transfer following the manufacturer's instructions. Southern hybridizations were done at 42° C. for 18-24h with DNA previously labelled with dUTP conjugated to digoxigenin (DIG) (Boehringer-Mannheim). Labelling of DNA was done according to the manufacturer's recommendations. Hybridized DNA was detected using an anti-DIG polyclonal antibody conjugated to alkaline phosphatase and AMPPD (0.235 mM 3-(2′-Spiroadamantane)-4-methoxy-4(3″-phosphoryloxy)-phenyl-1,2-dioxetane) (Boehringer-Mannheim), followed by exposure to X-ray film (E. I. Du Pont de Nemours & Co., Wilmington, Del.).

Tn1000 Mutagenesis of pFV.TK6

Tn1000 mutagenesis of pFV.TK6 was performed as described previously (Lightfoot and Lam, 1993) using the method of de Lencastre et al. (1983).

DNA Sequencing

DNA sequence analysis of the 1.9 kb insert of pFV.TK8 was performed by the MOBIX facility (McMaster University, Hamilton ON). The 1.9 kb XhoI-HindIII insert of pFV.TK8 was cloned into the sequencing vector pBluescript II KS and double-strand sequenced using a model 373A DNA sequencing unit (Applied Biosystems, Foster City, Calif.). Oligodeoxynucleotide primers for sequencing were synthesized on an Applied Biosystems model 391 DNA synthesizer and purified according to the manufacturers' instructions. The Taq DyeDeoxy™ Terminator Cycle Sequencing Kit (Applied Biosystems) was used for cycle sequencing reactions which were carried out in an Ericomp (San Diego, Calif.) model TCX15 thermal cycler.

Sequence Analysis

The computer software programs Gene Runner for Windows (Hastings Software, New York, N.Y.) and PCGENE (IntelliGenetics, Mountain View, Calif.) were used for nucleic acid sequence analysis, amino acid sequence analysis, and characterization of the predicted protein. DNA and protein database searches were performed using the NCBI BLAST network server (Altschul et al., 1990; Gish and States, 1993).

Mutagenesis of the rfc Gene of P. aeruginosa PAO1

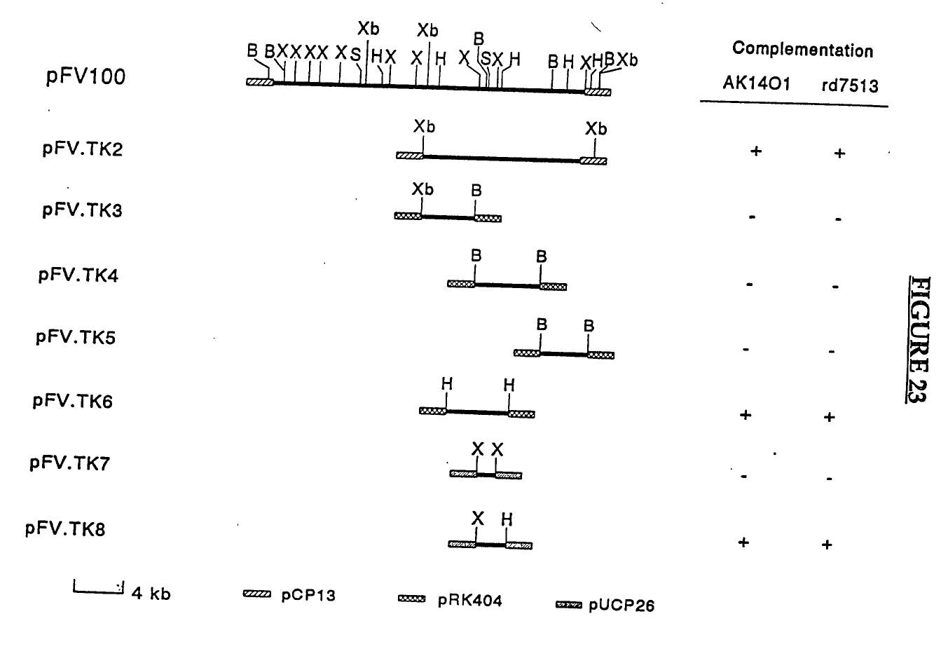

In order to construct P. aeruginosa rfc chromosomal mutants a novel gene replacement vector, pEX100T (Schweizer and Hoang , 1995) was used. This vector, called pEX100T, contains the sacB gene of B. subtilis which imparts sucrose sensitivity on gram-negative organisms and allows for positive selection of true mutants from the more frequently occurring merodiploids. In the first step of this experiment, the 5.6 kb HindIII fragment of pFV.TK6 was blunt-ended using T4 DNA polymerase and subcloned into the SmaI site of pEX100T. An 875 bp Gm^{R} cassette from pUCGM (Schweizer, 1993) was then cloned into the single BamHI site of the insert DNA. The resulting plasmid, pFV.TK9, was transformed into the mobilizer strain E. coli SM10 and then conjugally transferred into PAO1 (Simon et al., 1983). After mating, cells were plated on PIA containing 300 μg/ml of Gm. Colonies that grew on the Gm-containing medium were picked and streaked on PIA containing 300 μg/ml Gm and 5% sucrose to identify isolates that had lost the vector-associated sacB gene, and thus had become resistant to sucrose. Southern blot analysis was performed to verify that gene replacement had occurred (FIG. 24).

Preparation of LPS

LPS used in sodium dodecyl sulfate-polyacrylamide gel electrophoresis (SDS-PAGE) and Western immunoblotting experiments was prepared according to the proteinase K digest method of Hitchcock and Brown (1983).

SDS-PAGE

The discontinuous SDS-PAGE procedure of Hancock and Carey (1979) utilizing 15% running gels was used. LPS separated by SDS-PAGE was visualized by silver-staining according to the method of Dubray and Bezard (1982).

Immunoblotting

The Western immunoblotting procedure of Burnette (1981) was used with the following modifications. Nitrocellulose blots were blocked with 3% (w/v) skim milk followed by incubation with hybridoma culture supernatant containing either MAb MF15-4, specific for O5 LPS, or MAb NlFlO, specific for A-band LPS. The blots were developed at room temperature, using goat anti-mouse F(ab′)₂ fragment conjugated antibody (Jackson Immunoresearch Laboratories, West Grove, PA) and a substrate consisting of 30 mg of Nitro Blue Tetrazolium and 15 mg of 5-bromo-4-chloro-3-indolyl phosphate toluidine (Sigma, St. Louis, Mo.) in 100 ml of 0.1 M bicarbonate buffer (pH 9.8).

### Example 1

Analysis of the LPS from mutants AK14O1 and rd7513. Strain AK14O1 has been previously shown to contain A-band LPS; its B-band LPS consists of complete core plus one O-repeat unit (SR phenotype) (Berry and Kropinski, 1986; Lam et al., 1992). Strain rd7513 is a mutant of AK14O1 that has the SR phenotype but is no longer producing A-band LPS, due to a mutation in an A-band biosynthetic gene (Lightfoot and Lam, 1991). Strain rd7513 was used in this study described in the examples, in addition to AK14O1; but the majority of this investigation will focus on AK14O1.

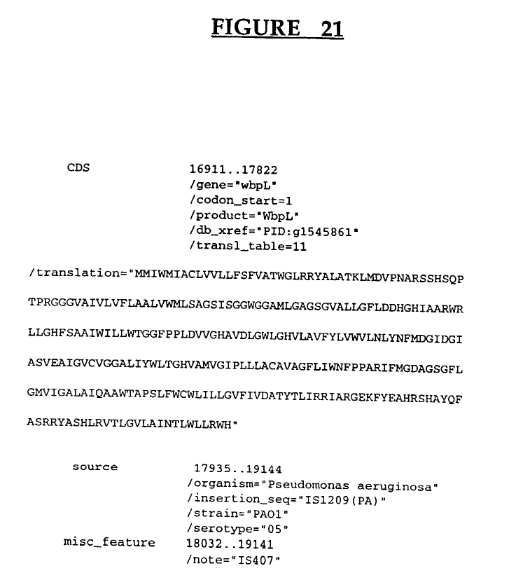

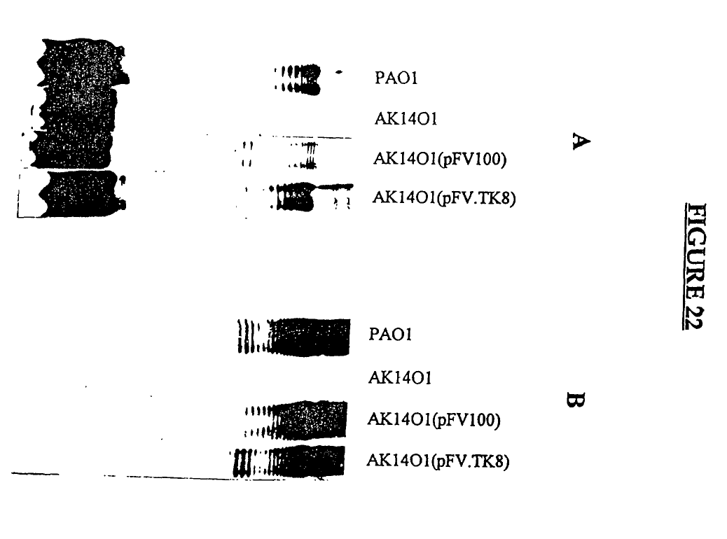

Complementation of O-antigen expression in P. aeruginosa AK14O1. Mobilization of pFV100, which contains the O5 rfb gene cluster, into SR mutant AK14O1 resulted in production of O5 B-band LPS. These results suggest that an O-polymerase gene might be localized on the cloned DNA. Analysis of LPS isolated from PAO1 and AK14O1(pFV100) in both silver-stained SDS-PAGE gels and Western immunoblots, reacted with O5-specific MAb MF15-4, revealed that the two strains expressed similar high molecular weight LPS profiles (FIG. 22 a, b). In order to localize the putative rfc gene on the 26 kb insert of pFV100, various subclones were made (FIG. 23) and used in complementation studies with AK14O1. Plasmid pFV.TK², which contains a 16.5 kb XbaI fragment from pFV100 was able to complement O-antigen production after mobilization into AK14O1 (data not shown). Plasmids pFV.TK3, pFV.TK4, and pFV.TK5 were generated and mobilized into AK14O1, however none of the three plasmids was able to complement B-band synthesis in this mutant. Subsequently, pFV.TK6 which contains a 5.6 kb HindIII insert was made and was able to complement the SR phenotype of AK14O1 (data not shown).

Transposon Tn1000 mutagenesis of pFV.TK6. Transposon mutagenesis using Tn1000 was performed in order to more precisely define the region of insert DNA in pFV.TK6 responsible for complementation of O-antigen expression in AK14O1. pFV.TK6::Tn1000 recombinants were mobilized into AK14O1 and then screened for the lack of expression of O-antigen using O-specific MAb MF15-4. Plasmid DNA was isolated from colonies that did not react with MAb MF15-4, and subjected to restriction enzyme analysis to determine the location of the Tn1000 insertion in pFV.TK6. Three Tn1000 insertions in a 1.5 kb XhoI fragment were found to interrupt O-antigen expression in AK14O1 (FIG. 23). This 1.5 kb XhoI fragment was cloned into vector pUCP26 (pFV.TK7) and mobilized into AK14O1. In Western immunoblots of LPS from AK14O1(pFV.TK7) with MAb MF15-4 no reaction of this antibody with high molecular weight B-band LPS could be detected (data not shown). Therefore, the 1.5 kb XhoI insert in pFV.TK7 was unable to restore the O-polymerase- function in AK14O1. A 1.9 kb XhoI-HindIII fragment was then subcloned into pUCP26 and the resulting plasmid was designated pFV.TK8 (FIG. 23). Mobilization of this recombinant plasmid into both SR mutants, AK14O1 and rd7513, resulted in restoration of O-antigen expression. Silver-stained SDS-PAGE gels and Western blots reacted with MAb MF15-4, showed that the AK14O1(pFV.TK8) transconjugants expressed levels of O5 B-band LPS comparable to that produced by the wild-type PAO1 (FIG. 22).

Southern analysis using a 1.5 kb XhoI probe. The 1.5 kb XhoI insert of pFV.TK7, internal to the rfc coding region, was labelled with dUTP conjugated to digoxigenin and used to probe XhoI-digested chromosomal DNA from the twenty P. aeruginosa serotypes. The probe hybridized to a 1.5 kb fragment in serotypes O2, O5, O16, O18, and O20 (data not shown), suggesting that these serotypes may share a similar O-polymerase gene. These hybrization results are not surprising in that serotypes 02, 05, 016, and O20 share a similar O-repeat backbone structure (Knirel, 1990). Although the O-antigen structure of serotype O18 has not yet been determined, it exhibits cross-reactivity with polyclonal antisera raised against serotype O5 (data not shown), suggesting that it has an O-repeat unit structure similar to that of O5. In a recent study, Collins and Hackett (1991) found that a probe generated from the rfc gene of S. enterica (typhimurium) cross-hybridized to chromosomal DNA of Salmonella groups A, B, and D1 strains but not with strains of groups D2 or E2, suggesting that the former may share a common rfc gene. In addition, studies done by Nurminen and coworkers (1971) have shown that the O-polymerase enzymes of Salmonella groups B and D1 strains are able to polymerize O-repeat units of either serotype.

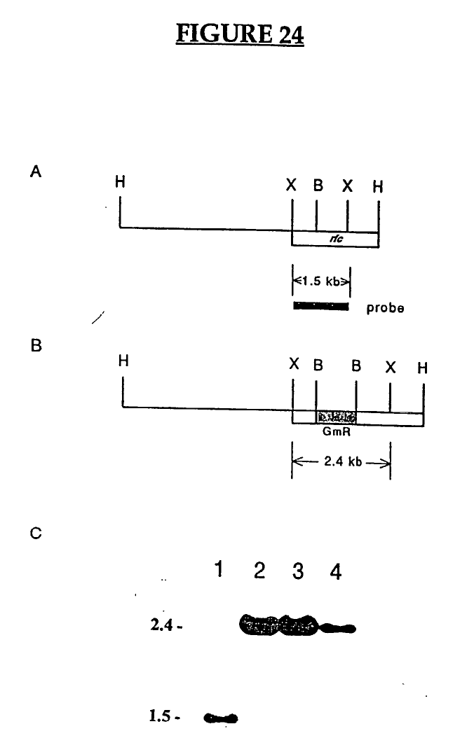

Generation of P. aeruginosa chromosomal rfc-mutants. In order to confirm that the insert DNA of pFV.TK8 codes for an O-polymerase gene, insertional mutagenesis was performed and the resulting plasmid used for homologous recombination with the PAO1 chromosome. In the first step, the 5.6 kb insert of plasmid pFV.TK6 was cloned into a novel gene replacement vector, pEX100T, (Schweizer and Hoang, 1995). pEX100T is a pUC19-based plasmid that does not replicate in P. aeruginosa; therefore, maintenance of plasmid DNA can only occur after homologous recombination into the chromosome. The 5.6 kb insert of pFV.TK6 was used for gene replacement instead of the 1.9 kb insert of pFV.TK8 to ensure that there was sufficient DNA for homologous recombination. The next step involved insertion of an 875 bp Gm^{R} cassette into a unique BamHI site in the insert DNA (FIG. 24b). This step generated a mutation in the rfc gene and provided a means of later selecting for colonies that had undergone homologous recombination. Because the vector, pEX100T, contains the sacB gene of Bacillus subtilis it renders Gram-negative organisms sensitive to sucrose. Streaking Gm^{R} recombinants on media containing 5% sucrose allowed separation of true recombinants from merodiploids, since merodiploids exhibit sucrose-sensitivity because of the presence of the vector-associated sacB gene. Of the eighty Gm^{R} colonies that were isolated, twenty-four were found to be sucrose-resistant. Three of the twenty-four isolates were randomly chosen for further characterization and were designated OP5.2, OP5.3, and OP5.5. Southern blot analysis of chromosomal DNA from these three putative mutants was performed in order to confirm that gene replacement had occurred. The 1.5 kb XhoI fragment of pFV.TK8 was used to probe XhoI-digested chromosomal DNA isolated from the PAOI wild-type strain as well as OP5.2. OP5.3, and OP5.5. In strains that had undergone gene replacement, XhoI digestion should yield a probe-hybridizable fragment of 2.4 kb instead of 1.5 kb because of the insertion of the 875 bp Gm^{R} cassette (FIG. 24 a, b). Southern blot analysis of the three Gm^{R}, sucrose-resistant isolates revealed a probe-reactive fragment of 2.4 kb (FIG. 24 c, lanes 2-4); whereas, the probe reacted with a 1.5 kb fragment of the PAO1 control DNA (FIG. 24 c, lane 1), demonstrating that gene replacement had occurred in OP5.2, OP5.3, and OP5.5. Analysis of LPS from these three strains in silver-stained gels and Western immunoblots with O5-specific MAb MF15-4 demonstrated that they were not capable of producing long chain B-band O-antigen (FIG. 25a, b). Immunoblots reacted with A-band specific MAb NlFlO revealed that, like the SR mutant AK14O1, these three mutants were still producing A-band LPS (FIG. 25c). Biosynthesis of A-band LPS therefore, appears to be unaffected by this chromosomal mutation. The relative mobility of the core-lipid A bands was also similar to that of the SR mutant AK14O1 (FIG. 25a); therefore the LPS phenotype of the three rfc knockout mutants was identical to that of AK14O1. Mobilization of pFV.TK8 into OP5.2, OP5.3 and OP5.5 restored O-antigen expression in the three mutants (data not shown), indicating that the PAO1 chromosomal modification was the result of a direct mutation of the rfc gene and not caused by a secondary mutation.

Nucleotide sequence determination and analysis of rfc. The 1.9 kb XhoI-HindIII insert of pFV.TK8, containing the rfc coding region, was cloned into pBluescript and subjected to double-strand nucleotide sequence analysis. Examination of the nucleotide sequence (FIG. 9; GenBank accession number U17294) revealed one open reading frame (ORF) that coded for a protein of 438 amino acids, with a predicted mass of 48.9 kDal. This ORF was designated ORF48.9.

Analysis of the P. aeruginosa rfc mol. % G+C content (44.8%; Table 6) revealed that it is significantly lower than that of the rest of the genome (67.2%; Palleroni, 1984). A low G+C content is a common feature of reported rfc genes (Collins and Hackett, 1991; Brown et al., 1992; Klena and Schnaitman, 1993; Morona et al., 1994) and has also been observed in all of the rfb clusters so far analyzed. The finding that the gene coding for the O-polymerase enzyme and the genes encoding the O-antigen repeat units have a compatible G+C content is not surprising since the specificity of the enzyme must relate to the structure of it substrate.

Homology searches of both the nucleotide and the amino acid sequences of the P. aeruginosa rfc gene were performed using EMBL/GenBank/PDB and Swiss-PROT (release 28.0) databases (Altschul et al., 1990; Gish and States, 1993). Comparison of the P. aeruginosa rfc sequences with sequences reported for other prokaryotic genes revealed no significant homology, including with those reported for other rfc genes. Previous studies on the structure of P. aeruginosa O-antigens have revealed that their sugar compositions differ significantly from most other enterobacterial O-antigens (Knirel et al., 1988). Neutral sugars, which are commonly found in enteric O-antigens, are only rarely found in O-antigens of P. aeruginosa. In addition, P. aeruginosa O-antigens are rich in amino sugars, many of which are substituted with acyl groups, a phenomenon rarely found in natural carbohydrates. Given the unique sugar composition of P. aeruginosa O-antigens, and the finding by Morona et al. (1994) that the S. flexneri Rfc protein showed no homology with other enteric Rfc proteins, it is not surprising that the P. aeruginosa Rfc protein exhibited no sequence homology with those of other enteric organisms.

The P. aeruginosa rfc gene product does, however, have several features in common with other reported Rfc proteins, including the fact that it is very hydrophobic. The mean hydropathic index of the P. aeruginosa Rfc is 0.8 while those of other enteric organisms have been reported to range from 0.65-1.08 (Table 7). Examination of the hydropathy profile of this protein and analysis of the amino acid sequence, using the software program PCGENE, revealed that it is an integral membrane protein with 11 putative membrane-spanning domains (Klein et al., 1985). The Rfc proteins of S. enterica (typhimurium) and S. enterica (muenchen) are reported to have 11 membrane-spanning domains, while that of S. flexneri is reported to have 13 (Morona et al., 1994); therefore, structural similarities appear to exist among the Rfc proteins of these four organisms.

Codon usage and amino acid composition analysis. When the codon usage and amino acid composition of the P. aeruginosa Rfc protein was compared with that reported for S. enterica (typhimurium), S. enterica (muenchen), and Shigella flexneri Rfc proteins (Collins and Hackett, 1991; Brown et al., 1992; Morona et al., 1994), significant similarities were found between them (data not shown). Rfc proteins have been reported to contain a high content of three amino acids, namely, leucine, isoleucine, and phenylalanine (Morona et al., 1994). These three amino acids account for 27, 30, and 37 % of the total amino acids of the Rfc proteins of S. enterica (typhimurium), S. enterica (muenchen), and Shigella flexneri, respectively (Morona et al., 1994). In the Rfc protein of P. aeruginosa, these amino acids represent 30% of the total amino acid composition.

In summary, the present inventors have isolated an rfc gene in P. aeruginosa O5 encoding an O-polymerase enzyme. Using a gene-replacement system, P. aeruginosa rfc-chromosomal mutants were generated which expressed the typical sr lps phenotype. The P. aeruginosa Rfc is similar to other reported Rfc proteins in that it is very hydrophobic, containing 11 membrane-spanning domains; the Rfc coding region has a lower mol. % G+C than the P. aeruginosa chromosomal average; and it has a similar amino acid composition and codon usage to that reported for other Rfc proteins.

### Example 2

Isolation of a Rol Gene in P. aeruginosa 05 (PA01) Encoding a Protein which Regulates O-antigen Chain Length

The P. aeruginosa serotype 05 (PA01) rol gene (regulator of O-chain length) was cloned from a genomic DNA cosmid library. An open reading frame (ORF) of 1046 bp, encoding a 39.3 kDa protein, was identified. The characterization of the function of Rol was facilitated by the generation of knockout mutants.

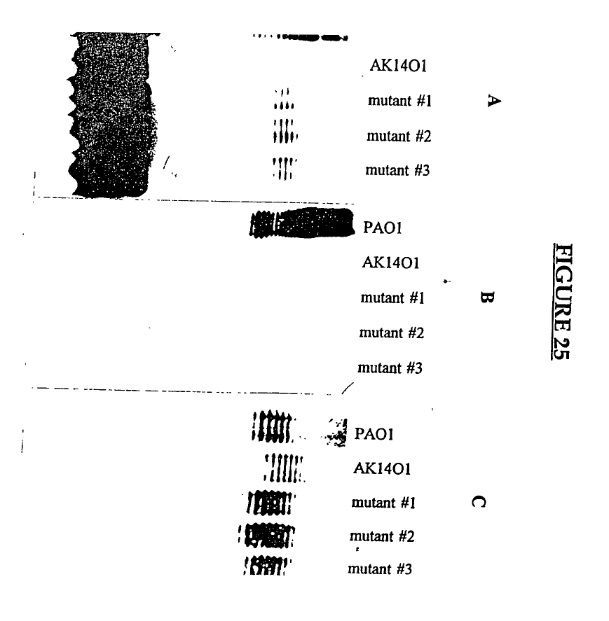

The DNA sequence of a subclone of pFV100, pFV161 (FIG. 26), was found to have homology to the rol genes from a number of members of the family Enterobacteriaceae. However, only the 3′ end of the putative rol gene was present on pFV161. A cosmid library of P. aeruginosa (PA01) genomic DNA was screened using a digoxigenin-labled probe from pFV161 to identify an overlapping cosmid (pFV400) containing the complete rol gene. Southern blot analysis of DNA from pFV400, digested with a number of different restriction enzymes, was performed. The pFV161 probe hybridized to an approximately 2.3 kb HindIII fragment of pFV400. Assuming the rol gene of P. aeruginosa serotype 05 (PA01) was similar in size (approx. 1 kb) to members of the family Enterobacteriaceae (Morona et al., 1995), this fragment would be sufficient to contain the entire putative rol gene. This 2.3 kb HindIII fragment was subcloned into the vector pBluescript II SK (PDI Biosciences, Aurora, Ontario, Canada) and named pFV401 (FIG. 26).

Nucleotide sequencing of the 2.3 kb HindIII insert was performed using dye terminator cycle sequencing (GenAlyTiC sequencing facility, University of Guelph), and an open reading frame (ORF) that coded for a protein of 348 amino acids, with a predicted mass of 39.3 kDA, was identified (GenBank accession #U50397). Homology searches using the GenBank database through the NCBI Blast network server were performed (Altschul et al., 1990; Gish and States, 1993). Both the nucleotide and the deduced amino acid sequences of the putative P. aeruginosa rol gene showed approximately 33-35% amino acid homology between the putative Rol protein and the Rol proteins of Salmonella enterica serovar typhimurium, Escherichia coli, and Shigella flexneri (Morona et al., 1995) (Table 5).

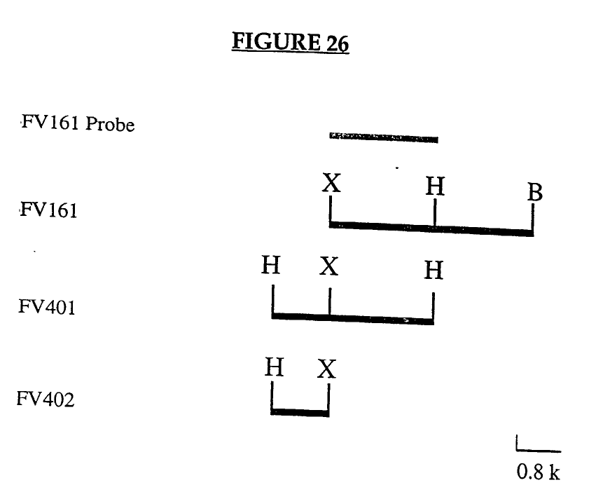

To confirm that the insert DNA of pFV401 codes for a Rol protein, insertional mutagenesis was performed and the resulting plasmid construct used for homologous recombination with the PA01 chromosome. Briefly, the 2.3 kb insert of pFV401 was cloned into a novel gene-replacement vector, pEX100T (Schweizer and Hoang, 1995), that does not replicate in P. aeruginosa. pEX100T also contains the sacB gene of B. subtills which imparts sucrose sensitivity on Gram-negative organisms and allows for positive selection of true mutants from the more frequently occurring merodiploids. Next, an 875 bp gentamicin-resistance (GM^{R}) cassette from pUCGM (Schweizer, 1993) was inserted into a unique XhoI site in the insert DNA. The resulting plasmid (pFV401TG) was transformed into the mobilizer strain E. coli SM10 and then conjugally transferred into PA01 (Simon et al., 1983). After mating, cells were plated on P. isolation agar (PIA; Difco Laboratories, Detroit, Mich.) containing 300 μg ml⁻¹ gentamicin (Sigma Chemical Co., St. Louis, Mo.) and 5% sucrose. This selective medium allows the identification of isolates that have undergone homologous recombination and lost the vector-associated sacB gene thus, becoming resistant to sucrose. Southern blot analysis with both wild-type rol gene and Gm^{R} cassette probes was used to confirm the insertional mutation. The wild-type control and the mutants showed probe reactive fragments of 2.3 kb and 3.1 kb respectively (FIG. 27).

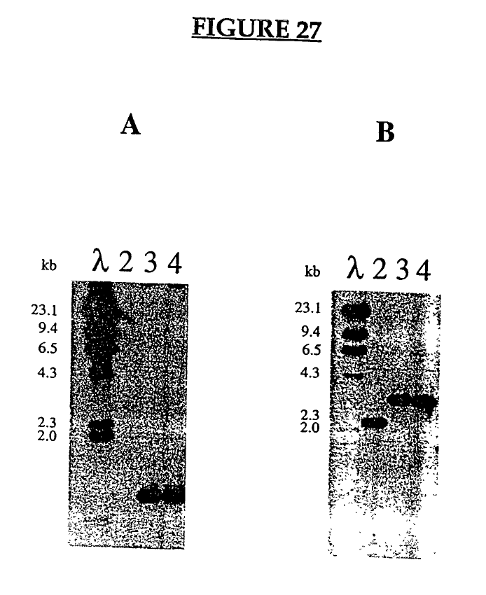

The LPS of the mutants was prepared according to the proteinase K digest method of Hitchcock and Brown (1983). The LPS was analyzed using sodium dodecyl sulfate polyacrylamide gel electrophoresis (SDS-PAGE) and Western immunoblots according to the methods described previously (de Kievit et al., 1995). When compared with the wild-type strain, the mutant LPS showed a marked alteration in the O-antigen ladder-like banding pattern, in which there was a decrease in high molecular weight bands and an increase in visible low molecular weight bands. This change corresponds to a loss of bimodal distribution in O-antigen length (FIG. 28).

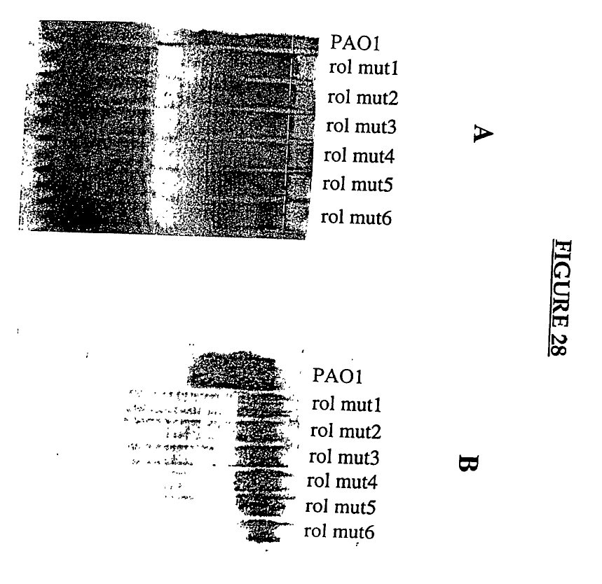

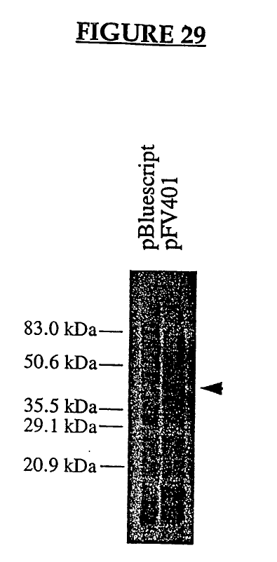

A T7 expression system (Tabor and Richardson, 1985) was used for expression of the Rol protein. A unique protein band with an apparent molecular mass of 39 kDa was observed. This expressed polypeptide corresponded well to the predicted mass of 39.3 kDa. This band was not observed in the vector-only control (FIG. 29).

In conclusion, a rol gene was isolated in P. aeruginosa 05 (PA01) encoding a protein which regulates O-antigen chain length. Using a gene-replacement system, P. aeruginosa rol::Gm^{R} knockout mutants were generated which express LPS with unregulated O-antigen chain length. Thus, the P. aeruginosa 05 (PA01) Rol protein has both sequence and functional homology to other reported Rol proteins. This also confirms that the pathway for P. aeruginosa B-band LPS biosynthesis is Rfc-dependent. The function of Rol is often associated with the Rfc protein, an O-polymerase (Whitfield, 1995, Kievit et al., 1995).

### Example 3

Sequencing of the psb Gene Cluster.

The isolation of a cosmid clone, pFV100, containing the psb gene cluster of P. aeruginosa O5 identified in accordance with the present invention, was previously described (Lightfoot and Lam, 1993). Several subclones of pFV100 containing the psb genes were constructed. The sequencing and characterization of two of these clones (pFV111 and pFV110), containing the rfc and psbL (rfbA) genes respectively, has previously been described (de Kievit et al., 1995; Dasgupta and Lam, 1995). Sequencing of the remainder of the pFV100 insert was undertaken in order to identify all the genes required for synthesis of the O5 O-antigen.

Sequencing of the entire insert of pFV100, a total of 24416 bp, revealed a large number of open reading frames (ORFs) on both strands. ORFs which were reading in the same direction as rfc and psbL and which had homology either to any previously identified polysaccharide or antibiotic biosynthetic genes or to highly conserved bacterial genes were characterized further. A total of 21 ORFs which could be involved in synthesis of the O5 O-antigen were identified (Table 1). These genes were designated psbA through psbN in the 5′ to 3′ direction, with the exceptions of rol and rfc, which were named according to convention. A further 4 ORFs with high homology to other bacterial genes or insertion sequences but which are not thought to be involved with LPS synthesis were identified (hisH, hisF, uvrB, IS407; Table 1).

Distribution of the psb Genes Among the 20 Serotypes of P. aeruginosa and Localization of the O5-Specific Region.

Southern blot analysis of the 20 serotypes of P. aeruginosa using various psb genes as probes revealed an interesting dichotomy. All of the probes tested which were 5′ to the IS407 element hybridized only with chromosomal DNA from serotypes O2, O5, O16, O18 and O20 (Table 1). As stated above, these five serotypes have biochemically and structurally similar O-antigens (FIG. 1). Although the O-antigens of serotypes O2, O5, O16, O18, and O20 are serologically distinct and have been shown to have clear biochemical differences, none of the psb genes tested hybridized only to serotype O5 chromosomal DNA at high stringency.

In contrast with these findings, probes for DNA sequences 3′ to the IS407 element, and the IS407 element itself, hybridized with the chromosomal DNA from all 20 serotypes of P. aeruginosa (Table 1). These results show that the insertion sequence is the junction between the portion of the psb cluster specific for O5 and related serotypes (hereinafter referred to as the O5-specific region, or sometimes as the Group I genes) and the non-specific chromosomal DNA. Therefore, psbL appears to be the last gene of the O5-specific region. Despite the fact that the DNA 3′ of the insertion element is not O5-specific, this region is thought to contain at least two ORFs (psbM and psbN or sometimes referred to as the Group II genes) which may be involved in O5 LPS biosynthesis (see below).

A 1.2 kb probe from the extreme 5′ end of the insert of pFV100 hybridized only to the five related serotypes, indicating that the 5′ end of the O5-specific region had not been cloned. This probe was used to isolate an overlapping cosmid, pFV400. Various subclones of pFV400 were constructed to localize the 5′ end of the O5-specific region to within a 1.3 kb SstI-XhoI fragment located 1.7 kb upstream of the 5′ end of pFV100. Preliminary sequence analysis of this upstream region revealed no additional ORFs thought to be involved with LPS synthesis. Also, no insertion sequences could be found in this region of DNA. Localization of the 5′ end of the O5-specific region to the 1.3 kb SstI-XhoI fragment means the total amount of DNA which is specific to O5 and related serotypes is approximately 20 kb.

The Composition and Chromosomal Milieu of the O5 psb Cluster.

The %G+C of the P. aeruginosa chromosome has been determined by various methods to be approximately 65-67% (Palleroni, 1984; West and Iglewski, 19XX). The %G+C content of the P. aeruginosa O5 psb cluster within the O5-specific region averages 51.1% overall, with individual genes ranging from a low of 44.5% (psbG) to a high of 56.8% (psbK) (Table 1). These results are consistent with those seen for other rfb genes, averaging at least 10% below the chromosomal background, and this is thought to be reflective either of origin in a low %G+C background (Reeves, 1993) or of possible regulatory constraints (Collins and Hackett, 1991; Morona et al., 1994a). The %G+C content of the psbM and psbN genes, which fall outside the O5-specific region, averages 62.6 %.

Sequence analysis of pFV100/pFV400 revealed no homology to gnd (encoding 6-phosphogluconate dehydrogenase) in the regions flanking the LPS genes. However, P. aeruginosa has been shown to convert glucose-6-phosphate to 6-phosphogluconate as part of the Entner-Douderoff pathway, suggesting a homologue of the gnd gene is located elsewhere on the chromosome. The location of the P. aeruginosa his operon is not known, but the few his auxotrophic lesions that have been mapped on the chromosome of serotype O5 (strain PAO1) are several minutes from the A- and B-band LPS clusters (Lightfoot and Lam, 1993; Hollaway et al., 1994). Interestingly, two his genes (hisH and hisF) were found, in the middle of the psb cluster, within the O5-specific region (see below). Because these genes fail to hybridize with all twenty serotypes of P. aeruginosa at high stringency, it is likely they are not native P. his genes, but were acquired along with the psb genes in a horizontal transfer event.

Homology searches of the Genbank databases with each of the ORFs in the psb cluster were performed. Assignment of putative function for the products of the ORFs was made based on homology of the encoded proteins to those previously described. Because the O-antigen of P. aeruginosa O5 contains two similar 2,3-diacetaminido-mannuronic acid residues, it is anticipated that both residues share a common biosynthetic pathway.

The 5′ End of the pFV100 Insert Contains a Partial Rol Gene.

The partial open reading frame at the 5′ end of the insert of pFV100 was found to have low homology at the amino acid level (34-37%) with the Rol proteins of Escherichia coli (Batchelor et al., 1992; Bastin et al., 1993), Salmonella enterica sv Typhimurium (Batchelor et al., 1992; Bastin et al., 1993), and Shigella flexneri (Morona et al., 1994b). Only 479 bp of rol-homologous DNA (encoding 159 amino acids) were present from the XhoI cloning site of pFV100. This sequence represented approximately the 3′ half of the putative rol gene, based on the sizes of previously described rol genes. Using the partial gene as a probe, the entire rol gene has been cloned from an overlapping cosmid, pFV400, and its function confirmed by mutational analysis (Example 2). In other Rfc-dependent LPS gene clusters, the rol gene is positioned near or at the end of the cluster. These results, along with the large number of ORFs already identified on pFV100 suggested that most, if not all, of the genes required for O5 O-antigen biosynthesis are present on this cosmid. psbA.

There is a distance of 807 bases between the rol gene and the first adjacent gene, psbA. Although P. aeruginosa promoters are not well defined, there are similarities with E. coli promoters (Harley and Reynolds, 1987; Deretic et al., 1989). There is a possible σ⁷⁰-like promoter sequence and a putative ribosomal binding site (RBS) located 93 bp and 7 bp, respectively, upstream of the start of psbA (FIG. 31). PsbA has homology (summarized in Table 2) to EpsD, thought to be a dehydrogenase required for synthesis of exopolysaccharide in Burkholderia solanaceraeum (Huang and Schell, 1995); to VipA, involved in synthesis of the Vi antigen in S. enterica sv Typhi (Hashimoto et al., 1993); and to RffD, a UDP-N-acetyl-D-mannosaminuronic acid dehydrogenase involved in synthesis of Enterobacterial Common Antigen (ECA) in E. coli (Meier-Dieter et al., 1992). ECA is an exopolysaccharide common to most enterics that can be linked to lipid A-core in rough strains. It is composed of N-acetyl-D-glucosamine (GlcNAc), N-acetyl-D-mannosaminuronic acid (ManNAcA), and 4-acetamido-4, 6-dideoxy-D-galactose (Fuc4NAc).

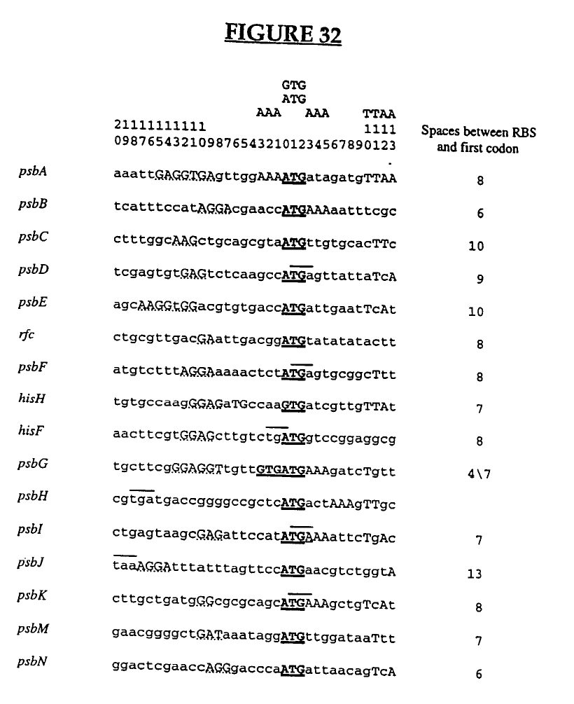

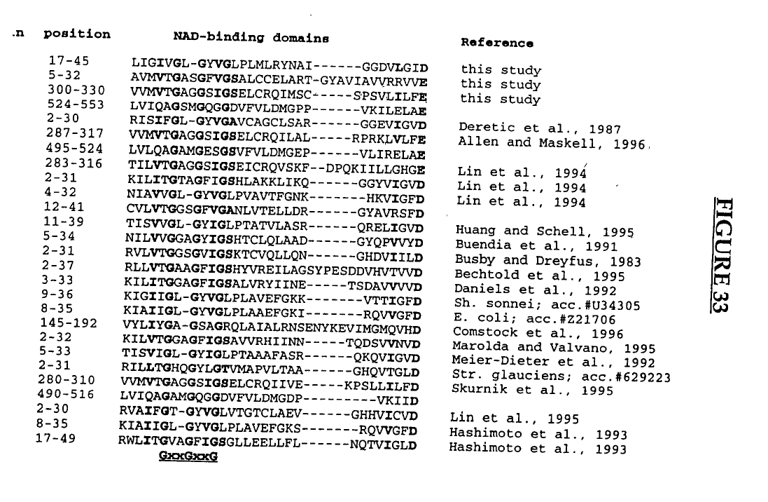

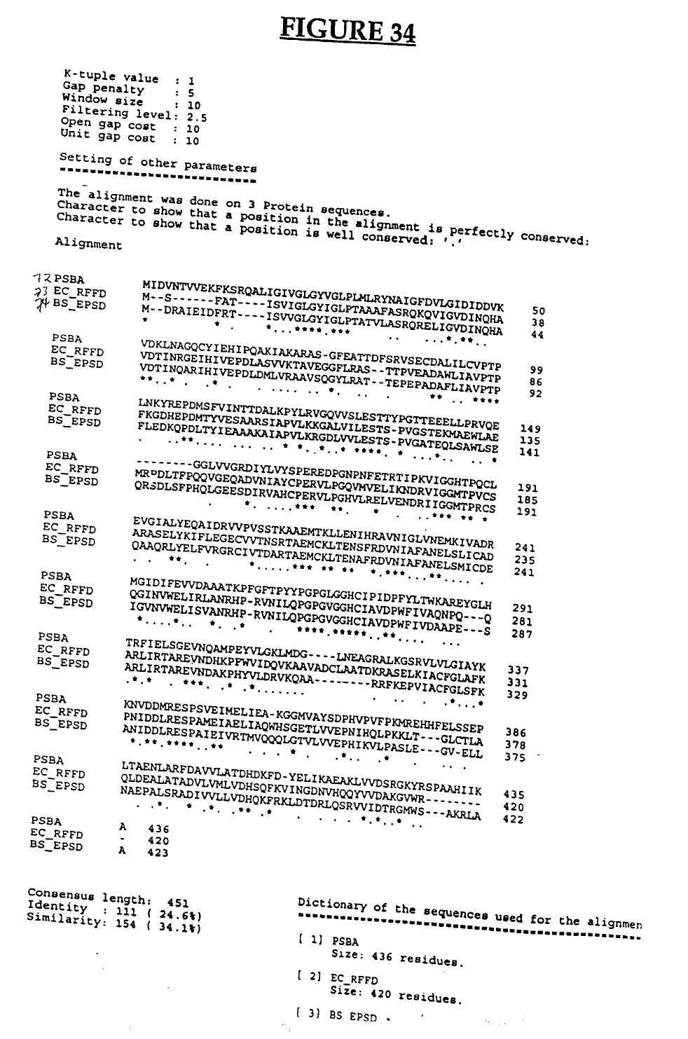

PsbA also has homology with CapL, involved in type 1 capsular polysaccharide production in Staphylococcus aureus (Lin et al., 1994). The type 1 capsule is composed of taurine, 2-acetamido-2-deoxy-fucose (Fuc2NAc) and 2-acetamido-2-D-galacturonic acid (Gal2NAcA). The sugar composition of both ECA and type 1 capsule are similar to the P. aeruginosa O5 O-antigen. PsbA also has a low level of homology with ORF7 of the Vi antigen region of E. coli/Citrobacter freundii (accession #Z21706), and several GDP-mannose and UDP-glucose dehydrogenases, including AlgD of P. aeruginosa (Deretic et al., 1987). AlgD is a GDP-mannose dehydrogenase required for alginate synthesis. These homologies suggest that PsbA functions as a dehydrogenase involved in the biosynthesis of the mannuronic acid residues, possibly converting UDP-N-acetyl-D-mannosamine into UDP-N-acetyl-D-mannosaminuronic acid. A large number of dehydrogenases including PsbA (as well as PsbK and PsbM, below) contain a consensus nicotinamide adenosine dinucleotide (NAD)-binding domain, thought to be important for activity (FIG. 33). An alignment of the amino acid sequences of some PsbA-like proteins is shown in FIG. 34. psbB.

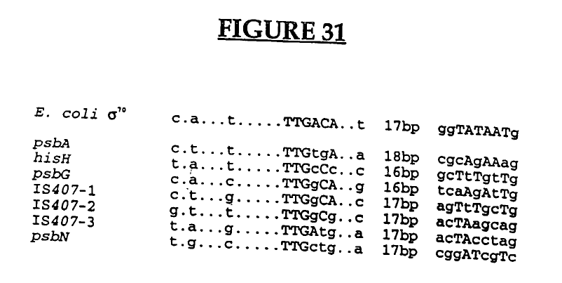

The psbB gene start is 74 bases from the termination codon of psbA, but no separate promoter sequence for psbB could be detected. A putative RBS is located 6 bp from the initiation codon for psbB and the second codon is AAA, the preferred second codon in E. coli (Gold and Stormo, 1987; FIG. 32). The psbB gene product is possibly an oxido-reductase, dehydratase, or dehydrogenase. It is 28.2% homologous to the LmbZ protein of Streptomyces lincolnesis required for lincomycin production (Peschke et al., 1995), and also has homology with the pur10 gene product of Streptomyces alboniger required for puromycin production (Tercero et al., 1996). PsbB has 17% homology to the BplA protein from B. pertussis required for LPS production (Allen and Maskell, 1996) and even weaker homology to ORF334 and MocA from Rhizobium meliloti found in the operon for rhizopine catabolism (Rossbach et al., 1994). In B. pertussis, the BplA protein is thought to catalyze the final step in the biosynthesis of UDP-diNAcManA from UDP-diNAcMan (Allen and Maskell, 1996).

Several of the psb genes were found to have high homology with bpl genes, suggesting a common ancestry. B. pertussis has semi-rough LPS, with only one O-antigen unit attached to the core oligosaccharide. The composition of the B. pertussis O-antigen unit is N-acetylglucosamine (GlcNAc), 2,3-dideoxy-2,3-N-acetylmannosaminuronic acid (2,3-diNAcManA), and N-acetyl-N-methyl fucosamine (FucNAcMe) (Allen and Maskell, 1996). These sugars are similar to those comprising ECA, S. aureus type 1 capsule, and the P. aeruginosa O5 O-antigen. The amino acid homology between PsbB and BplA as well as the similarties in O-antigen unit composition suggest that PsbB could have a homologous function to that of BplA. Unlike the other putative dehydrogenases encoded in the psb cluster, PsbB does not contain a consensus NAD-binding domain.

psbC.

The start of psbC overlaps significantly (343 bases) with the stop of psbB, and psbC could encode a large protein of 85.3 kDa (766 amino acids). Careful scrutiny of the DNA sequencing results confirmed no sequencing errors were present. Protein expression will determine whether this entire large ORF is translated. The large size of this protein may indicate it resulted from a fusion event. There is a weak potential RBS upstream of the AUG codon of psbC (FIG. 32).

The carboxy-terminal portion of PsbC has homology with a hypothetical protein (HI0392) derived from the Haemophilus influenzae genome sequence (Fleischmann et al., 1995). HI0392 is a 245 amino acid protein of unknown function, with several hydrophobic domains, and is thought to be an integral membrane protein. There is homology between PsbC and the macrolide 3-O-acyltransferase acyA gene from the Streptomyces thermotolerans carbomycin biosynthetic cluster (Arisawa et al., 1995). PsbC also has weak homology with ExoZ of R. meliloti, involved in succinoglycan production (Buendia et al., 1991), and with NodX of R. leguminosarum, involved in nodulation (Davis et al., 1988). ExoZ is a 317 amino acid protein, also with multiple hydrophobic domains, while NodX is a 367 amino acid protein thought to be located in the cytoplasmic membrane. ExoZ and NodX genes are both putative 3-O-acyltransferases. A summary of the homologies between the above proteins is shown in Table 2. The similarities indicate PsbC, particularly the carboxy terminal portion, may have 3-O-acyltransferase activity, and could be involved in acetylation of the mannuronic acid residues in the O5 O-antigen.

psbD.

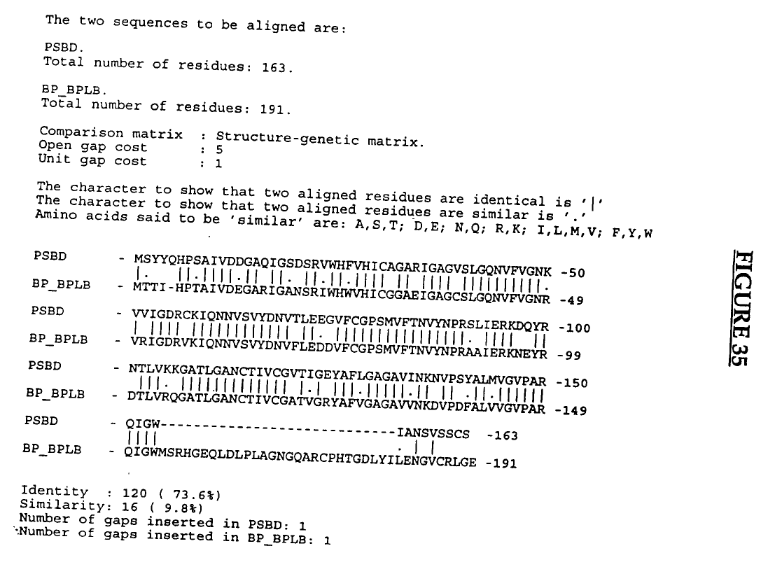

The psbD gene appears to be translationally coupled with the psbC gene, since its start codon overlaps the stop codon of psbC. A potential RBS is located 9 bp upstream of the psbD AUG codon (FIG. 32). The product of the psbD gene is most homologous with the product of the bplB gene in the B. pertussis LPS biosynthetic cluster (Allen and Maskell, 1996). PsbD and BplB appear to be O-acetyl transferases, and have some homology to serine O-acetyl transferases (CysE) from a variety of bacteria, including Buchnera aphidicola (Lai and Baumann, 1992), Bacillus stearothermophilus (Gagnon et al., 1994), B. subtilis (Ogasawata et al., 1994), E. coli (Denk and Bock, 1987), S. enterica sv Typhimurium (accession #P29847), H. influenzae (Fleischmann et al., 1995), and the plant Arabidopsis thaliana (Bogdanova et al., 1995) (Table 2, FIG. 35). As with PsbC, PsbD is probably involved in the acetylation of the mannuronic acid residues comprising two-thirds of the O5 repeat unit. While bplA and bplB are contiguous on the B. pertussis chromosome, the psb homologues, psbB and psbD respectively, are separated by the large psbC gene.

psbE.

psbE has high homology with a B. pertussis LPS biosynthetic gene, bplC. psbD and psbE are adjacent to one another in the psb cluster, as are bplB and bplC in the bpl cluster (Allen and Maskell, 1996). However, they do not appear to be translationally coupled, since there are 86 bases between the end of psbD and the start of psbE. While there is a potential RBS 9 bp before the psbE start (FIG. 32), it is not known whether this gene can be transcribed from a promoter internal to the psbD gene. There are some sequences with weak homology to the E. coli consensus promoter sequence in that area.

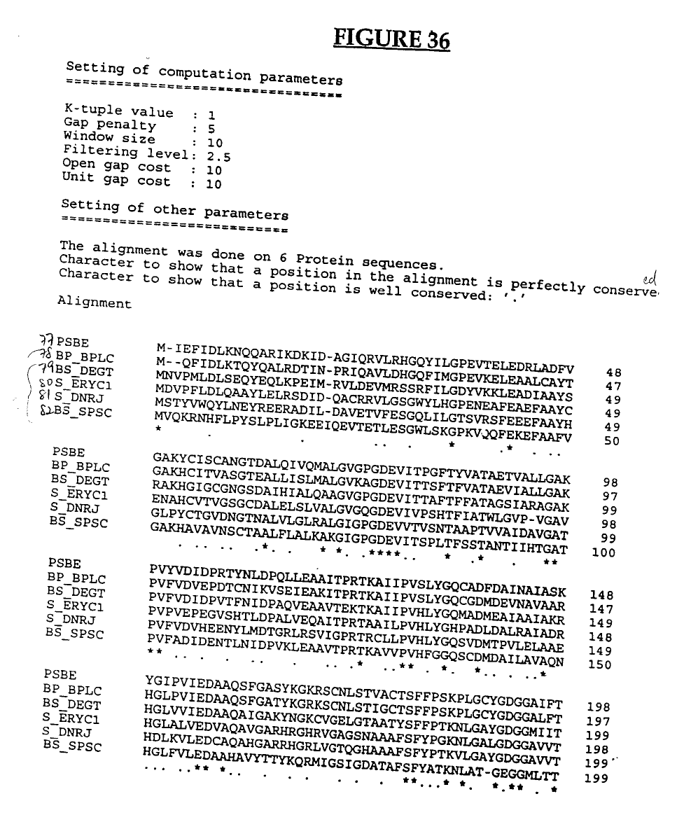

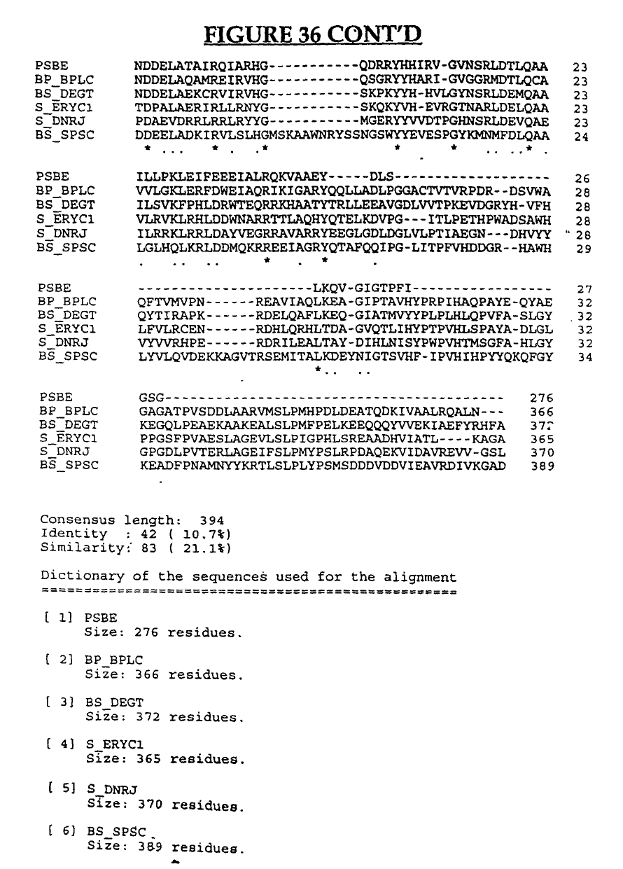

Also homologous to PsbE are DegT, from B. subtilis (Takagi et al., 1990), Saccharopolyspora erythraea ErbS (ERYCL) involved in erythromycin synthesis (Dhillon et al., 1989), DnrJ from Streptomyces peucetius required for daunorubicin biosynthesis (Stutzman et al., 1992) and SpsC from B. subtilis involved in spore coat polysaccharide biosynthesis (Glaser et al., 1993) (summarized in Table 2). There is also weak homology between PsbE and both MosB for rhizopine synthesis in R. meliloti (Murphy et al., 1993) and Yiff, a hypothetical protein in the rffE/rffT intragenic region of E. coli (Daniels et al., 1992). The proteins DegT/DnrJ/ERYC1/SpsC form a family of proteins formerly thought to form the DNA-binding component of sensory-transduction two-component regulatory systems. More recently, however, their function is suggested to be in the biosynthesis of 2,3-, 2,4-, and 2,6-dideoxy sugars such as the 2,3-dideoxy mannuronic acid produced by P. aeruginosa O5 (Thorsen et al., 1993). An alignment of the amino acid sequences of the PsbE-like proteins is shown in FIG. 36.

The O-antigen Polymerase, rfc.

The rfc gene starts 254 bases downstream of the end of the psbE gene. This gene was cloned, sequenced and characterized as described in Example 1. Knockout mutations generated by insertion of a gentamicin cassette into rfc were used to confirm this gene encoded the O-antigen polymerase. Gentamicin-resistant mutants were shown to have the semi-rough phenotype (See Example 1) characteristic of an rfc mutant (Mäkelä and Stocker, 1984).

psbF.

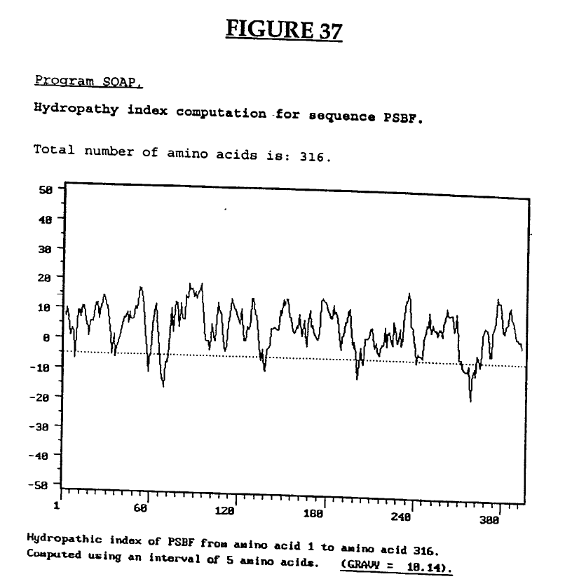

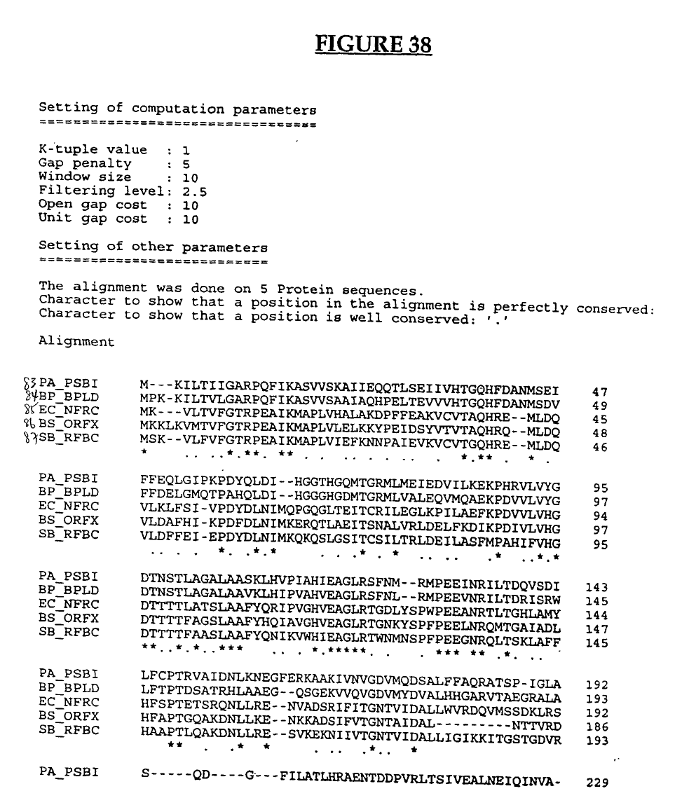

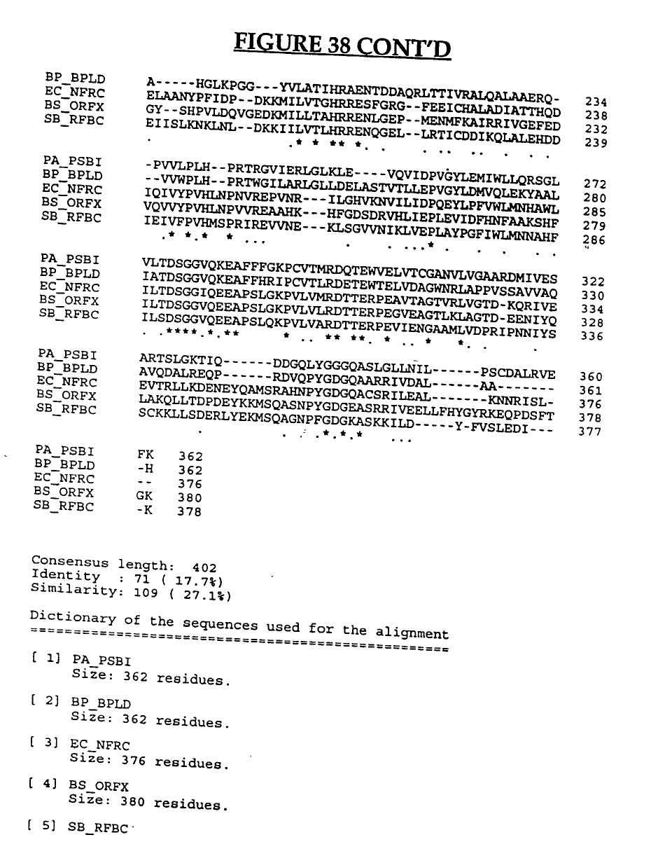

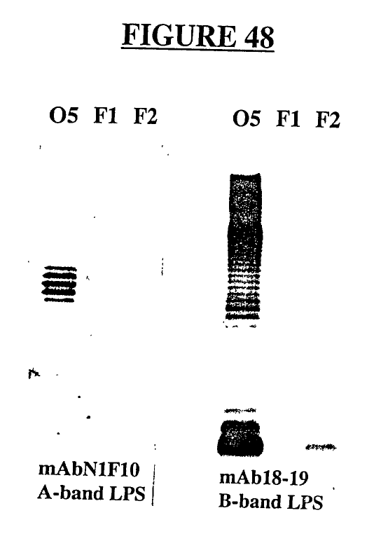

The psbF gene appears to be translationally coupled with the rfc gene since they have an overlapping stop and start. There is a RBS sequence 8 bp upstream of the initiation codon of psbF. It is most homologous, to the ExoT protein of R. meliloti (Glucksmann et al., 1993), which is thought to be involved in succinoglycan transport. There is also a small amount of homology to FeuC of B. subtilis, part of its iron uptake system (Quirk et al., 1994). PsbF is the most hydrophobic protein encoded by the psb cluster (Table 1) and has 9-10 membrane-spanning domains. This secondary structure is remniscent of that of RfbX, the putative flippase found in Rfc-dependent O-antigen clusters (FIG. 37) (Schnaitman and Klena, 1993). Mutations in RfbX have been found to be unstable and deleterious to the host strain (Schnaitman and Klena, 1993). Recently Liu et al. (1996) confirmed that RfbX (Wzx) mutants accumulate one O-antigen unit on undecaprenol on the inside of the cytoplasmic membrane. PsbF knockout mutants generated by insertion of a gentamicin resistance cassette into psbF are both A and B-band minus (FIG. 48). PsbF may be the P. aeruginosa O5 equivalent of RfbX.

The hisH and hisF Genes.

The histidine operon, containing genes required for the biosynthesis of the amino acid histidine, has previously been shown to lie adjacent to the rfb clusters of several enteric species (reviewed in Schnaitman and Klena, 1993). Comparison of the chromosomal map locations of the P. aeruginosa O5 A- and B-band LPS clusters with those of known PAO1 his mutations showed there were no his genes located adjacent to either the psa (11-13 min) or psb (37 min) clusters (Lightfoot and Lam, 1993; Holloway et al., 1994). Therefore, the identification of two genes with high homology to the genes hisF and hisH of various bacterial species in the middle of the psb cluster was unexpected. The hisH and hisF genes are located between the psbF and psbG genes (FIG. 1), and transcribed in the same direction. The direction of transcription of the his genes in previously characterized rfb clusters is opposite to that of the rfb genes (Ames and Hartman, 1974; Macpherson et al., 1994).

While the deduced amino acid sequence of hisF appears to give a complete open reading frame (from bases 10387 to 11142), the sequence of hisH appears to be lacking an AUG initiation codon at the location predicted for the start of the protein based on amino acid homology. However, there are potential starts at three GUG codons located 51, 72, and 132 bp upstream of the first AUG, located at base 9830. The size of the protein corresponding to the product of hisH is approximately 21 kDa, indicating it is probably translated from either of these putative starts. Only the GUG codon at 9777 is preceded by a good RBS (FIG. 32); none of the other potential start codons have consensus RBS sites. N-terminal analysis of the HisH product will confirm the translational start.

Protein expression analysis of this region shows the products of these genes are expressed in vitro in both orientations, indicating there is a promoter region preceding the his genes that can be recognized by E. coli. Analysis of the sequence upstream of the putative start sites of hisH shows there is a potential promoter sequence with partial homology to the E. coli consensus −35 and −10 regions (FIG. 31). This homology is within the range seen in previously reported P. aeruginosa promoter sequences that can function in E. coli (Deretic et al., 1989; Ronald et al., 1992).

In K. pneumoniae, the products of the hisH and hisF genes have been shown to form a heterodimeric enzyme complex required for the conversion of N′-[(5′phosphoribulosyl)-formimino]-5-aminoimidazole-4-carboxamide-ribonucleotide (5′-PRFAR) to imidazole glycerol-phosphate (IGP) and 5′-phosphoribosyl-4-carboxamide-5-aminoimidazole (ZMP) (Rieder et al., 1994). Although the products of the hisH and hisf genes have been shown to function together, the hisH and hisF genes themselves are separated by a third gene, hisA (Alifano et al., 1996). The hisA and hisH genes are highly related and are thought to have arisen through gene duplication. The gene order of hisHAF has been found in all bacterial species characterized to date (Alifano et al., 1996).

Comparison of the amino acid sequence homologies of various HisF and HisH proteins (Tables 3 and 4) showed that the P. aeruginosa psb HisF and HisH proteins are not closely related to any of the HisF/HisH proteins characterized thus far. Comparisons of P. aeruginosa psb HisF with the other HisF proteins shown in Table 6 shows that it is the most distantly related protein of the group analyzed, at approximately 50% homology.

psbG.

There is a distance of 138 bp between hisF and psbG, and a putative promoter is identified in this region (FIG. 31). A RBS is identified 4 bp from a putative GUG start and 7 bp from the adjacent AUG start codon (FIG. 32). The optimum spacing of a RBS from the initiation site is 8±2 bp, suggesting the AUG codon is likely to be the start. PsbG has limited homology to ORF2 (11.2%) of Vibrio cholerae O-antigen (Comstock et al., 1996), and less homology with NfrB of H. influenzae, a formate-dependent nitrate reductase (Fleischmann et al., 1993), and Pfk, a phosphofructokinase of the Gram positive bacterium, Lactococcus lactis (Xiao and Moore, 1993). Interestingly, the homology is associated with NfrB centres around the metal binding recognition site CXXCH, of which there are five in NfrB and one in PsbG (amino acids 24-28).

Insertion of a gentamicin cassette into psbG results in B-band deficient mutants of PAO1, suggesting a role for it in O-antigen biosynthesis.

psbH.

There are 15 bp between psbG and psbH, however, no RBS can be detected upstream of the psbH start codon. The third codon is AAA (FIG. 32). PsbH demonstrates low homology with CapM (14.2%) of S. aureus (Lin et al., 1994), involved in the synthesis of N-acetogalactosamino uronic acid. PsbH also has homology with a number of glycosyl transferases, including IcsA (17.1%) (accession #U39810) and RfaK (13%) (accession #U35713) of Neisseria meningitidis, RfbF (11.3%) of Klebsiella pneumoniae (Keenleyside and Whitfield, 1994). There is also a low level of homology with RfpB of Shigella dysenteriae (Göhmann et al., 1994), and BplH and BplE of B. pertussis (Allen and Maskell, 1996). These enzymes are likely to belong to a family of transferases involved in the addition of a similar sugar to the growing O-antigen unit.

RfpB, RfaK, and RfbF are glucosyl- or galactosyl transferases and it is likely that CapM is the transferase involved in the addition of N-acetogalactosaminouronic acid. This suggests that PsbH is one of the two ManA transferases.

PsbH also has very limited homology to the DnaK proteins of R. meliloti (Falah and Gupta, 1994) and Agrobacterium tumefaciens (Segal and Ron, 1995). However, the homology is concentrated around the central region of PsbH. DnaK is a chaperonin, and is thought to have a role in gene regulation. Homology around the functional domain of DnaK may suggest a role for psbH/PsbH in regulation of the psb cluster.

psbI

The start codon of psbI overlaps the stop codon of psbH. A putative RBS is situated 6 bp upstream of the AUG start and the second codon is AAA (FIG. 32). PsbI demonstrates strong homology with BplD of B. pertussis (Allen and Maskell, 1996) (Table 2). BplD is purported to initiate the first step in the biosynthesis of 2,3-diNAcManA. PsbI also demonstrates moderate homology to NfrC and ORF o389 (RffD) of E. coli (Daniels et al., 1992), EpsC of Burkholderia solanacearum (Huang and Schell, 1995), YvyH of B. subtilis (Soldo et al., 1993) and RfbC of S. enterica sv Borreze (Keenleyside and Whitfield, 1995). EpsC is thought to be involved in the biosynthesis of N-acetylgalactosaminuronic acid, and RfbC is thought to be UDP-N-acetylglucosamine 2-epimerase. Alignment of PsbI and related proteins is shown in FIG. 10. Based on these homologies, it is likely that PsbI converts UDP-N-acetylglucosamine to UDP-N-acetylmannosamine as the first step in the biosynthesis of mannuronic acid. Interestingly, the genes encoding the remaining enzymes in this pathway are located upstream and somewhat removed from the psbI gene (psbABDE).

psbJ.

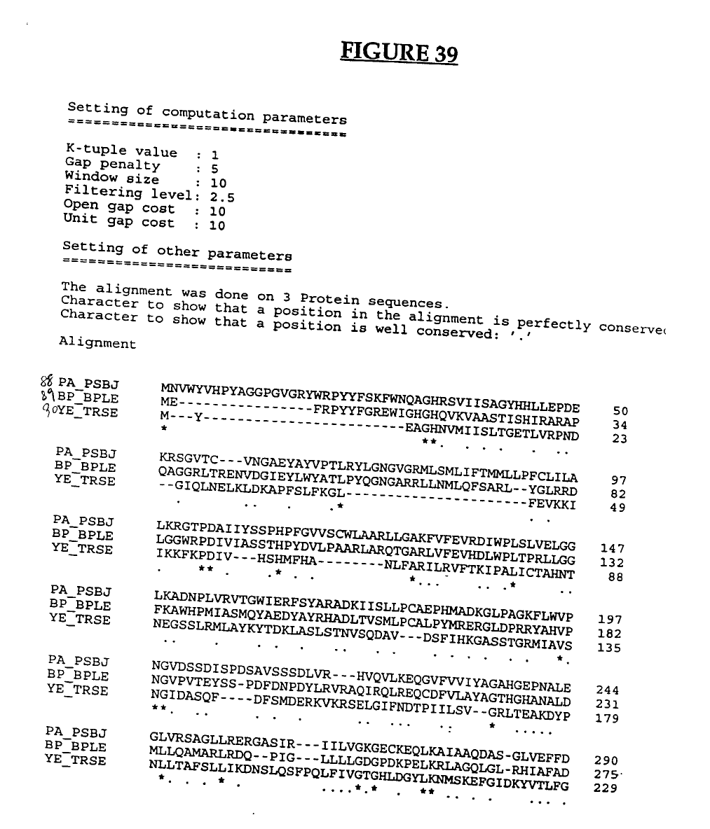

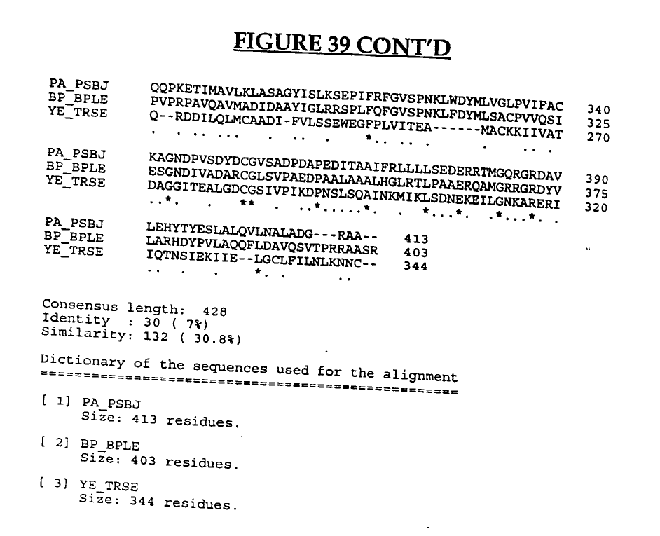

The distance between psbI and psbJ is 17 bp. A putative RBS is present immediately following the stop codon of psbI, 13 bp from the AUG start codon of psbJ (FIG. 4). PsbJ demonstrates reasonable homology to Bp1E (52.6%) of B. pertussis, a glycosyl transferase thought to attach either 2,3-diNAcManA or FucNAcMe to the O-unit (Allen and Maskell, 1996) (Table 2). TrsE of Yersinia enterocolitica also has homology to PsbJ (Skurnik et al., 1995), and is thought to be one of the galactosyl- or mannosyl transferases. An alignment of PsbJ and PsbJ-like proteins is shown in FIG. 39. As BplE also has limited homology with PsbH, it is likely that both PsbH and PsbJ are the transferases involved in the addition of the two mannuronic acid residues to the B-band O-antigen unit. PsbJ has two putative membrane-spanning domains at the N-terminus, and may be anchored in the cytoplasmic membrane.

psbK.

The start codon of psbK overlaps the stop codon of psbJ, and the second codon is AAA (FIG. 32). PsbK demonstrates homology to a series of glucose dehydratases, including StrP of Streptomyces glauciens involved in streptomycin biosynthesis (accession number 629223), ExoB of R. meliloti (Buendia et al., 1991), ORF o355 (incorrectly assigned RffE) of E. coli (Daniels et al, 1992, Macpherson et al., 1994), GraE of Streptomyces violaceoruben (Bechtold et al., 1995) and RfbB of a number of organisms including N. meningitidis (Hamerschmidt et al., 1994) and E. coli (Marolda and Valvano, 1995). Alignment of these proteins show the presence of an NAD-binding domain (GXXGXXG) near the N-terminal end (FIG. 5; Macpherson et al., 1994). RfbB and o355 are known to be involved in the biosynthesis of FucNAc (Meier-Dieter et al., 1992). Based on these homologies, PsbK is thought to be dTDP-D-glucose 4,6-dehydratase, required as the second step in the biosynthesis of FucNAc.

psbL.

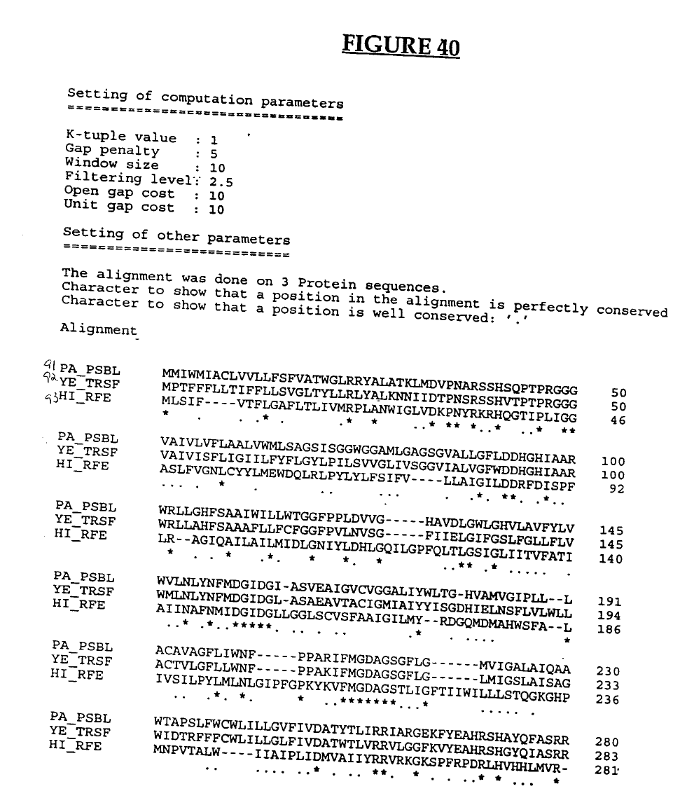

There are 59 bp between the end of psbK and the start of psbL but no RBS could be detected in the region preceding the double start codons (FIG. 32. Identification of the psbL(rfbA) gene has previously been reported (Dasgupta and Lam, 1995). Further characterization of PsbL suggests it functions as a transferase, and is thought to initiate O-antigen unit biosynthesis with the addition of FucNAc to undecaprenol, based on its homology to Rfe. The alignment of PsbL with TrsF from Y. enterocolitica (Skurnik et al., 1995) and Rfe from E. coli (Daniels et al., 1992) is shown in FIG. 40. Rfe is the initial transferase involved in the biosynthesis of ECA and some O-antigens (Schnaitman and Klena, 1993; Macpherson et al., 1994), transferring GlcNAc to undecaprenol (Meier-Dieter et al., 1992). Because the first transferase in the biosynthesis of O-antigen interacts with undecaprenol, it would be expected to be a hydrophobic protein. PsbL is the most hydrophobic (hydropathy index of 0.84, Table 1) of the three putative transferases encoded in the psb cluster (PsbH, PsbJ, PsbL).

IS407_{Pa}.

Following the psbL gene is an insertion sequence with 61.5% nucleotide identity with the previously characterized IS407 element of B. cepacia (Wood et al., 1991). This homology prompted the designation IS⁴⁰⁷_{Pa}, with the subscript _{Pa} to indicate it is the P. aeruginosa version. Both elements are similar in size (1243 bp for IS407_{Bc} and 1211 for IS407_{Pa}) and have very similar imperfect inverted repeats (IR) of 12 and 11 bp respectively. The IS407 elements are similar to IS sequences from other soil-, water- and plant-associated bacteria, including ISR1 from R. meliloti (Priefer et al., 1989), IS511 from Caulobacter crescentens, IS1222 from Enterobacter agglomerans, IS476 from Xanthamonas campestris (Kearney and Staskawicz, 1990), and IS911 from S. dysenteriae (Prére et al., 1990). There have been previous reports of IS elements in P. aeruginosa (Pritchard and Vasil, 1990; Sokol et al., 1994) but none of these have homology to the above group; therefore this is the first report of IS407 in P. aeruginosa. Southern blot analysis using the IS⁴⁰⁷_{Pa} as a probe showed it is present in all 20 serotypes of P. aeruginosa (Table 2), and most serotypes appear to have only a single copy of the element.

psbM.

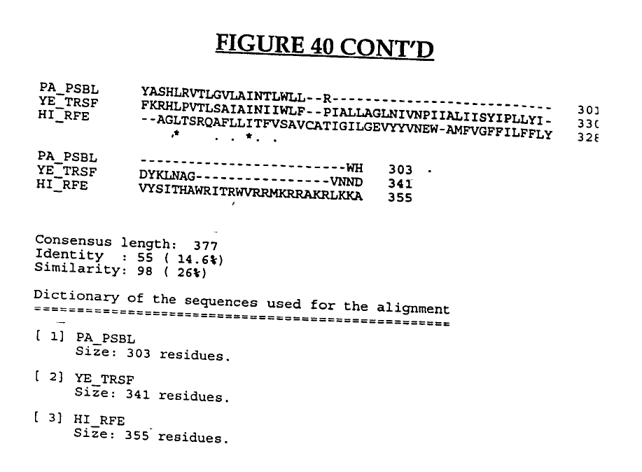

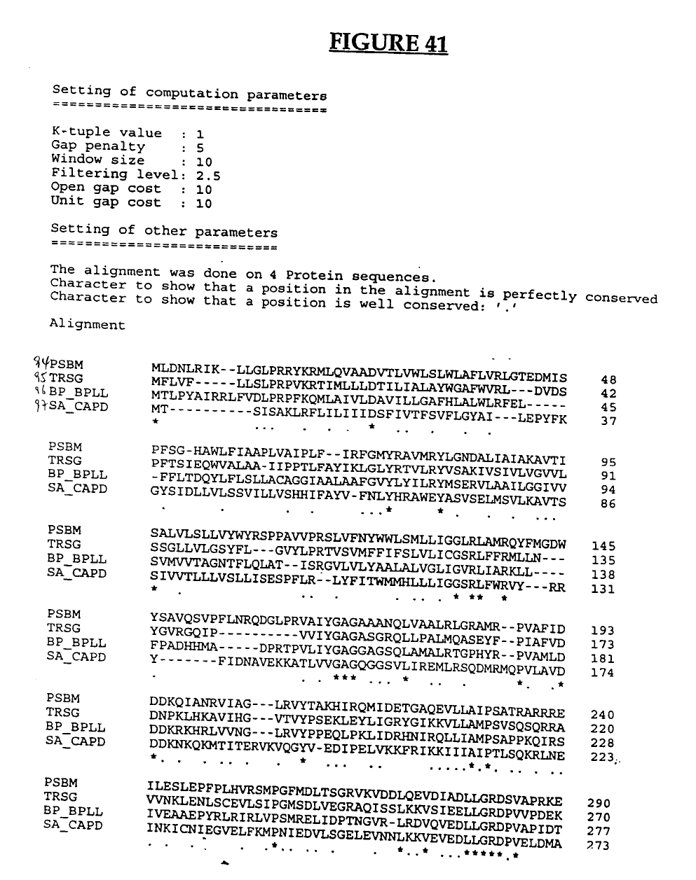

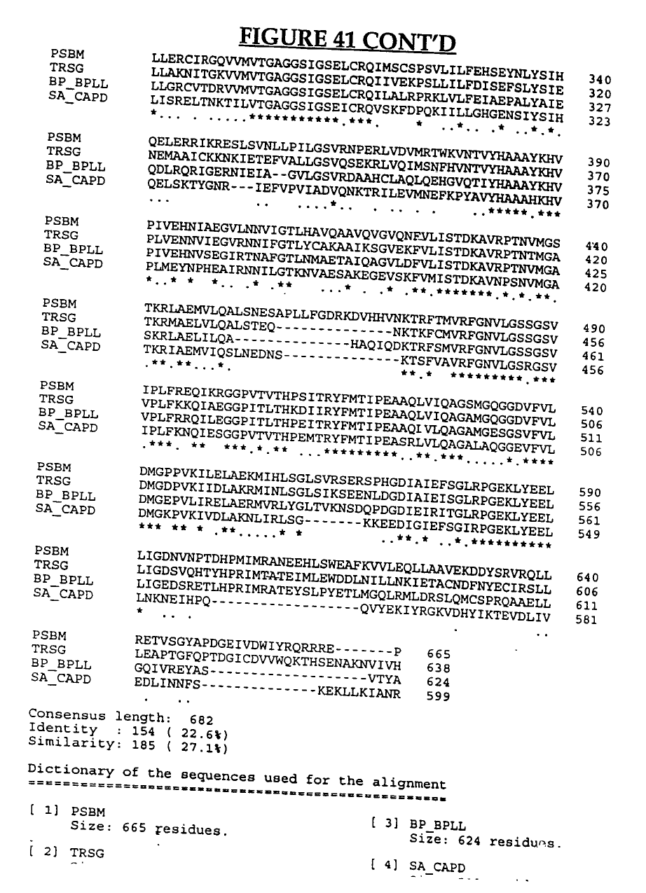

The psbM gene follows the IS⁴⁰⁷_{Pa} element and may be transcribed from one of three potential promoters present in the right IR (FIG. 31). A gene-activating promoter was previously shown to be present in the right IR of IS⁴⁰⁷_{Bc} (Wood et al., 1991). psbM is unusual because in contrast to other psb genes described above, it hybridizes to chromosomal DNA from all 20 serotypes (Table 1). PsbM mutants, generated by insertion of a gentamicin cassette into a unique NruI site within psbM, exhibit B-band LPS-minus phenotype. This confirms the involvement of the psbM product in LPS biosynthesis, despite the fact it lies outside of the O5-specific region (FIG. 41). PsbM has homology to a range of proteins involved in exopolysaccharide synthesis, including BplL from the B. pertussis LPS cluster (Allen and Maskell, 1996), TrsG from the core biosynthetic cluster of Y. enterocolitica O3 (Skurnik et al., 1995), and CapD from the S. aureus capsular gene cluster (Lin et al., 1994). These homologies are summarized in Table 2.

As shown previously for BplL, only the carboxy half of the PsbM protein has homology to GalE from several bacterial species, suggesting it may have originated as a fusion protein. In support of this hypothesis, PsbM also has homology to two adjacent ORFs (ORF10 and ORF11) in the LPS cluster of V. cholerae O139 (Comstock et al., 1996). The homology to ORF10 and ORF11 lies in the amino-terminal and carboxy-terminal half of PsbM, respectively (Table 2), suggesting that two similar ORFs were fused during the evolution of PsbM and the BplL/TrsG/CapD group.

Based on these homologies, PsbM is thought to be involved in the biosynthesis of the N-acetylfucosamine residue of the O5 O-antigen. As mentioned above, the O-antigen of B. pertussis and the type 1 capsule of S. aureus and the outer core of Y. enterocolitica O3 all contain N-acetylfucosamine. PsbM could function as a dehydrogenase, and it contains two putative NAD-binding domains (FIG. 33), as do BplL and TrsG. Again, these duplications may have arisen from an ancestral fusion of two NAD-binding domain-containing proteins and may be bifunctional.

psbN.

The psbN gene has some homology to eryA, a gene involved in erythromycin biosynthesis in Sacchropolyspora erythrae. Generation of knockout mutations in psbN will demonstrate its function in biosynthesis of the O5 O-antigen.

uvrB.

The last partial open reading frame present on pFV100 has high homology to the highly conserved uvrB gene from several bacterial species, including E. coli, S. enterica sv Typhimurium, and Micrococcus luteus. UvrB is a subunit of the UvrABC DNA excision repair complex involved in removal of thymidine dimers induced by irradiation with ultraviolet light. The presence of uvrB adjacent to psbN confirms that psbN is the last gene in the psb cluster that could be involved in O-antigen biosynthesis.

Organization of the psb Gene Cluster in P. aeruginosa O5.

Several entire rfb clusters, particularly from enteric bacteria, have been characterized to date (reviewed in Whitfield and Valvano, 1993; and Schnaitman and Klena, 1993). In general, rfb clusters are located on the chromosome adjacent to the his operon and the gnd gene. Amongst the enterics, it has previously been shown that the rfb clusters are organized in a specific fashion (Reeves, 1993; Schnaitman and Klena, 1993). Genes necessary for sugar biosynthesis are arranged in discrete blocks located 5′ to the transferases and other assembly genes (rfbX, rfc and rol). The psb cluster, however, appears to be almost randomly organised, with genes thought to be involved in the biosynthesis of Man(2NAc3N)A and Man(2NAc3NAc)A scattered throughout the gene cluster (psbI, psbE, psbD, psbB and psbC). The genes thought to encode for the biosynthesis of FucNAc are also scattered throughout the cluster (psbK, psbM, psbG, psbN). Further, the genes encoding transferases are interspersed throughout the psb cluster (psbH, psbJ, psbL), and are separated from one another by one gene each. However, the transferase genes do appear to be organized such that the gene encoding the putative first transferase (PsbL), thought to initiate O-antigen assembly on undecaprenol, is the most distal. Recent results from detailed spectroscopic analysis, using high resolution NMR and Mass Spectroscopy of an rfc mutant of PAO1, strain AK1401, show that FucNAc is the first sugar of the O-antigen unit, attached to the core oligosaccharide. PsbL's homology to Rfe, and its hydropathicity support the interpretation that it is the first transferase, and is responsible for attachment of the FucNAc residue to undecaprenol. Therefore, based on their gene order and their relative hydropathic indices (−0.21 and 0.10), the psbJ and psbH gene products are thought to transfer Man(NAc)₂A and Man(2NAc3N)A, respectively.

The O-antigen of P. aeruginosa O5 is an Rfc-dependent Heteropolymer.

The psb cluster was shown to contain an rfc gene, (See Example 1) the interruption of which (by knockout mutation and gene replacement) resulted in a SR phenotype (de Kievit et al., 1995). At least two other gene products, Rol and RfbX, are thought to be involved in Rfc-dependent synthesis of heteropolymeric O-antigens (Whitfield, 1994). Here a rol gene has been identified in the psb cluster. However, in the analysis of the psb genes, no rfbX-like gene was identified. The psbF gene product appeared to be the most likely candidate, based on its hydropathy profile (FIG. 9), but insertional mutants of psbF do not have the phenotype expected of rfbX mutants.

Identification of his Genes within the psb Gene Cluster.

The identification of the hisH and hisF genes in the middle of the psb cluster raises some interesting evolutionary questions. It appears that these two his genes are not native to P. aeruginosa, because they have a lower %G+C content than background (50% vs.67%) and they hybridize only to a limited number of serotypes with related O-antigens instead of all 20 serotypes. It is not uncommon for his operons to be located adjacent to rfb clusters, and it is likely that the his genes were acquired simultaneously with some or all of the psb genes. The lack of significant homology with any of the HisF and HisH proteins characterized to date, and particularly with those of other Gram-negative bacteria precludes the use of these genes as evolutionary “luggage tags”. The lack of homology with other Gram-negative HisH/F proteins suggests either they came from an as-yet uncharacterized source or that they have been resident in P. aeruginosa for a long time. The latter possibility is bolstered by the divergence over time of the O-antigen structures/genes from the ancestral psb cluster in the five O5-related serotypes in which these hisH and hisf genes are found.

The location of hisH and hisF adjacent to one another is unique in bacteria. The similarity between hisH and hisA genes, and the usual location of hisA, rather than hisH, adjacent to hisF, raises the possibility that the P. aeruginosa psb hisH gene was originally a hisA gene that has diverged so as to be more similar to hisH than to hisA. However, there is precedent for the juxtaposition of hisH and hisF; in the yeast Sacchromyces cerevisiae, the homologues of the hisH and hisF genes are adjacent, and are fused into one translational unit called HIS7 (Kuenzler et al., 1993). Alternatively, the hisHF arrangement may be ancestral to the duplication event which resulted in the hisHAF gene order. Another possibility is that the hisA gene may have been lost, leaving hisH and hisf adjacent.

psb Gene Dissemination Amongst the 20 Serotypes of P. aeruginosa.

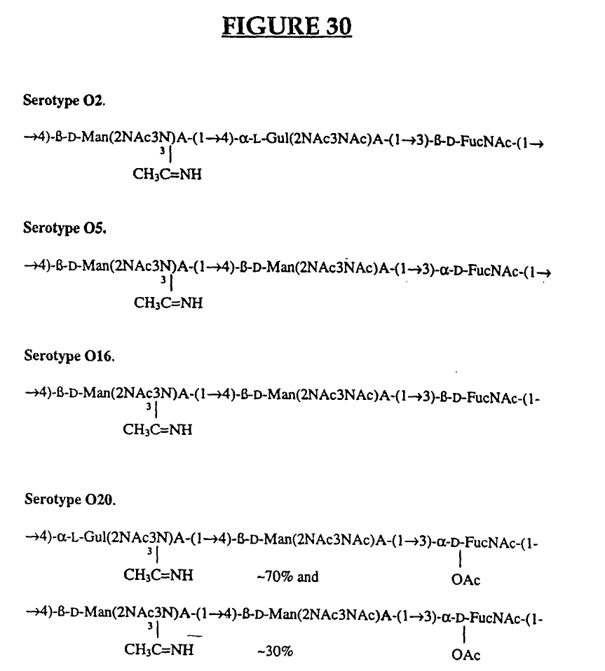

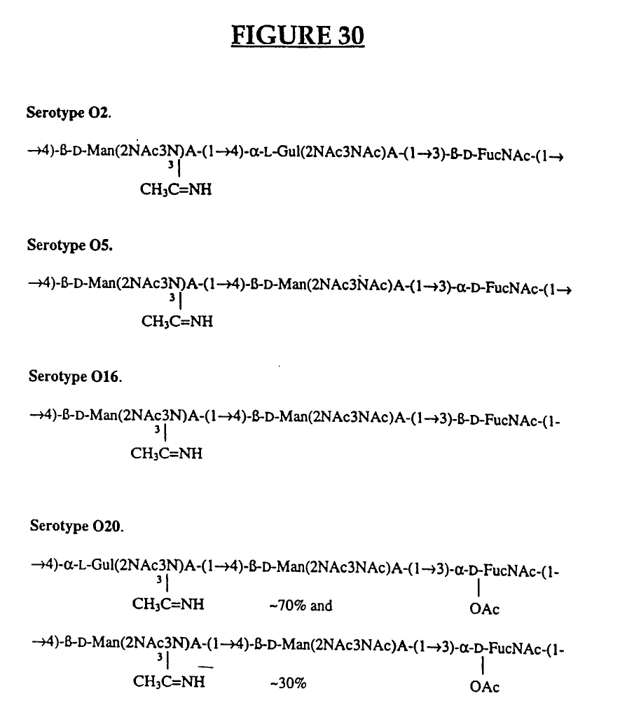

The observation that no genes were found in the O5 cluster which hybridize only to chromosomal DNA from serotype O5 and not to the other related serotypes was intriguing. The differences among these five serotypes is confined to changes in the type of linkage between sugars or to the epimer present in the O-antigen, either mannuronic or guluronic acid (FIG. 30). These differences could result from variation in transferase activity or in epimerization activity, respectively. Further analysis of the putative transferase activities will be necessary to determine whether there are differences in activity among serotypes despite the obvious homology at the genetic level. It will be interesting to determine whether the introduction of multicopy plasmids containing the O5 transferase genes into the related serotypes will result in an alteration in O-antigen structure that could be detectable with serotype-specific monoclonal antibodies. There is precedence for this, as a P. aeruginosa strain PAO1 (serotype O5) phage induced mutant, strain AK1380, was isolated which was identified as serotype O16 (see Lam et al., 1992, FIG. 30; and Kuzio and Kropinski, 1993).

The genetic differences among the five serotypes with related O-antigens are obviously quite minor. Comparison of the DNA sequences of the O2 rfc and the O5 rfc genes revealed they are very homologous at the nucleotide level).

### Example 4

Further Characterization of Rol (Wzz) Gene and Region Upstream

In this example the rol gene is generally referred to as the wzz gene.

The materials and methods used in Example 4 are as follows:

Bacterial Strains and Plasmids.

The bacterial strains and plasmids used in this study are listed in Table 8. P. aeruginosa strains were cultured either on Luria broth or plates or on Pseudomonas Isolation Agar (PIA: Difco, Detroit, Mich.). E. coli strains were cultured on Luria broth or plates. Media were supplemented with antibiotics ampicillin, carbenicillin, tetracycline, or gentamicin (all from Sigma, St. Louis, Mo.) as required, using the concentrations outlined in de Kievit et al., 1995.

DNA Methods.

Chromosomal DNA was isolated from P. aeruginosa using the method of Goldberg and Ohman, 1984. Plasmid and cosmid DNA was isolated using the Qiagen midi-prep kit (Qiagen Inc., Chatsworth, Calif.) as directed by the manufacturer. Restriction and modification enzymes were supplied by Gibco/BRL (Gaithersburg, Md.), Boehringer Mannheim (Laval, PQ), and/or New England Biolabs (Beverly, Mass.) and were used as directed by the manufacturers.

Plasmids were introduced into E. coli by CaCl₂ transformation (Huff et al., 1990) and into P. aeruginosa by electroporation using a BioRad (Richmond, Calif.) Gene Pulser apparatus following manufacturers protocols. P. aeruginosa electrocompetent cells were prepared by washing early log phase cells twice for 5 min each in sterile 15% room-temperature glycerol followed by immediate resuspension in the same solution. Cells were either used immediately or frozen at −80° C. for future use. Alternatively, plasmids were mobilized into P. aeruginosa through biparental mating with E. coli SM10 carrying plasmids of interest (Simon et al., 1983).

Construction of Plasmids.

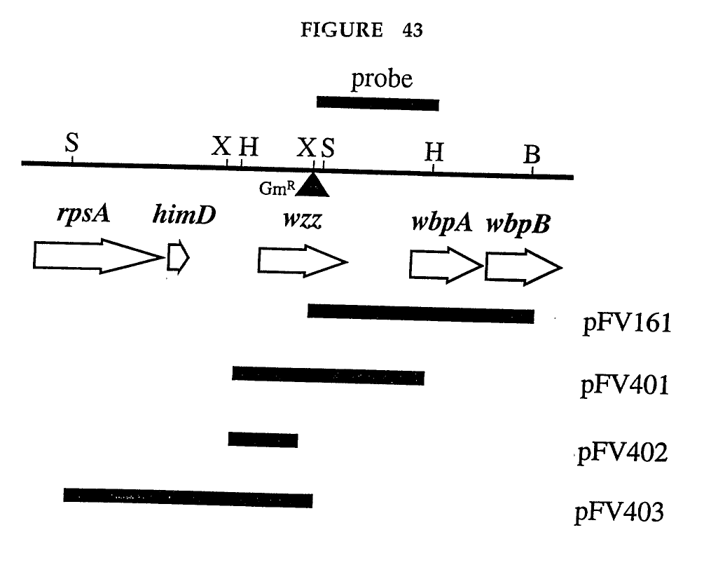

The cosmid pFV100, containing the P. aeruginosa wbp cluster, was used as a source of DNA for the construction of pFV161 (FIG. 43). An overlapping cosmid, pFV400, was the source of a 2.3-kb HindIII fragment cloned into pBluescript II SK (pFV401). For DNA sequencing, a 0.8 kb HindIII-XhoI fragment from pFV401 was subcloned into pBluescript II SK (pFV402). A 3.0 kb SstI fragment containing the 5 portion of wzz and upstream sequences was cloned from pFV400 into pBluescript II SK (pFV403). For complementation experiments, the 2.3 kb insert of pFV401 was cloned into the Pseudomonas-E. coli shuttle vector pUCP26 (Table 14), downstream of the vectors lacZ promoter (pFV401-26).

DNA Sequencing and Analysis.

Using the above plasmids, the DNA sequences of both strands of the pFV401 insert were determined by the GenAlyTiC facility (University of Guelph, Guelph, ON) employing the Taq DyeDeoxy Terminator Cycle Sequencing Kit (Applied Biosystems, Mississauga, ON) and an Ericomp Model TCX15 Thermal cycler. Oligonucleotide primers were synthesized on an Applied Biosystems model 391 DNA synthesizer and purified as directed by the manufacturer.

DNA sequences were collated and analyzed using GENE RUNNER for Windows (Hastings Software, Newark, N.J.), DNAsis for Windows (Hitachi Software, Helixx, Scarborough, ON), and PC/GENE (IntelliGenetics Inc., Mountain View, Calif.). DNA and protein database searches were performed using the NCBI BLAST network server (Altschul et al., 1990; Gish and States, 1993).

Expression of the Wzz Protein.

An E. coli S30 extract in vitro protein expression kit (Promega, Madison, Wis.) was used to examine the product encoded by the O5 wzz gene. Column-purified (Qiagen) plasmid DNA of pBluescript II SK, pFV401a (containing the O5 wzz gene cloned downstream of the lacZ promoter of pBluescript II SK) and pFV401b (containing the same DNA cloned in the opposite orientation) were used as templates in the coupled transcription/translation reaction in the presence of ³⁵S-labelled methionine (Trans35-Label, ICN, Costa Mesa, Calif.). The labelled proteins were precipitated with acetone, separated on standard discontinuous 12.5% SDS-PAGE along with unstained BioRad low-molecular-weight markers and visualized by autoradiography using ³⁵S-sensitive film (BioMax, Kodak, Toronto, ON).

Preparation and Visualization of LPS.

LPS from P. aeruginosa was prepared by the method of Hitchcock and Brown, 1983. The LPS preparations were separated on standard discontinuous 12.5% SDS-PAGE gels and visualized by silver staining using the method of Dubray and Bezard, 1982. Alternatively, LPS separated on SDS-PAGE gels was transferred to nitrocellulose and visualized by immunoblotting (Burnete, 1981). Nitrocellulose blots were blocked with 3% skim milk followed by overnight incubation with hybridoma culture supernatants containing MAb MF15-4 (specific for O5 B-band LPS), MAb 18-19 (cross-reactive for O2, O5, and O16 B-band LPS core-plus-one O-antigen unit; 28) or MAb N1F10 (specific for A-band LPS; 30). The second antibody was a goat anti-mouse F(ab)₂-alkaline phosphatase conjugate (Jackson Laboratories, Bio/Can Scientific, Mississauga, ON). The blots were developed using a substrate containing 0.3 mg/ml NBT (Nitro Blue Tetrazolium) and 0.15 mg/ml BCIP (5-bromo-4-chloro-3-indolyl phosphate toluidine) (Sigma) in 0.1 M bicarbonate buffer (pH 9.8).

Creation of Wzz Knockout Mutants Through Gene Replacement.

The gene replacement strategy of Schweitzer and Hoang, 1985 was used for generation of knockout mutations in wzz. The 2.3 kb HindIII insert of pFV401 was cloned into pEX100T, a pUC19-based vector containing the sacB gene as a selectable marker (pFV401T). An 875 bp gentamicin resistance cassette from the plasmid pUCGM was then cloned into the unique XhoI site within the insert (pFV401TGm). Constructs containing the interrupted wzz gene were mobilized into P. aeruginosa O5 by biparental mating with E. coli SM10. Since pEX100T does not replicate in P. aeruginosa, selection for gentamicin resistance allows detection of chromosomally-integrated copies of the mutated gene. Determination of sucrose and carbenicillin (Cb) sensitivities distinguishes between merodiploids (sucroses, CbR) and true recombinants (sucrose^{R}, Cb^{S}). The presence of the gentamicin cassette in the chromosomal DNA of P. aeruginosa O5 and O16 wzz mutants was confirmed by Southern blot analysis (not shown).

Results

Cloning and Sequencing of the P. aeruginosa O5 Wzz Gene.

Nucleotide sequences with homology to wzz from E. coli, Salmonella enterica sv Typhimurium and Shigella flexneri (Bastin et 1993; Batchelor et al., 1992; Morona et al., 1995) were identified ending approximately 800 bp upstream of the first gene of the P. aeruginosa O5 wbp gene cluster, wbpA (FIG. 43). The amount of DNA with homology to wzz was 479 bp, starting at the XhoI cloning site of the insert of pFV100 and ending with a stop codon. Based on the average size (1 kb) of previously characterized wzz genes (Bastin et al., 1993; Batchelor et al., 1992; Morona et al., 1995), this sequence represented approximately half of the putative P. aeruginosa wzz gene.

A 1.5 kb XhoI-HindIII fragment from pFV161 containing the 3 end of the putative wzz gene (FIG. 43) was used as a probe to screen a P. aeruginosa O5 cosmid library. One cosmid (pFV400) which hybridized with the probe was isolated. A probe-reactive 2.3 kb HindIII fragment from pFV400 was subcloned into pBluescript II SK to form pFV401 (FIG. 43).

DNA sequence analysis revealed an open reading frame (ORF) of 1046 base pairs (bp), sufficient to encode a protein of 348 amino acids with a molecular mass of 39.3 kilodaltons (kDa), and an isoelectric point of 6.26. Comparison of the deduced amino acid sequence of the P. aeruginosa O5 protein with those in GenBank revealed from 11.5 to 20.0% amino acid identity with Wzz-like proteins of other species (Table 15). P. aeruginosa Wzz also has similarity with proteins thought to be involved in polymerization or export of exopolysaccharide capsules in E. coli O8/O9 (13, 15; accession #U39306), Vibrio cholerae O139 (4; OtnB, X90547), Klebsiella pneumoniae (ORF6, 747665), and Rhizobium meliloti (ExoP, Z22636). P. aeruginosa Wzz also has similarity with FepE from E. coli, thought to be a component of the ferric enterobactin permease (Ozenburger et al., 1987; X74129).

While there is poor primary sequence homology between the Wzz protein of P. aeruginosa O5 and related proteins, their predicted secondary structures are similar (FIG. 44). There are conserved hydrophobic regions at both the amino and carboxy termini, and hydrophilic regions in the central portion of the protein. The predicted transmembrane helices in P. aeruginosa O5 Wzz are between amino acids 29-49 and 319-339. These hydrophobic regions contain the amino acid residues which are most highly conserved among Wzz-like proteins.

Analysis of the Region Upstream of Wzz.

The wzz gene is upstream of the wbp cluster of P. aeruginosa O5. As described in Example 3, most of the genes in this cluster, including wzz, are serogroup-specific, and are found only in serotypes O2, O5, O16, O18, and O20. These serotypes have chemically- and structurally-related O antigens (Knirel and Koch et Kov., 1994). Based on Southern blot hybridization results, the 5 end of the serogroup-specific region was previously localized to a 1.9-kb SstI-XhoI fragment located 1.1 kb upstream of the 5 end of pFV100. DNA sequence analysis of this fragment revealed a gene with 85% nucleotide identity with the E. coli gene rpsA, encoding 30S ribosomal protein S1 (Schnier et al., 1982), and a second gene which has 98% identity with P. aeruginosa himD, encoding the β subunit of integration host factor (IHF) (Delic-Atree et al., 1995). The rpsa and himD genes are transcribed in the same direction as wzz. These data locate rpsA and himD adjacent to the wbp cluster at 37 minutes on the chromosomal map of P. aeruginosa O5 strain PAO1 (Holloway et al., 1994; Lightfoot and Lam, 1993).

Expression of the Putative Wzz Protein.

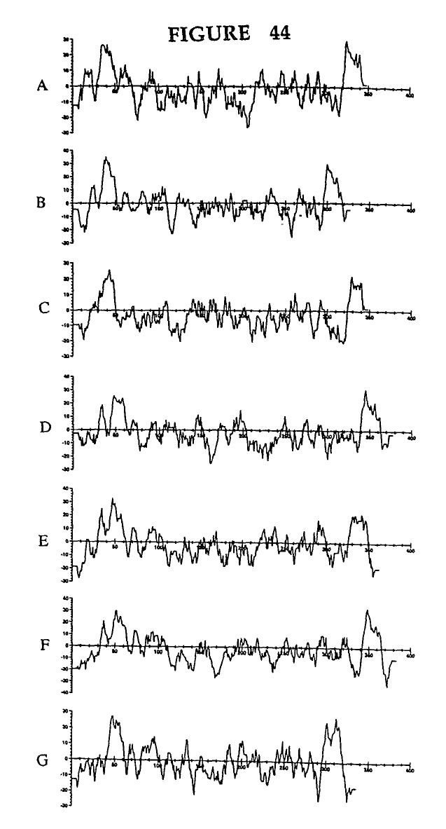

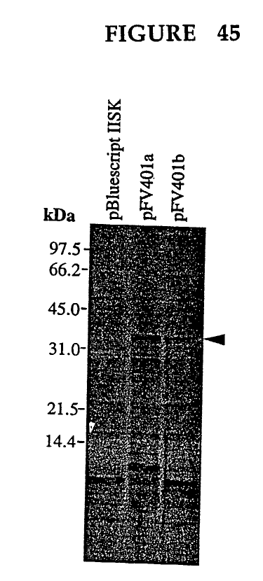

Using an E. coli S30 extract expression system, the putative wzz gene was shown to encode a protein with an apparent molecular weight of 40 kDa which was not present in samples containing only the vector, pBluescript II SK (FIG. 45). The estimated size of 40 kDa is in good agreement with that predicted from the DNA sequence (39.3 kDa). A reduced amount of the same protein was detected in the sample in which the insert DNA was cloned in the opposite orientation (pFV401b), indicating that there is a native promoter present upstream of the wzz gene which functions weakly in E. coli. Examination of the DNA sequence upstream of wzz revealed at least three potential promoter sequences with partial homology to the E. coli δ⁷⁰ consensus. The −10 regions of these putative promoters are located approximately 60, 140, or 155 bp upstream of the wzz initiation codon.

Analysis of the Putative Wzz Protein Function Using Chromosomal Knockout Mutants.

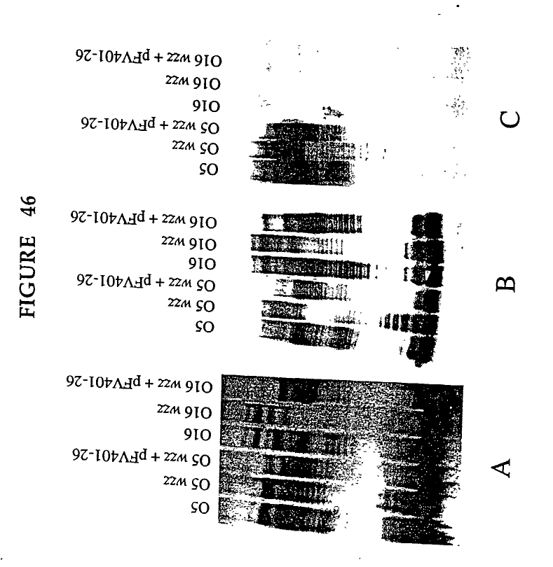

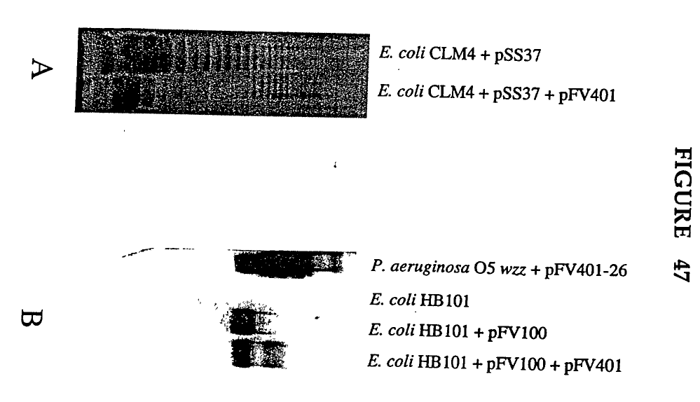

A gentamicin-resistance (Gm^{R} ) cassette was inserted into the putative wzz gene of P. aeruginosa O5, and the interrupted gene was reintroduced into the O5 chromosome by homologous recombination. Comparison of LPS from the wild-type strain and the Gm^{R} mutant on silver-stained SDS-PAGE gels and Western immunoblots using B-band-specific MAbs MF15-4 and 18-19 showed that the mutant had an altered LPS banding pattern. When MAb 18-19 was used, the LPS from the wzz mutant showed an increase in both shorter and longer B-band LPS O chains and a decrease in B-band O chains whose length corresponded to that preferred in the O5 parent strain (FIG. 46). On the immunoblot using MAb MF15-4, which is specific for high-molecular-weight LPS (Lam et al., 1992), there is also an increase in both shorter and longer B-band O chains. Similar Western immunoblots using the A-band LPS-specific MAb N1F10 showed the modality of A-band was unaffected by the wzz mutation (not shown). Although the B-band LPS pattern of the wzz mutant is significantly different from the parent strain, it does not show the linear distribution of O-antigen chain lengths seen in enteric wzz mutants (FIG. 47A). Reintroduction of the O5 wzz gene on pFV401-26 restored the mutant to a phenotype similar to that of the parent but missing both the shortest and longest groups of chain lengths (FIG. 46).

Comparison of the Function of Wzz in Two Related Serotypes of P. aeruginosa.

A DNA probe containing the O5 wzz gene hybridized with chromosomal DNA only from serotypes O2, O5, O16, O18, and O20 of P. aeruginosa, all of which have chemically- and structurally-related O antigens (Example 3). The O antigens of both O5 and O16 are composed of two mannuronic acid and one N-acetyl fucosamine residues, but differ in one glycosidic linkage. In O5, the linkage is (1(3)-(-D-Fuc2NAc, while in O16, the linkage is (1(3)-(-D-Fuc2NAc. This change results in a discernible difference in the LPS patterns of O5 and O16 (FIG. 46).

Taking advantage of the similarity between the O-antigen gene clusters of O5 and O16, a wzz knockout mutation was introduced into O16, using the O5 wzz knockout construct. As an additional benefit, O16 does not express A-band LPS (Lam et al., 1989), thus any changes in B-band LPS patterns on silver-stained gels were more easily visualized. The structural difference between O5 and O16 LPS is detected by MAb MF15-4, which recognizes only O5 and not O16 LPS. To examine LPS from both O5 and O16 simultaneously on Western immunoblots, MAb 18-19, which cross-reacts with all five serotypes in the O5 serogroup (Lam et al., 1992), was used. Comparison of LPS from the wild-type O16 parent and the O16 wzz knockout mutant showed the mutant displayed a loss of modality corresponding to the preferred chain lengths of the parent, and an increase in higher-molecular-weight LPS (FIG. 46). Interestingly, there still appeared to be chain length modulation in the O16 wzz mutant that was different from that of the parent, with a decrease in short O chains in comparison to the O5 wzz mutant. Bastin and coworkers (1996) showed that the modality of chain length distribution was dependent on the source of the zvzz gene. However, the pattern of LPS chain length distribution of O16 wzz mutants carrying the O5 wzz gene on pFV401-26 resembled that of the O16 parent strain, rather than the O5 strain (FIG. 46).

Ability of the P. aeruginosa O5 wzz gene to function in E. coli.

In order to determine whether wzz from P. aeruginosa O5 could complement an enteric wzz mutation, E. coli strain CLM4, which is deleted for O-antigen genes including wzz (Marolda and Valvano, 1993), was used. CLM4 was transformed with either pSS37 (containing the O-antigen biosynthetic genes from S. dysenteriae type I without a wzz gene alone, or with both pSS37 and pFV401, containing P. aeruginosa O5 wzz. While LPS from E. coli CLM4/pSS37 showed an unregulated distribution of chain lengths, LPS from E. coli CLM4/pSS37/pFV401 showed a restoration to modality, with a decrease in short and very long O chains, and an increase in chains with approximately 10-20 repeats (FIG. 47A).

The core oligosaccharide of the E. coli K-12 hybrid strain HB101, but not K-12 itself, can act as an acceptor for P. aeruginosa O antigens (Goldberg et al., 1992; Lightfoot and Lam, 1993). The structure of the HB101 core has not been elucidated. Although E. coli HB101 carrying pFV100 had previously been shown to express LPS which could be recognized by B-band-specific MAb MF15-4, its chain-length regulation had not been examined. pFV100 is now known to contain a truncated wzz gene. The expression of LPS from E. coli HB101 carrying both pFV100 and the complete O5 wzz gene on pFV401 was examined. E. coli HB101 carrying pFV100 alone expressed an O5 O antigen with modulated, short-chain O-antigen molecules (FIG. 47B). When both pFV100 and pFV401 were present in E. coli HB101, a dual LPS banding pattern was visible on Western immunoblots (FIG. 47B). The coexpression of both E. coli and P. aeruginosa Wzz proteins resulted in a major group of short O chains attributable to HB101 Wzz, and a minor group with longer chains attributable to the P. aeruginosa O5 Wzz protein.

The identification of the rpsA and himD genes upstream of wzz completes the delineation of the region of serogroup-specific DNA responsible for encoding the B-band LPS 0 antigen of P. aeruginosa O5 and related serotypes. The entire O5 wbp cluster is thus bounded by himD on the 5 end and uvrB on the 3 end and is approximately 24.3 kb from the start of wzz to the end of wbpN. The serogroup-specific portion is approximately 18.4 kb from the start of wzz to the end of wbpL. Unlike enteric O-antigen (rfb) clusters, the wbp cluster is not flanked by his and gnd, although there are two his genes, hisH and hisF, located in the center of the cluster. The location of wzz upstream of the wbp cluster in P. aeruginosa is opposite to that in many enteric bacteria, where wzz is located downstream of the O-antigen cluster (Batchelor et al., 1992; Morona et al., 1995). The presence of the rpsA and himD genes, which are highly conserved among bacterial species, at the junction between the serogroup-specific and common regions suggests they may have been the site of a past recombination event. himD encodes the β-subunit of IHF which has previously been shown to be involved in regulation of biosynthesis of the exopolysaccharide alginate (Wozniak and Ohman, 1993; Wozniak, 1994).

The presence of a functional wzz gene in P. aeruginosa O5 confirms that both the O-antigen polymerase, Wzy, and Wzz are required for expression of the heteropolymeric B-band O antigen, as predicted by current models. Growing evidence suggests that Wzz proteins may also play a role in the modulation of the length of capsular exopolysaccharide polymers (Bik et al., 1996; Dodgson et al., 1996; Franco et al., 1996). A possible homologue of the third component of Wzy-dependent systems, Wzx, is present in the wbp cluster (Burrows et al., 1996).

The LPS banding pattern of enteric wzz mutants consists mainly of short O chains with steadily decreasing amounts of longer chains (FIG. 47A). In contrast, neither the O5 nor the O16 wzz mutants display this typical wzz phenotype, and the O16 mutant in particular continues to display some chain length regulation. It is possible that chain length regulation in P. aeruginosa is not simply dependent on wzz. In the case of O16, there may be a second wzz gene present in the O16 chromosome whose activity is normally masked by the wzz of the O5 serogroup. Complementation of the O5 and O16 mutants by wzz on a multicopy plasmid gave rise to strains whose LPS appeared even more tightly regulated for size than that of the parent strains, since the complemented wzz mutants lacked both short- and very long-chain modal groups, and had an increase in medium-length groups. One possible interpretation of these results is that the regulation of chain length by wzz in P. aeruginosa is normally imprecise, giving rise to groups with multiples of the preferred chain length instead of a single group. This interpretation fits the model of Bastin et al., 1993 who suggested that multimodal distributions of chain lengths could result from reinitiation of polymerization without an intervening ligation step.

Complementation of the O16 mutants by the O5 wzz gene restored them to a phenotype resembling the O16 parent. Contrary to the findings of Bastin and colleagues, 1993, these results show that in these closely-related serotypes, the structure of the O antigen, or possibly difference in the O5 vs O16 genetic background, determines the preferred O-antigen chain length. While the O16 wzz and wzy genes have not been isolated, they are probably highly similar to those of O5 based on the results of high-stringency Southern blot analysis. The analysis of wzy from the related serotypes O2 and O5 demonstrated that the genes are essentially identical.

The P. aeruginosa O5 Wzz protein can modulate expression of both homologous (P. aeruginosa O5) and heterologous (S. dysenteriae) O antigens in E. coli although it has only 20% identity with the Wzz protein of E. coli. The ablility of P. aeruginosa Wzz to modulate a heterologous O antigen is consistent with previous work showing Wzz is not specific for O-antigen type. When E. coli and P. aeruginosa Wzz proteins are coexpressed in E. coli, the modulating effect of the native protein predominates although the P. aeruginosa wzz is present in multicopy. This difference can be seen in the increased proportion of short O chains versus longer O chains which are expressed. Despite variations in efficacy, it appears that the Wzz proteins from different Gram-negative families function in an analogous manner and can act as interchangeable components of the O-antigen assembly complex.

The ability of Wzz, Wzy and WaaL proteins with divergent primary sequences to act reciprocally suggests that they are interacting through recognition of common, conserved structural features. Although the amino acid similarities between the Wzz proteins are low, their secondary structures are alike (FIG. 44). Similarly, although the primary sequence similarities of the Wzy proteins from a number of bacteria are poor, all have highly similar secondary structures containing multiple membrane-spanning domains (Cryz et al., 1984). Comparison of the WaaL proteins from E. coli and S. enterica sv Typhimurium, the only O-antigen ligases characterized to date, show that they too have conserved secondary structures, but less than 20% primary sequence homology (Liu and Wang, 1990). In light of this information, it is now possible to target conserved structural features of these proteins for modification in order to further define the areas critical for putative protein interactions.

Having illustrated and described the principles of the invention in a preferred embodiment, it should be appreciated to those skilled in the art that the invention can be modified in arrangement and detail without departure from such principles. We claim all modifications coming within the scope of the following claims.

All publications, patents and patent applications referred to herein are incorporated by reference in their entirety to the same extent as if each individual publication, patent or patent application was specifically and individually indicated to be incorporated by reference in its entirety.

Below-full citations are set out for the references referred to in the specification and detailed legends for the figures are provided.

The application contains sequence listings which form part of the application.

TABLE 1
Pseudomonas aeruginosa serotype O5 wbp gene cluster.

| locus | base positions | % G + C | MW encoded | AAs^{d} | pI^{e} | H.I.^{f} | distribution^{g} |
|---|---|---|---|---|---|---|---|
| wZZ^{a} | 1-479 | 49.5 | 38.6 kDa | 158 | nd | nd | 2, 5, 16, 18, 20 |
| wbpA | 1286-2596 | 54.5 | 48.2 kDa | 436 | 5.36 | −0.08 | 2, 5, 16, 18, 20 |
| wbpB | 2670-3620 | 52.8 | 35.8 kDa | 316 | 6.40 | −0.27 | 2, 5, 16, 18, 20 |
| wbpC | 3689-5578 | 53.1 | 69.9 kDa | 629 | 9.06 | 0.48 | 2, 5, 16, 18, 20 |
| wbpD | 5575-6066 | 53.9 | 17.4 kDa | 163 | 8.25 | 0.19 | 2, 5, 16, 18, 20 |
| wbpE | 6152-6982 | 52.8 | 29.9 kDa | 276 | 5.26 | −0.01 | 2, 5, 16, 18, 20 |
| wzy^{b} | 7236-8552 | 44.6 | 48.9 kDa | 438 | 9.63 | 0.80 | 2, 5, 16, 18, 20 |
| wbpF | 8549-9499 | 49.0 | 33.8 kDa | 316 | 9.49 | 0.99 | 2, 5, 16, 18, 20 |
| hisH | 9831-10388 | 49.3 | 20.9 kDa | 185 | nd | nd | 2, 5, 16, 18, 20 |
| hisF | 10388-11143 | 50.0 | 27.5 kDa | 251 | nd | nd | 2, 5, 16, 18, 20 |
| wbpG | 11281-12411 | 44.5 | 43.4 kDa | 376 | 8.15 | −0.38 | 2, 5, 16, 18, 20 |
| wbpH | 12427-13548 | 45.6 | 42.0 kDa | 373 | 8.79 | −0.21 | 2, 5, 16, 18, 20 |
| wbpI | 13545-14633 | 50.2 | 39.7 kDa | 362 | 5.40 | 0.06 | 2, 5, 16, 18, 20 |
| wbpJ | 14651-15892 | 54.5 | 45.3 kDa | 413 | 6.54 | 0.10 | 2, 5, 16, 18, 20 |
| wbpK | 15889-16851 | 56.8 | 34.4 kDa | 320 | 9.03 | 0.14 | 2, 5, 16, 18, 20 |
| wbpL^{c} | 16911-17822 | 55.5 | 32.9 kDa | 303 | 9.08 | 0.84 | 2, 5, 16, 18, 20 |
| IS1209 | 17935-19144 | 59.3 | nd | n/a | n/a | n/a | 1 to 11, 13 to 20 |
| wbpM | 19678-21675 | 61.9 | 74.5 kDa | 665 | 9.33 | 0.09 | 1 to 20 |
| wbpN | 22302-23693 | 63.6 | 48.5 kDa | 463 | 6.12 | −0.09 | 1 to 20 |
| uvrB^{a} | 23704-24417 | 61.2 | 26.7 kDa | 238 | nd | nd | 1 to 20 |

^{a}truncated ORF
^{b}de Kievit et al. (1995)
^{c}wbpL was originally named rfbA; Dasgupta and Lam (1995)
^{d}number of amino acids
^{e}isoelectric point of the protein, calculated using GeneRunner for Windows (Hastings Software).
^{f}hydropathic index of the protein, calculated using DNAsis for Windows (Hitachi Software). Positive values indicate the protein is hydrophobic, while negative values indicate the protein is hydrophilic.
^{g}distribution of this gene among the 20 serotypes of P aeruginosa, based on positive hybridization in high-stringency Southern blot analysis.

TABLE 2
Similarities of P. aeruginosa O5 Wbp proteins to those in the databases.

| | | | | Database |
|---|---|---|---|---|
| P. aeruginosa | | | % identity | accession |
| protein | Similar proteins | Putative function | (% similarity)* | number |
| WbpA | EpsD-Burkholderia solanacearum | dehydrogenase | 33.1 (50.6) | U17898 |
| | CapL-Staphylococcus aureus | capsule synthesis | 31.6 (45.3) | U10927 |
| | VipA-Salmonella enterica sv Typhi | Vi antigen synthesis | 30.8 (44.9) | D14156 |
| | RffD (o379)-Escherichia coli | UDP-ManNAc dehydrogenase | 30.2 (42.8) | M87049 |
| WbpB | LmbZ-Streptomyces lincolnesis | oxidoreductase | 19.3 (28.2) | X79146 |
| | BplA-Bordetella pertussis | dehydrogenase | 12.4 (17.0) | X90711 |
| | Pur10-Str. alboniger | oxidoreductase | 5.7 (12.0) | X92429 |
| WbpC | HI0392-Haemophilus influenzae | unknown | 24.9 (37.2) | U00073 |
| | ExoZ-Rhizobium meliloti | O-acylase | 27.4 (40.3) | U50300 |
| | AcyA-Str. thermotolerans | O-acylase | 24.9 (37.2) | X58126 |
| | unknown-Caenorhabditis elegans | unknown | 18.0 (26.7) | D30759 |
| | NodX-R. leguminosarum | O-acylase | 16.3 (23.1) | X07990 |
| WbpD | BplB-B. pertussis | acetylase | 73.6 (83.4) | X90711 |
| | CysE-Buchnera aphidicola | serine O-acetylase | 28.2 (45.4) | M90644 |
| | CysE-Arabidopsis thalnia | serine O-acetylase | 30.7 (42.4) | L42212 |
| | CysE-H. influenzae | serine O-acetylase | 28.2 (39.9) | U32689 |
| | CysE-E. coli | serine O-acetylase | 28.8 (38.6) | M15745 |
| WbpE | BplC-B. pertussis | aminase | 64.1 (75.7) | X90711 |
| | DegT-Bacillus subtilis | dideoxy sugar biosynthesis | 51.2 (62.4) | M29002 |
| | ERYC1-Saccharopolyspora erythrae | dideoxy sugar biosynthesis | 37.3 (48.2) | P14290 |
| | SpsC-Ba. subtilis | dideoxy sugar biosynthesis | 37.4 (53.3) | P39623 |
| | Dnrj-Str. peucetius | dideoxy sugar biosynthesis | 34.1 (50.4) | P25048 |
| WbpF | ExoT-R. meliloti | succinoglycan export | 20.3 (32.3) | Z22646 |
| | FeuC-Ba. subtilis | iron uptake | 17.1 (28.8) | L19954 |
| WbpG | ORF2-Vibrio cholerae O139 | unknown | 19.0 (23.7) | U47057 |
| | Pfk-Lactococcus lactis | phosphofructokinase | 9.7 (14.4) | L07920 |
| | NrfB-H. influenzae | formyl-dependent nitrate | 5.8 (9.3) | U32733 |
| | | reductase | | |
| WbpH | RfaK-Neisseria meningitidis | glycosyl transferase | 20.1 (28.9) | U35713 |
| | CapM-S. aureus | GalNAcA transferase | 17.4 (29.7) | U10927 |
| | IcsA-N. meningitidis | glycosyl transferase | 17.1 (27.0) | U39810 |
| | BplH-B. pertussis | glycosyl transferase | 16.6 (23.0) | X90711 |
| | BplE-B. pertussis | glycosyl transferase | 15.8 (24.6) | X90711 |
| WbpI | BplD-B. pertussis | GlcNAc to ManNAc epimerase | 56.6 (69.3) | X90711 |
| | EpsC-B. solanacearum | GalNAcA biosynthesis | 29.3 (42.3) | U17898 |
| | RffE (o389)-E. coli | UDP-GlcNAc-2-epimerase | 12.9 (18.8) | M87049 |
| | YvyH-Ba. subtilis | unknown | 12.3 (18.5) | P39131 |
| | RfbC-S. enterica sv Borreze | UDP-GlcNAc-2-epimerase | 11.8 (18.2) | L39794 |
| WbpJ | BplE-B. pertussis | glycosyl transferase | 39.5 (52.2) | X90711 |
| | TrsE-Yersinia enterocolitica O:3 | galactosyl transferase | 15.7 (26.7) | Z47767 |
| WbpK | ORF6-V. cholerae O139 | UDP-galactose-4-epimerase | 37.2 (53.8) | U47057 |
| | ExoB-R. meliloti | UDP-galactose-4-epimerase | 22.8 (32.8) | X58126 |
| | StrP-Str. glaucescens | dehydratase or epimerase | 22.5 (34.7) | X78974 |
| | RffG (o355)-E. coli | TDP-glucose dehydratase | 25.5 (38.1) | M87049 |
| | GraE-Str. violaceoruben | unknown | 21.3 (29.7) | L37334 |
| | RfbB-N. meningitidis | TDP-glucose dehydratase | 21.9 (31.6) | L09189 |
| | RfbB-E. coli | TDP-glucose dehydratase | 18.8 (28.5) | U23775 |
| WbpL | TrsF-Y. enterocolitica O3 | UDP-GalNAc transferase | 54.5 (67.7) | Z47767 |
| | Rfe-Mycobacterium leprae | UDP-GlcNAc transferase | 28.7 (46.5) | U15186 |
| | Rfe-M. tuberculosis | UDP-GlcNAc transferase | 28.5 (46.6) | Z73419 |
| | Rfe-E. coli | UDP-GlcNAc transferase | 19.8 (30.3) | M76129 |
| | Rfe-H. influenzae | UDP-GlcNAc transferase | 19.1 (29.7) | U32791 |
| WbpM | BplL-B. pertussis | dehydratase | 48.4 (59.6) | X90711 |
| | TrsG-Y. enterocolitica O3 | UDP-GalNAc biosynthesis | 48.1 (60.0) | Z47767 |
| | CapD-S. aureus | unknown | 39.2 (53.9) | U10927 |
| | ORF10-V. cholerae O139 | unknown | 32.5 (52.4)^{a} | U47057 |
| | ORF11-V. cholerae O139 | unknown | 52.7 (61.0)^{b} | U47057 |
| WbpN | NifV-Rhodobacter sphaeroides | homocitrate synthase | 19.2 (27.1) | Q01181 |

TABLE 3
Amino acid homologies of HisH proteins

| | PA | AB | EC | HI | LL | SC | ST |
|---|---|---|---|---|---|---|---|
| PA | 100.0 | — | — | — | — | — | — |
| AB | 53.6 | 100.0 | — | — | — | — | — |
| EC | 56.1 | 47.4 | 100.0 | — | — | — | — |
| HI | 51.8 | 47.9 | 63.3 | 100.0 | — | — | — |
| LL | 51.0 | 52.6 | 50.0 | 52.3 | 100.0 | — | — |
| SC | 54.9 | 47.9 | 55.1 | 45.2 | 48.0 | 100.0 | — |
| ST | 54.7 | 43.2 | 92.2 | 60.9 | 45.4 | 49.5 | 100.0 |
| Amino acid homologies of HisH proteins from various bacterial species. The amino acid sequences of various HisH proteins were aligned pairwise using the PC/GENE PALIGN program with the following parameters: K-tuple value = 1; gap penalty = 5; window size = 10; open gap cost = 10; unit gap cost = 10; filtering level = 2.5. The numbers shown are a summation of identical | | | | | | | |
| #and conserved amino acid residues. Key: PA, Pseudomonas aeruginosa O5 psb cluster HisH; AB,- Azo EC, Eseherichia coli HisH; HI, Haemophilus influenzae HisH; LL, Lactobacillus lactis HisH; RS, Rhodobacter sphaeroides HisH; and ST, Salmonella enterica typhimurium HisH. | | | | | | | |

TABLE 4
Amino acid homologies of HisF proteins.

| | Pa | Ab | Ec | Hi | Kp | Ll | Rs | St |
|---|---|---|---|---|---|---|---|---|
| Pa | 100.0 | — | — | — | — | — | — | — |
| Ab | 51.4 | 100.0 | — | — | — | — | — | — |
| Ec | 48.2 | 56.2 | 100.0 | — | — | — | — | — |
| Hi | 50.6 | 52.3 | 87.2 | 100.0 | — | — | — | — |
| Kp | 49.8 | 55.5 | 97.7 | 86.4 | 100.0 | — | — | — |
| Ll | 53.7 | 70.1 | 58.6 | 57.0 | 58.6 | 100.0 | — | — |
| Rs | 44.6 | 81.3 | 54.8 | 46.8 | 54.0 | 63.2 | 100.0 | — |
| St | 49.4 | 56.5 | 97.3 | 87.6 | 96.5 | 58.6 | 55.2 | 100.0 |
| Amino acid homologies of HisF proteins from various bacterial species. The amino acid sequences of various HisF proteins were aligned pairwise using the PC/GENE PALIGN program with the following parameters: K-tuple value = 1; gap penalty = 5; window size = 10; open gap cost = 10; unit gap cost = 10; filtering level = 2.5. The numbers shown are a | | | | | | | | |
| #summation of identical and conserved amino acid residues. Key: Pa, Pseudomonas aeruginosa O5 psb cluster HisF; Ab, Azo Escherichia coli HisF; Hi, Haemophilus influenzae HisF; Ll, Lactobacillus lactis, HisF; Rs, Rhodobacter sphaero4ides HisF; and St, Salmonella enterica typhimurium HisF. | | | | | | | | |

TABLE 5
Pairwise comparison of Rol amino acid homologies^{1,2}

| | PA | EC1 | EC2 | SF | ST |
|---|---|---|---|---|---|
| PA | 100.0 | 34.4 | 35.1 | 35.4 | 32.8 |
| EC1 | | 100.0 | 79.3 | 79.0 | 78.6 |
| EC2 | | | 100.0 | 98.1 | 81.5 |
| SF | | | | 100.0 | 81.2 |
| ST | | | | | 100.0 |

¹Analyses were done using PCGENE PALIGN program.
²PA, Pseudomonas aeruginosa O5 Rol; EC1, E. coli O75 Rol; EC2, E. coli O111 CLD; SF Shigella flexneri Rol; ST, Salmonella enterica serovar typhimurium strain LT2 CLD. Note tha CLD (chain length determinant) is another nomenclature used by some researchers (Bastin et al. 1993) to describe the same class of Rol proteins.

TABLE 6
Bacterial strains and plasmids

| Strain or plasmid | Genotype or relevant characteristics | Reference or source |
|---|---|---|
| P. aeruginosa | | |
| PAO1 | serotype O5, A⁺, B⁺ | Hancock and Carey (1979) |
| AK14O1 | mutant of OT684^{a}, A⁺, B-band contains core + | Berry and Kropinski (1986) |
| | one O-repeat unit (SR) | |
| rd7513 | mutant of AK14O1, A⁻, B-band contains core + | Lightfoot and Lam (1991) |
| | one O-repeat unit (SR) | |
| OP5.2 | mutant of PAO1, A⁺, B-band contains core + one | This study |
| | O-repeat unit (SR) | |
| OP5.3 | mutant of PAO1, A⁺, B-band contains core + one | This study |
| | O-repeat unit (SR) | |
| OP5.5 | mutant of PAO1, A⁺, B-band contains core + one | This study |
| | O-repeat unit (SR) | |
| E. coli | | |
| DH5n | supE44 hsdR17 recA1 endA1 gyrA96 | GIBCO/Bethesda Research |
| | thi-1 relA1 | Laboratories |
| HB101 | supE44 hsdS20(r⁻_{B} m⁻_{B}) recA13 ara-14 proA2 | Boyer and Roulland-Dussoix |
| | lacY1 galK2 rpsL20 xyl-5 mtl-1 | (1969) |
| | F⁻Str^{R} | |
| SM10 | thi-1 thr leu tonA lacY supE recA RP4-2-Tc::Mu | Simon et al. (1983) |
| | Km^{R} | |
| Plasmids | | |
| pFV100 | pCP13 derivative containing cloned PAO1 O- | Lightfoot and Lam (1993) |
| | antigen biosynthetic genes on a 26 kb insert | |
| pCP13 | RK2 derivative cos⁺, Mob⁺, Tra⁻, Tc^{R} Km^{R} | Darzins and Chakrabarty (1984) |
| pRK404 | RK2 derivative Mob⁺, Tra⁻, Tc^{R} | Ditta et al. (1985) |
| pUCP26 | pUC18-derived broad-host-range vector, Tc^{R} | West et al. (1994) |
| pEX100T | gene-replacement vector, ori T⁺, SacB⁺, Ap^{R} | Schweizer and Hoang (submitted) |
| pUCPGM | source of Gm^{R} cassette; Ap^{R} Gm^{R} | Schweizer (1993) |
| pBluescript KS | Ap^{R} | PDI Biosciences, Aurora, ON |
| (+/−) | | |

^{a}OT684 is the immediate progenitor strain of AK14O1 and is a restrictionless mutant of PAO1 (Potter and Loutit, 1982).

TABLE 7
Rfc proteins of P. aeruginosa and other gram-negative organisms

| | Total # | Mol. weight | Hydropathy | % | |
|---|---|---|---|---|---|
| Rfc protein | amino acids | (kD)^{a} | index^{b} | G + C^{c} | Reference |
| P aeruginosa | 438 | 48.9 | 0.8 | 44.8 | This study |
| S. enterica | 407 | 47.5 | 0.65 | 33.5 | Collins and Hackett |
| (typhimurium) | | | | | (1991) |
| S. enterica | 399 | 44.8 | 0.77 | 33.8 | Brown et al. (1992) |
| (muenchen) | | | | | |
| Shigella | 380 | 43.7 | 0.84 | 30.9 | Klena and |
| dysenteriae | | | | | Schnaitman (1993) |
| Shigella flexneri | 382 | 43.7 | 1.08 | 27.3 | Morona et al. (1994) |

^{a}Molecular weight based on nucleotide sequence.
^{b}Hydropathy index deduced from hydrophobicity analysis (Kyte and Doolittle, 1982).
^{c}Percentage of the bases G and C in the coding sequence.

TABLE 8
Bacterial strains and plasmids used in this study.

| Strain or plasmid | Genotype, phenotype or properties | Reference/source |
|---|---|---|
| P. aeruginosa | | |
| O5 | strain PAO1, wild type A+ B+ | 20 |
| O5 wzz | PAO1, wzz insertion mutation at XhoI; A+ B+ | this study |
| LATS O16 | Serotype O16 wild type A− B+ | 33 |
| O16 wzz | Serotype O16 wzz insertion mutation at XhoI; A− B+ | this study |
| E. coli | | |
| JM109 | recA1 supE44 endA1 hsdR17 gyrA96 relA1 thi (lac-proAB | 53 |
| | F′[tra D36, proAB+, lacIq, lacZ(M15] | |
| SM10 | thi-1 thr leu tonA lacY supE recA RP4-2-Tc::Mu, Km^{R} | 45 |
| HB101 | F- thi-1 hsdS20 serA ara14 proA2 lacY1 galK2 rpsL20 xyl | 27 |
| | mtl-1 supE44 recA13 leuB6 Str^{R} | |
| CLM4 | lacZ2286 trp-49 ((sbcB-rfb)86 upp-12 relA1 rps1150 (− recA | 35 |
| Plasmids | | |
| pFV100 | 24.4 kb XhoI fragment in cosmid pCP13; contains the wbp | 8, 31 |
| | cluster | |
| pFV400 | 25.0 kb Sau3A1 fragment in pCP13; overlaps pFV100 | this study |
| pFV401 | 2.3 kb HindIII fragment in pBluescript II SK; contains the | this study |
| | P. aeruginosa O5 wzz gene | |
| pFV401-26 | same insert in pUCP26 | this study |
| pFV401TGm | same insert in pEX100T, with Gm^{R} cassette inserted at | this study |
| | unique XhoI site within wzz | |
| pFV403 | 3.0 kb SstI fragment in pBluescript II SK; contains 5 | this study |
| | portion of wzz and upstream sequences | |
| pBluescript II SK | 2.9 kb cloning vector containing T7 promoter; Ap^{R} | Stratagene |
| pUCP26 | 4.9 kb pUC18-based broad-host-range vector; Tc^{R} | 48 |
| pEX100T | gene-replacement vector; oriT⁺, sacB⁺, Ap^{R} | 44 |
| pUCPGM | source of gentamicin resistance cassette; Ap^{R} , Gm^{R} | 44 |

TABLE 9
Amino acid identities/similarities of various wzz-like proteins.

| | Ec Wzz | Ec o349 | Sf Wzz | St Wzz | EcO8 Wzz | Ye Wzz | Yp Wzz | Ec FepE | Vc OtnB |
|---|---|---|---|---|---|---|---|---|---|
| Pa Wzz | 19.9 | 15.5 | 20.0 | 19.6 | 19.3 | 11.5 | 13.2 | 17.0 | 18.8 |
| | (33.4) | (26.5) | (35.4) | (32.8) | (32.9) | (19.0) | (23.3) | (27.3) | (30.4) |
| Ec Wzz | 100.0 | 25.1 | 65.5 | 64.8 | 65.2 | 19.3 | 22.6 | 26.9 | 18.7 |
| | | (35.8) | (79.0) | (78.6) | (80.4) | (27.3) | (35.4) | (39.4) | (28.4) |
| Ec o349 | — | 100.0 | 20.3 | 24.8 | 21.2 | 14.7 | 20.7 | 19.5 | 18.5 |
| | | | (32.0) | (37.6) | (33.9) | (22.7) | (31.9) | (31.3) | (26.3) |
| Sf Wzz | — | — | 100.0 | 72.0 | 88.9 | 15.7 | 20.9 | 24.6 | 18.8 |
| | | | | (81.2) | (93.6) | (25.9) | (33.5) | (36.6) | (25.0) |
| St Wzz | — | — | — | 100.0 | 71.2 | 15.6 | 22.6 | 26.6 | 22.6 |
| | | | | | (82.6) | (23.6) | (33.3) | (41.9) | (32.7) |
| Ec O8 Wzz | — | — | — | — | 100.0 | 15.2 | 15.5 | 24.7 | 15.2 |
| | | | | | | (26.0) | (26.9) | (36.1) | (26.3) |
| Ye Wzz | — | — | — | — | — | 100.0 | 37.3 | 25.1 | 10.4 |
| | | | | | | | (56.9) | (38.4) | (19.7) |
| Yp Wzz | — | — | — | — | — | — | 100.0 | 36.1 | 18.2 |
| | | | | | | | | (51.8) | (29.2) |
| Ec FepE | — | — | — | — | — | — | — | 100.0 | 14.0 |
| | | | | | | | | | (24.2) |

Numbers shown are percent identity, with percent similarity in brackets.
Pa, P. aeruginosa O5, accession U50397; Ec Wzz, E. coli O111, Z17241; Ec o349, E. coli, M87049; Sf Wzz, Shigella flexneri, X71970; St Wzz, S. enterica sv Typhimurium LT2, M89933; Ec O8 Wzz, E. coli O8, U39306; Ye Wzz, Yersinia enterocolitica O: 8, U43708; Yp Wzz, Y. pseudotuberculosis, U13685; Ec FepE, E. coli, P26266; Vc OtnB, Vibrio cholerae O139, X90547.

### REFERENCES

Alifano, P., Fani, R., Lió, P., Lazcano, A., Bazzicalupo, M., Stella Carlomagno, M., and Bruni, C. B. (1996) Histidine biosynthetic pathway and genes: structure, regulation, and evolution. Microbiol Rev 60: 44-69.

Allen and Maskell, (1996) The identification, cloning and mutagenesis of genetic locus required for lipopolysaccharide biosynthesis in Bordetella pertussis. Mol Microbiol 19: 37-52.

Altschul, S. E., G. Warren, W. Miller, E. U. Myers, and D. J. Lipman. 1990. Basic local alignment search tool. J. Mol. Biol. 215:403-410.

Amor, P., and L. Mutharia. (1995) Cloning and expression of rfb genes from Vibrio anguillarum serotype O2 in Escherichia coli: evidence for cross-reactive epitopes. Infect Immun 63: 3537-3542

Arisawa, A., Tsunekawa, H., Okamura, K. and Okamoto, R. (1995) Nucleotide sequence analysis of the carbomycin biosynthetic genes including the 3-O-acyltransferase gene from Streptomyces thermotolerans. Biosci Bioteclinol Biochem 59: 582-588.

Arsenault, T. L., Hughes, D. W., MacLean, D. B., Szarek, W. A., Kropinski, A. M. B. and Lam, J. S. 1991. Structural studies on the polysaccharide portion of “A-band” lipopolysaccharide from a mutant (AK14O1) of P. aeruginosa strain PAO1. Can J Chem 69: 1273-1280.

Bastin, D. A., G. Stevenson, P. K. Brown, A. Haase, and P. R. Reeves. 1993. Repeat unit polysaccharides of bacteria: a model for polymerization resembling that of ribosomes and fatty acid synthetase, with a novel mechanism for determining chain length. Mol. Microbiol. 7:725-734.

Batchelor, R. A., P. Alifano, E. Biffali, S. I. Hull, and R. A. Hull. 1992. Nucleotide sequences of the genes regulating O-polysaccharide antigen chain length (rol) from Escherichia coli and Salmonella typhimurium: Protein homology and functional complementation. J. Bacteriol. 174:5228-5236

Bechthold, A., Sohng, J. K., Smith, T. M., Chu, X. and Floss, H. G. (1995) Identification of Streptomyces violaceoruber Tu22 genes involved in the biosynthesis of granaticin. Mol Gen Genet 248: 610-620.

Berry, D., and Kropinski, A. M. 1986. Effect of lipopolysaccharide mutations and temperature on plasmid transformation efficiency in P. aeruginosa. Can J Microbiol 32:436-438.

Bik, E. M., A. E. Bunschoten, R. J. L. Willems, A. C. Y. Chang, and F. R. Mooi. 1996. Genetic organization and functional analysis of the otn DNA essential for cell-wall polysaccharide synthesis in Vibrio cholerae O139. Mol. Microbiol. 20:799-811.

Binotto, J., MacLachlan, R., and Sanderson, K. E. 1991. Electrotransformation in Salmonella typhimurium LT2. Can J Microbiol 37:474-477.

Birnboim, H. C., and Doly, J. 1979. A rapid extraction procedure for screening recombinant plasmid. Nucleic Acids Res. 7:1513-1523.

Bogdanova, N., Bork, C., and Hell, R. (1995) Cysteine biosynthesis in plants: isolation and functional identification of a cDNA encoding a serine acetyltransferase from Arabidopsis thaliana. FEBS Lett 358: 43-47.

Boyer, H. W., and Roulland-Dussoix, D. 1969. A complementation analysis of the restriction and modification of DNA in Escherichia coli. J Mol Biol 41:459-496.

Brown, P. K., Romana, L. K., and Reeves, P. R. 1992. Molecular analysis of the rfb gene cluster of Salmonella serovar muenchen (strain M67), the genetic basis of the polymorphism between groups C2 and B. Mol Microbiol 6:1385-1394.

Buendia, A. M., Enenkel, B., Koplin, R., Niehaus, K., Arnold W., and Pühler, A.. (1991) The Rhizobium meliloti exoZ/exoB fragment of megaplasmid 2: ExoB functions as a UDP-glucose-4-epimerase and ExoZ shows homology to NodX of Rhizobium leguminosarum biovar viciae strain TOM. Mol Microbiol 5: 1519-1530.

Burnette, W. N. 1981. Western blotting: electrophoretic transfer of proteins from sodium dodecyl sulphate-polyacrylamide gels to unmodified nitrocellulose and radiographic detection with antibody and radioiodinated protein A. Anal. Biochem. 112:195-203.

Burrows, L. L., D. Chow, and J. S. Lam. 1997. Pseudomonas aeruginosa B-band O antigen chain length is modulated by Wzz (Rol). J. Bacteriol. 179: in press.

Burrows, L. L., D. F. Charter, and J. S. Lam. 1996. Molecular characterization of the Pseudomonas aeruginosa serotype O5 B-band lipopolysaccharide gene cluster. Mol. Microbiol. 22:481-495.

Collins, L. V., and Hackett, J. 1991. Molecular cloning, characterization, and nucleotide sequence of the rfc gene, which encodes an O-antigen polymerase of Salmonella typhimurium. J Bacteriol 173:2521-2529.

Comstock, L. E., Johnson, J. A., Michalski, J. M., Morris, J. G., Jr., and Kaper, J. P. (1996) Cloning and sequence of a region encoding a surface polysaccharide of Vibrio cholerae O139 and characterization of the insertion site in the chromosome of Vibrio cholerae O1. Mol Microbiol 19: 815-826.

Cryz, S. J. Jr., T. L. Pitt, E. Furer, and R. Germanier. 1984. Role of lipopolysaccharide in virulence of Pseudomonas aeruginosa. Infect. Immun. 44:508-513.

Daniels, D. L., Plunkett, G., Burland, V., and Blattner, F. R. (1992) Analysis of the Escherichia coli genome: DNA sequence of the region from 84.5 to 86.5 minutes. Science 257: 771-778.

Darzins, A., and Chakrabarty, A. M. 1984. Cloning of genes controlling alginate biosynthesis from a mucoid cystic fibrosis isolate of P. aeruginosa. J Bacteriol 159:9-18.

Dasgupta, T., and Lam, J. S. Identification of putative rfb genes involved in B-band lipopolysaccharide biosynthesis in P. aeruginosa serotype O5. Submitted for publication.

Dasgupta, T., and J. S. Lam. (1995) Identification of rfbA, involved in B-band lipopolysaccharide biosynthesis in Pseudomonas aeruginosa serotype O5. Infection and Immunity 63: 1674-1680.

Dasgupta, T., Malburg, S., and Lam, J. S. 1993. Program Abstr 93rd Gen Meet Amer Soc Microbiol abstr. D-240.

Davis, E. O., Evans, I. J. and Johnston, A. W. (1988) Identification of nodX, a gene that allows Rhizobium leguminosarum biovar viciae strain TOM to nodulate Afghanistan peas. Mol Gen Genet 212: 531-535.

Denk, D. and Bock, A. (1987) L-cysteine biosynthesis in Escherichia coli: nucleotide sequence and expression of the serine acetyltransferase (cysE) gene from the wild-type and a cysteine-excreting mutant. J Gen Microbiol 133: 515-525.

de Kievit, T. R., T. Dasgupta, H. Schweitzer, and J. S. Lam. 1995. Molecular cloning and characterization of the rfc gene of Pseudomonas aeruginosa (serotype O5). Mol. Microbiol. 16:565-574.

de Kievit, T. R., and J. S. Lam. 1997. Pseudomonas aeruginosa rfc genes of serotypes O2 and O5 could complement O-polymerase deficienct SR mutants of either serotype. FEMS Microbiol. Letters, in press.

de Kievit, T. R., and Lam, J. S. 1994. Program Abstr 94th Gen Meet Amer Soc Microbiol abstr. D-192.

de Kievit, T. R., Dasgupta, T., Schweizer, H., and Lam; J. S. (1995) Molecular cloning and characterization of the rfc gene of Pseudomonas aeruginosa (serotype O5). Mol Microbiol 16: 565-574.

de Lencastre, H., Chak, K.-F., and Piggot, P. J. 1983. Use of Escherichia coli transposon Tn1000 (γδ) to generate mutations in Bacillus subtilis DNA. J Gen Microbiol 129:3202-3210.

Delic-Attree, I., B. Toussaint, and P. M. Vignais. 1995. Cloning and sequence analyses of the genes coding for the integration host factor (IHF) and HU proteins of Pseudomnonas aeruginosa. Gene 154:61-64.

Deretic, V., Gill, J. F., and Chakrabarty, A. M. (1987) Gene algD coding for GDPmannose dehydrogenase is transcriptionally activated in mucoid Pseudomonas aeruginosa. J Bacteriol 169: 351-358.

Dhillon, N., Hale, R. S., Cortes, J., and Leadlay, P. F. (1989) Molecular characterization of a gene from Saccharopolyspora erythraea (Streptomyces erythraeus) which is involved in erythromycin biosynthesis. Mol Microbiol 3: 1404-1414.

Ditta, G., Schmidhauser, T., Yakobson, E., Su, P., Liang, X.-W., Finlay, D. R., Guiney, D., and Helinski, D. R. 1985. Plasmids related to the broad host range vector, pRK290, useful for gene cloning and for monitoring gene expression. Plasmid 13:149-153.

Dodgson, C., P. Amor, and C. Whitfield. 1996. Distribution of the rol gene encoding the regulator of lipopolysaccharide O-chain length in Escherichia coli and its influence on the expression of group I capsular K antigens. J. Bacteriol. 178:1895-1902.

Dodgson, C., P. Amor, and, C. Whitfield. 1996. Distribution of the rol gene encoding the regulator of lipopolysaccharide O-chain length in Escherichia coli and its influence on the expression of group I capsular K antigens. J. Bacteriol. 178:1895-1902.

Dubray, G., and G. Bezard. 1982. A highly sensitive periodic acid-silver stain for 1,2-diol groups of glycoproteins and polysaccharides in polyacrylamide gels. Anal Biochem 119:325-329.

Falah, M. and R. S. Gupta. 1994. Cloning of the hsp70 (dnaK) genes from Rhizobium meliloti and Pseudomonas cepacia: phylogenetic analyses of mitochondrial origin based on a highly conserved protein sequence. J Bacteriol 176: 7748-7753.

Farinha, M. A., and Kropinski, A. M. 1990. High efficiency electroporation of P. aeruginosa using frozen cell suspensions. FEMS Microbiol Lett 70:221-226.

Fleischmann, R. D., Adams, M. D., White, O., Clayton, R. A., Kirkness, E. F., Kerlavage, A. R. Bult, C. J., Tomb, J.-F., Dougherty, B. A., Merrick, J. M., McKenney, K., Sutton, G.,,FitzHugh, W., Fields, C. A., Gocayne, J. D., Scott, J. D., Shirley, R., Liu, L. -I., Glodek, A., Kelley, J. M., Weidman, J. F., Phillips, C. A., Spriggs, T., Hedblom, E., Cotton, M.. D., Utterback, T. R., Hanna, M. C., Nguyen, D. T., Saudek, D.. M., Brandon, R. C., Fine, L. D., Fritchman, J. L., Fuhrmann, J. L., Geoghagen, N. S. M., Gnehm, C. L., McDonald, L. A., Small, K. V., Fraser, C. M., Smith, H. O. and Venter, J. C. (1995) Whole-genome random sequencing and assembly of Haemophilus influenzae Rd. Science 269: 496-512.

Franco, A. V., D. Liu, and P. R. Reeves. 1996. A Wzz (Cld) protein determines the chain length of K lipopolysaccharide in Escherichia coli O8 and O9 strains. J. Bacteriol. 178:1903-1907.

Gagnon, Y., Breton, R., Putzer, H., Pelchat, M., Grunberg-Manago, M., and Lapointe, J. (1994) Clustering and co-transcription of the Bacillus subtilis genes encoding the aminoacyl-tRNA synthetases specific for glutamate and for cysteine and the first enzyme for cysteine biosynthesis. J Biol Chem 269: 7473-7482.

Gish, W., and D. J. States. 1993. Identification of protein coding regions by database similarity search. Nature Genet. 3:266-272.

Glaser, P., Kunst, F., Arnaud, M., Coudart, M.-P., Gonzales, W., Hullo, M.-F., Ionescu, M., Lubochinsky, B., Marcelino, L., Moszer, I., Presecan, E., Santana, M., Schneider, E., Schweizer, J., Vertes, A., Rapoport, G., and Danchin, A.. (1993) Bacillus subtilis genome project: cloning and sequencing of the 97 kb region from 325° to 333°. Mol Microbiol 10: 371-384.

Glucksmann, M. A., Reuber, T. L., Walker, G. C. (1993) Genes needed for the modification, polymerization, export, and processing of succinoglycan by Rhizobiiim meliloti: a model for succinoglycan biosynthesis. J Bacteriol 175: 7045-7055.

Göhmann, S., Manning, P. A., Alpert, C. A., Walker, M. J., and Timmis, K. N. (1994) Lipopolysaccharide O-antigen biosynthesis in Shigella dysenteriae serotype 1: analysis of the plasmid-carried rfp determinant. Microb Pathog 16: 53-64

Gold, L., and Stormo, G., (1987) Transcriptional initiation. In Escherichia coli and Salmonella typhimurium: Cellular and Molecular Biology. Vol. 2. Neidhardt, F. C (ed). Washington, D.C. American Society for Microbiology, pp.807-876.

Goldberg, J. B., K. Hatano, G. Small Meluleni, and G. B. Pier. 1992. Cloning and surface expression of Pseudomonas aeruginosa O antigen in Escherichia coli. Proc. Nat. Acad. Sci USA 89:10716-10720.

Goldberg, J. B., and D. E. Ohman. 1984. Cloning and expression in Pseudomonas aeruginosa of a gene involved with the production of alginate. J. Bacteriol. 158:1115-1121.

Goldberg, J. B., K. Hatano, G. Small Meluleni, and G. B. Pier. 1992. Cloning and surface expression of Pseudomonas aeruginosa O antigen in Escherichia coli. Proc. Nat. Acad. Sci USA 89:10716-10720.

Goldman, R. C., and L. Leive. 1980. Heterogeneity of antigenic-side-chain length in lipopolysaccharide from Escherichia coli O111 and Salmonella typhimurium LT2. Eur. J. Biochem. 107:145-153.

Gotschlich, 1994.

Hammerschmidt, S., Birkholz, C., Zahringer, U., Robertson, B. D., van Putten, J., Ebelling, O., and Frosch, M., (1994) Contribution of genes from the capsule gene complex (cps) to lipooligosaccharide biosynthesis and serum resistance in Neisseria meningitidis. Mol Microbiol 11: 885-896.

Hancock, R. E. W., and A. M. Carey. 1979. Outer membrane of Pseudomonas aerziginosa: heat- and 2-mercaptoethanol-modifiable proteins. J. Bacteriol. 158: 1115-1121.

Harley, C. B. and R. P. Reynolds (1987) Analysis of E. coli promoter sequences. Nucleic Acids Res 15: 2343-2361.

Hashimoto, Y., Li, N., Yokoyama, H. and Ezaki, T. (1993) Complete nucleotide sequence and molecular characterization of ViaB region encoding Vi antigen in Salmonella typhi. J Bacteriol 175: 4456-4465.

Hitchcock, P. J., and T. M. Brown. 1983. Morphological heterogeneity among Salmonella lipopolysaccharide chemotypes in silver-stained polyacrylamide gels. J. Bacteriol. 154:269-277.

Holloway, B. W., Römling, U., Tümmler, B. (1994) Genomic mapping of Pseudomonas aeruginosa PAO. Microbiology 140: 2907-2929.

Holloway, B. W., U. Rmling, and B. Tmmler. 1994. Genomic mapping of Pseudomonas aeruginosa PAO. Microbiology 140:2907-2929.

Huang, J., and Schell, M. (1995). Molecular characterization of the eps gene cluster of Pseudoinonas solanacearum and its transcriptional regulation at a single promoter. Mol Microbiol 16: 977-989.

Huff, J. P., B. J. Grant, C. A. Penning, and K. F. Sullivan. 1990. Optimization of routine transformation of Escherichia coli with plasmid DNA. Biotechniques 9:570-577.

Jarosik, G. P. and E. J. Hansen. 1994. Identification of a new locus involved in expression of Haemophilus influenzae type b lipooligosaccharide. Infect Immun 62: 4861-4867.

X. M. Jiang, B. Neal, F. Santiago, S. J. Lee, L. K. Romana & P. R. Reeves (1991). Structure and sequence of the rib (O antigen) gene cluster of Salmonella serovar typhimurium (strain LT2).Mol Microbiol 5: 695-713.

Kao, C. C. and L. Sequeira 1991. A gene cluster required for coordinated biosynthesis of lipopolysaccharide and extracellular polysaccharide also affects virulence of Pseudomonas solanacearum. J Bacteriol 173: 7841-7847.

Kearney, B., and Staskawicz, B. J. (1990) Characterization of IS476 and its role in bacterial spot disease of tomato and pepper. J Bacteriol 172: 143-148.

Keenleyside W. J., M. Perry, L. Maclean, C. Poppe and C. Whitfield. 1994. A plasmid-encoded rfr O:54 gene cluster is required for biosynthesis of the O:54 antigen in Salmonella enterica serovar Borreze. Mol Microbiol 11: 437-448.

Keenleyside, W. J., and Whitfield, C. (1995) Lateral transfer of rfb genes: a mobilizable ColE1-type plasmid carries the rfb O:54 (O:54 antigen biosynthesis) gene cluster from Salmonella enterica serovar Borreze. J Bacteriol 177: 5247-5253.

Keenleyside, W. J., and C. Whitfield. 1996. A novel pathway for O-polysaccharide biosynthesis in Salmonella enterica serovar Borreze. J. Biol. Chem. 271:28581-28592.

Kingsley, M. T., D. W. Gabriel, G. C. Marlow & P. D. Roberts. 1993. The ops X locus of Xanthonionas campestris affects host range and biosynthesis of lipopolysaccharide and extracellular polysaccharide. J Bacteriol 175: 5839-50.

Klein, P., Kanehisa, M., and DeLisi, C. 1985. Description of one of the methods used in SOAP. Biochimica et Biophysica Acta 815:468-476.

Klena, J. D., and Schnaitman, C. A. 1993. Function of the rib gene cluster and the rfe gene in the synthesis of O-antigen by Shigella dysenteriae 1. Mol Microbiol 9:393-402.

Knirel, Y. A. 1990. Polysaccharide antigens of P. aeruginosa. Crit Rev Microbiol 17:273-304.

Knirel, Y. A., and N. K. Kochetkov. 1994. The structure of lipopolysaccharides of Gram-negative bacteria. III. The structure of O-antigens: a review. Biochemistry (Moscow) 59:1325-1383.

Knirel, Y. A., E. V. Vinogradov, N. A. Kocharova, N. A. Paramonov, N. K. Kochetkov, B. A. Dmitriev, E. S. Stanislavsky, and B. Lanyi. 1988. The structure of O-specific polysaccharides and the serological classification of Pseudomonas aeruginosa. Acta Microbiol. Hung. 35:3-24.

Kuenzler, M., Balmelli, T., Egli, C. M., Paravicini, G., and Braus, G. H. (1993) Cloning, primary structure, and regulation of the HIS7 gene encoding a bifunctional glutamine amidotransferase: cyclase from Saccharomyces cerevisiae. J Bacteriol 175: 5548-5558.

Kuzio, J., and Kropinski A. M. (1983) O-antigen conversion in Pseudomonas aeruginosa PAO1 by bacteriophage D3. J Bacteriol 155: 203-212

Lacks, S., and J. R. Greenberg. 1977. Complementary specificity of restriction endonucleases of Diplococcus pneumoniae with respect to DNA methylation. J. Mol. Biol. 114: 153-168.

Lam, M. Y. C., E. J. McGroarty, A. M. Kropinski, L. A. MacDonald, S. S. Pedersen, N. Hiby, and J. S. Lam. 1989. Occurrence of a common lipopolysaccharide antigen in standard and clinical strains of Pseudomonas aeruginosa. J. Clin. Microbiol. 27:962-967.

Lam, J. S., M. Y. C. Handelsman., T. R. Chivers, and L. A. MacDonald. 1992. Monoclonal antibodies as probes to examine serotype-specific and cross-reactive epitopes of lipopolysaccharides from serotypes O2, O5, and O16 of Pseudomonas aeruginosa. J. Bacteriol. 174:2178-2184.

Lai, C.-Y. and Baumann, P. (1992) Sequence analysis of a DNA fragment from Buchnera aphidicola (an endosymbiont of aphids) containing genes homologous to dnaG, rpoD, cysE, and secB. Gene 119: 113-118.

Lightfoot, J. L., and J. S. Lam. 1991. Molecular cloning of genes involved with expression of A-band lipopolysaccharide, an antigenically conserved form, in Pseudomonas aeruginosa. J. Bacteriol. 173:5624-5630.

Lightfoot, J. L., and J. S. Lam. 1993. Chromosomal mapping, expression and synthesis of lipopolysaccharide in Pseudomonas aeruginosa: a role for guanosine diphospho (GDP)-D-mannose. Mol. Microbiol. 8:771-782.

Liu, D., R. A. Cole, and P. R. Reeves. 1996. An O-antigen processing function for Wzx (RfbX): a promising candidate for O-unit flippase. J. Bacteriol. 178:2102-2107.

Liu, P. V. and S. Wang. 1990. Three new major somatic antigens of Pseudomonas aeruginosa. J. Clin. Microbiol. 28:922-925.

Lin, W. S., Cunneen, T. and Lee, C. Y. (1994) Sequence analysis and molecular characterization of genes required for the biosynthesis of type 1 capsular polysaccharide in Staphylococcus aureus. J Bacteriol 176: 7005-7016.

Liu, P. V., Matsumoto, H., Kusama, H., and Bergan, T. 1983. Survey of heat-stable major- somatic antigens of P. aeruginosa. Int J Syst Bacteriol 33:256-264.

Macpherson, D. F., Manning, P. A., and Morona, R. (1994) Characterization of the dTDP rhamnose biosynthethic genes encoded in the rfb locus of Shigella flexneri. Mol Microbiol 11: 281-292.

MacLachlan, P. R., S. K. Kadam, and K. E. Sanderson. 1991. Cloning, characterization, and DNA sequence of the rfaLK region for lipopolysaccharide synthesis in Salmonella typhimurium LT2. J. Bacteriol. 173:7151-7163.

Mäkelä, P. H., and Stocker, B. A. D. 1984. Genetics of lipopolysaccharide, p. 59-137. In E. T. Rietschel (ed.), Handbook of endotoxin, vol. 1. Elsevier Science Publishing, Amsterdam.

Marolda, C. L., and M. A. Valvano. 1993. Identification, expression, and DNA sequence of the GDP-manose biosynthesis genes encoded by the O7 rfb cluster of strain VW187 (Escherichia coli O7:K1). J. Bacteriol. 175:148-158.

Marolda, C. L., and Valvano, M. A. (1995) Genetic analysis of the dTDP-rhamnose biosynthesis region of the Escherichia coli VW187 (O7:K1) rfb gene cluster: identification of functional homologs of rfbB and rfbA in the rff cluster and correct location of the rffE gene. J Bacteriol 177: 5539-5546.

May, T. B., D. Shinabarger, R. Maharaj, J. Kato, L. Chu, J. D. DeVault, S. Roychoudhury, N. A. Zielinski, A. Berry, R. K. Rothmel, T. K. Misra, and A. M. Chakrabarty. 1991. Alginate synthesis by Pseudomonas aeruginosa: a key pathogenic factor in chronic pulmonary infections of cystic fibrosis patients. Clin. Microbiol. Rev. 4:191-206.

Meier-Dieter, U., Barr, K., Starman, R., Hatch, L. and Rick, P. D. (1992) Nucleotide sequence of the Escherichia coli rfe gene involved in the synthesis of enterobacterial common antigen: Molecular cloning of the rfe-rff gene cluster. J Biol Chem 267: 746-753.

Morona, R., Mavris, M., Fallarino, A., and Manning, P. A. 1994. Characterization of the rfc region of Shigella flexneri. J Bacteriol 176: 733-747.

Morona, R., L. van den Bosch, and P. A. Manning. 1995. Molecular, genetic, and topological characterization of O-antigen chain length regulation in Shigella flexneri. J Bacteriol 177:1059-1068.

Nurminen, M., Hellerqvist, C. E., Valtonen, V. V., and Mäkelä, P. H. 1971. The smooth lipopolysaccharide character of 1, 4, (5), 12 and 1, 9, 12 transductants formed as hybrids between groups B and D of Salmonella. Eur J Biochem 22: 500-505.

Ogasawara, N., Nakai, S. and Yoshikawa, H. (1994) Systematic sequencing of the 180 kilobase region of the Bacillus subtilis chromosome containing the replication origin. DNA Res 1: 1-14.

Ozenberger, B. A., M. Schrodt Nahlik, and M. A. McIntosh. 1987. Genetic organization of multiple fep genes encoding ferric enterobactin transport functions in Escherichia coli. J. Bacteriol. 169:3638-3646.

Palleroni, N. J. 1984. Genus I. P.. p. 141-199. In N. R. Krieg and J. C. Holt. (ed.), Bergey's Manual of Systematic Bacteriology, Vol. 1, Williams and Wilkins, Baltimore.

Peschke, U., Schmidt, H., Zhang, H. Z. and Piepersberg, W. (1995) Molecular characterization of the lincomycin-production gene cluster of Streptomyces lincolnensis 78-11. Mol Microbiol 16: 1137-1156.

Potter, A. A. and Loutit, J. S. 1982. Exonuclease activity from P. aeruginosa which is missing in phenotypically restrictionless mutants. J Bacteriol 151: 1204-1209.

Prère, M. F., Chandler, M., and Fayet, O. (1990) Transposition in Shigella dysenteriae: isolation and analysis of IS911, a new member of the IS3 group of insertion sequences. J Bacteriol 172: 4090-4099.

Priefer, U. B., Kalinowski, J., Ruger, B., Heumann, W., and Puhler, A. (1989) ISR1, a transposable DNA sequence resident in Rhizobium class IV strains, shows structural characteristics of classical insertion elements. Plasmid 21: 120-128.

Pritchard, A. E., and Vasil, M. L. (1990) Possible insertion sequences in a mosaic genome organization upstream of the exotoxin A gene in Pseudomonas aeruginosa. J Bacteriol 172: 2020-2028.

Quirk, P. G., Guffanti, A. A., Clejan, S., Cheng, J., and Krulwich, T. A. (1994) Isolation of Tn917 insertional mutants of Bacillus subtilis that are resistant to the protonophore carbonyl cyanide m-chlorophenylhydrazone. Biochim Biophys Acta 1186: 27-34.

Reeves, P. (1993) Evolution of Salmonella O antigen variation by interspecific gene transfer on a large scale. Trends Genet 9: 17-222.

Reeves, P. R., M. Hobbs, M. Valvano, M. Skurnik, C. Whitfield, D. Coplin, N. Kido, J. Klena, D. Maskell, C. Raetz, and P. Rick. 1996. Proposal for a new nomenclature for bacterial surface polysaccharide genes. Trends Microbiol. 4: 495-503.

Rieder, B., Merrick, M. J., Castorph, H., Kleiner, D. (1994) Function of hisF and hisH gene products in histidine biosynthesis. J Biol Chem 269: 14386-14390.

Rivera, M., Bryan, L. E., Hancock, R. E. W. and McGroarty, E. J. 1988. Heterogeneity of lipopolysaccharides from P. aeruginosa: analysis of lipopolysaccharide chain length. J Bacteriol 170:512-521.

Rivera, M., T. R. Chivers, J. S. Lam, and E. J. McGroarty. 1992. Common antigen lipopolysaccharide from Pseudomonas aeruginosa AK1401 as a receptor for bacteriophage A7. J. Bacteriol. 174:2407-2411.

Rossbach, S., D. A. Kulpa, U. Rossbach and F. J. de Bruijn (1994) Molecular and genetic characterization of the rhizopine catabolism (mocABRC) genes of Rhizobium meliloti L5-30. Mol Gen Genet 245: 11-24.

Ruvkun, G. B., and Ausubel, F. M. 1981. A general method for site-directed mutagenesis in prokaryotes. Nature (London) 289:85-88.

Schnaitman, C. A., and J. D. Klena. 1993. Genetics of lipopolysaccharide biosynthesis in enteric bacteria. Microbiol. Rev. 57: 655-682.

Schnier, J., M. Kimura, K. Foulaki, A. R. Subramanian, K. Isono, and B. Wittmann-Liebold. 1982. Primary structure of Escherichia coli ribosomal protein Si and of its gene rpsA. Proc. Natl. Acad. Sci. U.S.A. 79:1008-1011.

Schweizer, H. P. 1993. Small broad-host-range gentamycin resistance gene cassettes for site-specific insertion and deletion mutagenesis. BioTechniques 15:831-833.

Schweitzer, H. P., and T. T. Hoang. 1995. An improved system for gene replacement and xylE fusion analysis in Pseudomonas aeruginosa. Gene 158:15-22.

Segal G. and E. Z. Ron (1995) The dnaKJ operon of Agrobacterium tumefaciens: transcriptional analysis and evidence for a new heat shock promoter J Bacteriol 177: 5952-5958.

Simon, R., Priefer, U., and Pühler, A. 1983. A broad-host-range mobilization system for in vivo genetic engineering: transposon mutagenesis in gram negative bacteria. Bio/Technology 1:784-791.

Skurnik, M., Venho, R., Toivanen, P., and Alhendy, A. (1995). A novel locus of Yersinia enterocolitica serotype O:3 involved in lipopolysaccharide outer core biosynthesis. Mol Microbiol 17: 575-594.

Sokol, P. A., Luan, M. Z., Storey, D. G., and Thirukkumaran, P. (1994) Genetic rearrangement associated with in vivo mucoid conversion of Pseudomonas aeruginosa PAO is due to insertion elements. J Bacteriol 176: 553-562.

Soldo, B., Lazarevic, V., Margot, P., and Karamata, D. (1993) Sequencing and analysis of the divergon comprising gtaB, the structural gene of UDP-glucose pyrophosphorylase of Bacillus subtilis 168. 1 J Gen Microbiol 139: 3185-3195.

Stutzman-Engwall, K. J., Otten, S. L., and Hutchinson, C. R. (1992) Regulation of secondary metabolism in Streptomyces spp. and overproduction of daunorubicin in Streptomyces peucetius. J Bacteriol 174: 144-154.

Sturm, S. and K. N. Timmis. 1986. Cloning of the rfb region of Shigella dysenteriae 1 and construction of an rfb-rfp gene cassette for the development of lipopolysaccharide-based live anti-dysentery vaccines. Microb. Pathog. 1:289-297.

Tabor, S., and C. C. Richardson. 1985. A bacteriophage T7 RNA polymerase/promoter system for controlled exclusive expression of specific genes. Proc. Nat. Acad. Sci. USA 82:1074-1078.

Takagi, M., Takada, H., and Imanaka, T. (1990) Nucleotide sequence and cloning in Bacillus siibtilis of the Bacillus stearothermophilus pleiotropic regulatory gene degT. J Bacteriol 172: 411-418.

Tercero, J. A., Espinosa, J. C., Lacalle, R. A. and Jimenez, A. (1996) The biosynthetic pathway of the aminonucleoside antibiotic puromycin, as deduced from the molecular analysis of the pur cluster of Streptomyces alboniger. J Biol Chem 271: 1579-1590.

Thorson, J. S., Lo, S. F., Ploux, O., He, X., and Liu, H. -W. (1994) Studies of the biosynthesis of 3,6-dideoxyhexoses: molecular cloning and characterization of the asc (ascarylose) region from Yersinia pseudotuberculosis serogroup VA. J Bacteriol 176: 5483-5493.

West, S. E. and Iglewski, B. H. (1988) Codon usage in Pseudomonas aeruginosa. Nucleic Acids Res 16: 9323-9335.

West, S. E. H., H. P. Schweizer, C. Dall, A. K. Sample, and L. J. Runyen-Janecky. 1994. Construction of improved Escherichia-Pseudomonas shuttle vectors derived from pUC18/19 and the sequence of the region required for their replication in Pseudomonas aeruginosa. Gene 128: 81-86.

West, S. E. H., Schweizer, H. P., Dall, C., Sample, A. K., and Runyen-Janecky, L. J. (1994) Construction of improved Escherichia-P. shuttle vectors derived from pUC18/19 and the sequence of the region required for their replication in P. aeruginosa. Gene 128:81-86.

Whitfield, C. 1995. Biosynthesis of lipopolysaccharide O-antigens. Trends Microbiol. 3:178-185.

Whitfield, C., and M. A. Valvano. 1993. Biosynthesis and expression of cell-surface polysaccharides in gram-negative bacteria. Adv. Microb. Physiol. 35:135-246.

Wozniak, D. J. 1994. Integration host factor and sequences downstream of the Pseudomonas aeruginosa algD transcription start site are required for expression. J. Bacteriol. 176:5068-5076.

Wozniak, D. J., and D. E. Ohman. 1993. Involvement of the alginate algT gene and integration host factor in the regulation of the Pseudomonas aeruginosa algB gene. J Bacteriol 175: 4145-4153.

Wood, M. S., Byrne, A., and Lessie, T. G. (1991) IS406 and IS407, two gene-activating insertion sequences from Pseudomonas cepacia. Gene 105: 101-105.

Xiao, Q. and Moore, C. H. (1993) The primary structure of phosphofructokinase from Lactococcus lactis. Biochem Biophys Res Commun 194: 65-71.

Yanisch-Perron, C., J. Vieira, and J. Messing. 1985. Improved M13 phage cloning vectors and host strains: nucleotide sequences of the M13mp18 and pUC19 vectors. Gene 33:103-119

Detailed Figure Legends for FIGS. 22 to 29, 32, 33, and 43 to 47

FIG. 22. Silver-stained SDS-PAGE gel of LPS from PAO1, AK14O1, AK14O1(pFV100), and AK14O1(pFV.TK8) (Panel A) and Westernimmunoblots of this LPS reacted with O5-specific MAb MF15-4 (Panel B). Note that the two transconjugants strains, AK14O1(pFV100) and AK14O1(pFV.TK8), produce levels of B-band LPS similar to the PAO1 wild-type strain.

FIG. 23. Restriction maps of the chromosomal inserts from pFV100 and several pFV subclones. Results of complementation studies of the SR mutants AK14O1 and rd7513 with the pFV subclones are also shown. The three Tn1000 insertions in the 1.5 kb XhoI fragment of pFV.TK6 that were found to interrupt O-antigen complementation in AK14O1 are indicated. This XhoI fragment was later purified and used as a probe in Southern blot analysis. Restriction sites: B, BamHI; X, XhoI; S, SpeI; Xb, XbaI; H, HindIII.

FIG. 24. Southern analysis the three rfc chromosomal mutants, OP5.2, OP5.3, and OP5.5, showing the insertion of an 875 bp Gm^{R} cassette into the rfc gene. Restriction maps of the PAO1 wild-type (panel A) and mutant (panel B) rfc coding regions are shown. Southern hybridizations of chromosomal DNA from PAO1 (lane 1) and mutants OP5.2, OP5.3, and OP5.5 (lanes 2-4, respectively) digested with XhoI were performed using an rfc probe (panel C). This DIG-labelled probe was generated from the 1.5 kb XhoI insert of pFV.TK7 (shown in panel A). The probe hybridized to a 1.5 kb fragment of PAO1 and a 2.4 kb fragment of the three rfc mutants. The molecular size of the probe-reactive fragments are shown on the left (in kb).

FIG. 25. Silver-stained SDS-PAGE gel and Western blots of LPS from PAO1, AK14O1 and the three rfc chromosomal mutants, OP5.2, OP5.3, and OP5.5. Panel A: silver-stained SDS-PAGE gel; Panel B: Western blot reacted with O5-specific MAb MF15-4; Panel C: Western blot reacted with A-band specific MAb N1F10. Note that the chromosomal rfc mutants are not able to produce long-chain O-antigen; however, they are still expressing A-band LPS, like the SR mutant AK14O1.

FIG. 26. Restriction maps of recombinant plasmids pFV161, pFV401 and pFV402. The shaded box represents the DIG-labeled probe generated from pFV161. Restriction sites: B, BamHI; H, HindIII; X, XhoI.

FIG. 27. Southern hybridizations of chromosomal DNA from PAO1 (lane 2) and rol mutants (lanes 3&4). Chromosomal DNA in Panel A was digested with PstI and SstI. DNA in Panel B was digested with HindIII. The samples in Panel A were probed with the Gm^{R} cassette (Schweizer, 1993). The probe used in Panel B is the 2.3 kb HindIII insert from pFV401. Molecular weight markers, using λ DNA digested with HindIII, are indicated to the left of each panel.

FIG. 28. Characterization of LPS from PAO1 and PAO1 rol chromosomal mutants. The samples in each lane are as labeled. Panel A is a silver-stained SDS-PAGE gel. Panel B is the corresponding Western immunoblot reacted with an O5 (B-band)-specific mAb MF15-4.

FIG. 29. T7 protein expression of P. aeruginosa O5 Rol. This autoradiogram shows ³⁵S-labeled proteins expressed by pFV401, which contains the rol gene, and corresponding control plasmid vector pBluescript II SK in E. coli JM109DE3 by use of the T7 expression system. The arrow indicates the putative Rol protein. Molecular size markers are indicated to the left of the figure.

FIG. 32. Features of the initiation regions. Capital letters for bases indicate one of the following sites: potential ribosomal binding sites (RBS), the presumed start codon (also in bold and double underlined), the second codon where it is AAA (the preferred second codon), and components of the sequences TTAA and AAA from +10 to +13 and from −1 to −3 respectively (Gold and Stormo, 1987). The termination codon of the preceding gene is indicated by a bar above if it is in the region shown. The reference sequences involved are also shown above the set of sequences.

FIG. 33. NAD-binding domains of PsbA, PsbK and PsbM aligned with those of other bacterial proteins involved in polysaccharide biosynthesis. The consensus sequence for an NAD-binding domain (Macpherson et al., 1994) is shown at the bottom of the figure in bold underline. The first column contains the protein names; the second column indicates the location of the NAD-binding, site within the protein; the third column shows the alignment of the NAD-binding domains with highly conserved residues indicated in bold type; and the fourth column gives the reference for the protein shown. Most of the proteins in this group of sugar biosynthesis enzymes function as dehydrogenases/dehydratases. Note that PsbM, BpIL, and TrsG have two putative NAD-binding domains, instead of one. The presence of two domains supports the proposal that these large proteins arose from fusion of two smaller proteins.

FIG. 43. Physical map of the 5 end of the wbp cluster. The wzz gene ends approximately 800 bp upstream of wbpA, the first gene of the wbp cluster (8). The probe used to identify a HindIII fragment containing the intact wzz gene for cloning into pFV401 is shown as a black bar above the restriction map. The site of insertion of the gentamicin cassette used to create the wzz knockout mutants is indicated by a black triangle. Key: B, BamHI; H, HindIII; S, SstI; X, XhoI.

FIG. 44. Comparison of hydropathy plots of selected Wzz-like proteins. The hydropathy plots of selected Wzz-like proteins were calculated using PC/GENE SOAP. The X axis represents amino acid residues, while the Y axis represents relative hydropathy. Positive values indicate hydrophobicity; negative values indicate hydrophilicity. A, P. aeruginosa O5 Wzz, U50397; B, E. coli O111 Wzz, Z17241; C, E. coli o349, M87049; D, E. coli FepE, P26266; E, Y. enterocolitica O8 Wzz, U43708; F, Y. pseudotuberculosis Wzz,; G, V. cholerae O139 OtnB, X90547.

FIG. 45. Expression of P. aeruginosa Wzz in vitro. The 40 kDa Wzz protein (indicated by black arrowhead) was expressed from the insert of pFV401 in both orientations. A 28 kDa protein was also expressed in both orientations and may represent either a breakdown product of the 40 kDa polypeptide, or initiation of translation from a secondary ribosome-binding site. There are several smaller ORFs encoded on the positive strand of the 2.3 kb insert of pFV401 which could correspond to the 10 kDa protein.

FIG. 46. Analysis of LPS from wzz knockout mutants. LPS from P. aeruginosa serotypes O5 and O16 and their corresponding wzz mutants was examined. FIG. 46A: Silver-stained 12.5% SDS-PAGE. FIG. 46B: Western immunoblot using MAb 18-19, specific for B-band LPS from the O5 serogroup (serotypes O2, O5, O16, O18, O20). FIG. 46C: Western immunoblot using MAb MF15-4, specific for serotype O5 B-band LPS. The plasmid pFV401-26 contains the O5 wzz gene cloned downstream of the lacZ promoter of shuttle vector pUCP26.

FIG. 47. Ability of P. aeruginosa O5 Wzz to function in E. coli. Panel A. Silver-stained SDS-PAGE gel of E. coli CLM4 containing the Shigella dysenteriae rfb cluster on pSS37, with and without the P. aeruginosa wzz gene in pFV401.

Panel B. Western immunoblot of E. coli HB101 containing the P. aeruginosa O5 wbp cluster in pFV100, with and without the P. aeruginosa wzz gene in pFV401. The membrane was incubated with MAb MF15-4, specific for serotype O5 B-band LPS.

FIG. 48. Western immunoblot analysis of lipopolysaccharide (LPS) isolated using the hot water-phenol method of Westphal and Jann. Lanes O5 are LPS from the parent strain, while lanes F1 and F2 are LPS from two mutants containing a gentamicin cassette inserted at the SstI site within the open reading frame of wbpF. The monoclonal antibodies used are N1F10, specific for A-band LPS, and 18-19, specific for B-band LPS. Note that a knockout mutation of wbpF abrogates both A-band and B-band LPS expression.

We claim:

1. An isolated P. aeruginosa B-band gene cluster containing the following genes:wzz, wbpA, wbpB, wbpC wbpD, wbpE, wzy, wbpF, wbpG, wbpH, wpsI, wbpJ, wbpK, wbpL, wbpM and wbpN involved in the synthesis, and assembly of lipopolysaccharide in P. aeruginosa.

2. An isolated P. aeruginosa B-band gene cluster as claimed in claim 1 wherein the genes are organized as shown in FIG. 1 (SEQ.ID.NO:1).

3. An isolated nucleic acid molecule encoding:
(1) (a) Wzz; (b) WbpA; (c) WbpB; (d) WbpC; (e) WbpD; (f) WbpE; (g) Wzy; (h) WbpF; (i) WbpG; (j) WbpI; (k) WbpJ; (1) WbpK; (m) WbpM; (n) WbpH; and (o) WbpN involved in P. aeruginosa O-antigen synthesis and assembly;
(2) UvrB involved in ultraviolet repair;
(3) HisH or HisF involved in histidine synthesis;
(4) RpsA, a 30S ribosomal subunit protein S1.

4. A nucleic acid molecule comprising nucleic acid sequences encoding two or more of the following proteins (1) (a) Wzz; (b) WbpA; (c) WbpB; (d) WbpC; (e) WbpD; (f) WbpE; (g) Wzy; (h) WbpF; (i) HisH; (j) HisF; (k) WbpG; (l) WbpI; (m) WbpJ; (n) WbpK; (o) WbpM; (p) WbpN; (q) WbpH; (r) WbpL; and (s) RpsA.

5. A recombinant molecule adapted for transformation of a host cell comprising a nucleic acid molecule as claimed in claim 3 and an expression control sequence operatively linked to the DNA segment.

6. A transformant host cell including a recombinant molecule as claimed in claim 5.

7. An isolated protein characterized in that it has part or all of the primary structural confirmation of a protein encoded by a gene of the psb gene cluster as claimed in claim 1.

8. A purified protein having the amino acid sequence as shown in FIG. 3 or SEQ ID NO:2;, FIG. 4 or SEQ ID NO:3; FIG. 5 or SEQ ID NO:4; FIG. 6 or SEQ ID NO:5; FIG. 7 or SEQ ID NO:6; FIG. 8 or SEQ ID NO:7; FIG. 9 or SEQ ID NO:8; FIG. 10 or SEQ ID NO:9; FIG. 11 or SEQ ID NO:10; FIG. 12 or SEQ ID NO:11; FIG. 13 or SEQ ID NO:12; FIG. 14 or SEQ ID NO:13; FIG. 15 or SEQ ID NO:14; FIG. 16 or SEQ ID NO:15; FIG. 17 or SEQ ID NO:16; or, FIG. 18 or SEQ ID NO:17; FIG. 19 or SEQ.ID. No.: 18; or, FIG. 20 or SEQ.ID. No.: 19.

9. A monoclonal or polyclonal antibody specific for an epitope of a purified protein as claimed in claim 8.

10. A method for detecting P. aeruginosa in a sample comprising contacting the sample with a monoclonal or polyclonal antibody as claimed in claim 9 which is capable of being detected after it becomes bound to protein in the sample.

11. A method for detecting the presence of a nucleic acid molecule as claimed in claim 3 in a sample, comprising contacting the sample with a nucleotide probe capable of hybridizing with the nucleic molecule, to form a hybridization product, under conditions which permit the formation of the hybridization product, and assaying for the hybridization product.

12. A method for detecting the presence of a nucleic acid molecule as claimed in claim 3, or a predetermined oligonucleotide fragment thereof in a sample, comprising treating the sample with primers which are capable of amplifying the nucleic acid molecule or the predetermined oligonucleotide fragment thereof in a polymerase chain reaction to form amplified sequences under conditions which permit the formation of amplified sequences, and assaying for amplified sequences.

13. A kit for detecting P. aeruginosa by assaying for a protein involved in O-antigen synthesis or assembly in a sample comprising a monoclonal or polyclonal antibody as claimed in claim 9, reagents required for binding of the antibody to protein in the sample, and directions for its use.

14. A kit for detecting the presence of a nucleic acid molecule as claimed in claim 3 in a sample comprising a nucleotide probe capable of hybridizing with the nucleic acid molecule, reagents required for hybridization of the nucleotide probe with the nucleic acid molecule, and directions for its use.

15. A method for assaying for a substance that affects one or both of P. aeruginosa O-antigen synthesis or assembly comprising mixing a protein as claimed in claims 8 or a nucleic acid molecule as claimed in claim 3 with a test substance which is suspected of affecting P. aeruginosa O-antigen synthesis or assembly, and determining the effect of the substance by comparing to a control.


SEQUENCE LISTING (100 sequences)

SEQ ID NO: 1 - DNA, 24417 bp, Pseudomonas aeruginosa
ctcgagatattgagcagcgcatacagaacttgcggagagaatgccaaggcagacgtgaagatcgtattgttcagctcaaggaggcgttgaaggtcgcaggtgcgctgaaattggaggagcctccactgatcagtgggcaatcctctgaggagctctcggctatcatgaatggaagtctgatgtatatgcgtggcagtaaggcgattatggccgagattcagacattggaggcgcgtagctctgatgatccttttattccggcgttgcgtactcttcaggagcagcagttattgctgagtagcttgcgtgttaattcggagcgggtttctgtttttcgacaagacggtccgatagaaacgccggactcaccagttcgtccaaggagagcgatgattttgatttttgggttgataattggtggtgtgcttggtggttttctggcgttgtgccggatttttttgaagaagtatgctcgttaggaaagagctagttattgaagtggtgatgcgttgcacgtactttggtcgagtaattttgtggagtaggttttcgttgggtggctcgattgctgaggggtgagaacgtttccatgcggtgtttcctcagctctgtctcctgtgccttggctccttgaacgcagaggttaacagttgagctgtggttgtgggtatgtgacgtctgttgcggtggtgtctggttcctggtgtcgggtgtgcgagaagatgccaagttgcctggcaggtcgttacgtgtcgtagccgtattcgaagctcggcaatcgcggggtgatttacaggactgtgcttaatacggcgcaggcttggtcagggtcgagtcgggtcttcgggtgtcaactggatcgtgcgaaaaccggtttcgtggatgctgataagctcggcttgactggcagtccagggcggttaccaggtctgtggaggcgcaaaatgtataggagcctgcgtgagctgggcaggctgaaggcctgctcgaaagcgagttagcattgtggtccggaagggcatgggtggaccagagtgccgttctgcacggcaaaagccaacttgctcggaggttccctagcgcctatgattacgacgcccttcatttttggccattgccgccaggtgctgtggaaagcgacagtatcccttctttatcgatcttgtgaagatgtcgagagtggtcgcagaaaggattcactcgactgacgaatgaatcgtggaagatttaagttcccgttgtgcggtcgcaggcgcgggcaggtaaaattgaggtgagttggaaaatgatagatgttaacacagtggtagagaagttcaaaagccgacaggccttgattggtatcgtgggtctgggttatgtcggtttaccactgatgctgcgatacaacgccattggtttcgatgtcttgggtatcgatatcgatgatgtcaaggttgacaagcttaatgccgggcagtgctatatcgaacatattccgcaagccaaaattgctaaggcccgtgcaagcggtttcgaggctacgaccgatttcagccgtgtcagtgaatgtgatgccctgatcctttgtgtgccgacgccgctgaacaagtatcgcgagccggatatgagctttgtcatcaataccaccgacgcactaaaaccgtatctgcgcgtagggcaggtggtttcgctggaaagtaccacctatccgggaactaccgaggaagagttgttgccacgcgtgcaggagggtggcctcgtggttggccgggacatctacctggtctattctccggagcgtgaagatccgggcaacccgaacttcgagactcgtaccattccgaaagtgatcggtggtcacactcctcagtgtctggaagtcggcattgccctgtatgaacaggccatcgaccgggtcgtgccggtcagttccaccaaggccgccgagatgaccaagctgttggagaacattcatcgcgcggtcaatatcggtctggtcaacgaaatgaagatcgttgctgatcgcatgggtatcgacatctttgaagtggttgatgctgcggcgaccaagccgttcggtttcactccttactacccagggccgggactgggcgggcactgtatcccgatcgatcccttctacctgacttggaaggctcgcgaatacggactgcatacccgcttcatcgaactgtctggtgaggtcaaccaggccatgccggaatacgtactgggcaaactcatggatggcctgaacgaggcaggcagggccctcaagggcagtcgtgtactggtattgggtatcgcttataagaagaatgtcgacgacatgcgcgagtcgccatccgtggaaatcatggagctgatcgaagccaagggtgggatggtcgcctatagcgatccgcatgtgccggtgttcccgaagatgcgtgaacaccacttcgaactgagcagtgagccgctgactgccgaaaacctggctaggttcgacgctgtagtgcttgcgaccgaccatgacaagtttgactatgagctgatcaaggccgaagccaagctagttgttgacagccgtggcaagtaccgctccccggcggcacacatcatcaaggcttgatcacccatcccagcatgtccatccgctcgtgccagaaggccgggcggatccgctcatttccataggacgaaccatgaaaaatttcgctctcatcggtgctgccggctacatcgctcctcgccatatgcgcgccatcaaagacaccggtaactgcctggtttcggcctatgacatcaatgactcggtcggtattattgatagcatctctccccagagcgagttttttaccgagttcgagttctttcttgatcatgcgagcaacctcaagcgcgactctgctaccgcgctggactacgtatcgatctgctcgcccaattacctgcactacccgcatatcgctgcaggtctgcgcttgggttgcgacgtaatctgcgaaaagccgcttgttccaaccccagagatgctcgatcagttggctgttatcgagcgcgaaaccgataagcgcctctacaacattctgcaactgcgtcatcaccaggcgatcatcgcattgaaggacaaggtcgcccgcgaaaaaagtccgcataagtacgaggtcgatctgacttacattacttcccgcggcaactggtatctgaaaagctggaagggagatccacgtaagtcgttcggcgtggctaccaacatcggtgtgcacttctacgacatgctgcacttcatctttggcaagctgcagcgtaatgttgtgcacttcacttccgagtacaagacagctggttatctggagtacgagcaggcccgtgtgcgttggtttctgtccgtggatgctaacgacctgccggagtcggtcaagggcaaaaagccgacctatcgttcgattaccgtcaacggtgaggaaatggagttctctgaaggctttaccgatctacatacaaccagctacgaagaaattctcgctggtcgtggttatggcatcgatgacgctcgtcattgtgtggaaactgtcaataccattcgcagcgccgtcatcgtaccggcctctgataacgaagggcatccgttcgtcgcggcgcttgcgcgttgaggtagaaaaggagtggccgtcctcggtcacctgtttacagcaggtttccgcaggatcattcatcagcatgtcatctagtagctctaaattgctgaacggtatggtcgcggtaagttcaggcagaaacattcggctggatgtccaggggctgcgggctgttgcagttctggctgtgctagcttaccacgccaacagtgcctggctcagggctgggtttgtcggcgttgacgtgttcttcgtcatttccgggtttatcattaccgccttactggtcgagcgcggtgtaaaagttgatctggtagagttttacgcgggccgtatcaaacgtatttttccagcctatttcgtcatgttggcgattgtctgcattgtctcgacaattctgtttctgcctgatgactatgttttttttgaaaaaagtctacagtcatctgtatttttttccagtaatcactatttcgctaattttggtagttactttgctccgagagctgaagagctgccgctgctgcatacttgttcaatagccaacgagatgcagttttatctgttctaccctgtactgttcatgtgcctgccatgtcgatggcgcttgccggtgttcatcctattagctattttgctgttcatttggagtggctattgcgtattcagcggcagccaagatgctcagtacttcgccttgctagctcgtgtacctgagttcatgtcgggagctgttgtcgcattatcattacgtgatcgtgagctacccgccaggcttgcgatacttgcggggttattgggggcggcgttgctggtctgctccttcattatcatcgacaagcagcactttcccggattctggtcgctcctgccatgcctgggagccgctctgctcattgctgcccgacgtggccctgccagcctgctgctggccagcaggcccatggtctggataggtggtatctcctattcgttgtatctgtggcactggccaattctggcattcatccgttactacaccggccaatacgaattgagcttcgtggcgctgttggcatttctcacaggttcgttcctgctggcctggttctcataccgctacatcgagacacctgccagaaaggctgtgggtctgcgccagcaggcgctgaagtggatgttggccgccagtgtggtagctatagtggttacggggggggcgcagttcaatgtgttggttgtggcgccggcgccaattcagttgacgcgctacgctgtaccagagtcgatctgccatggtgttcaggtaggggagtgcaagcgaggcagcgtcaatgccgtaccccgtgtgctggtgatcggtgatagccatgctgcgcagcttaactacttcttcgacgtggttggcaacgagtcaggtgtggcttaccgagtactcaccggaagcagttgtgtgccaatacctgctttcgatcttgaacgtttgccccgttgggcgcggaaaccctgccaagcgcagattgatgcagttgcccaatcaatgttgaactttgacaagatcattgtggcgggcatgtggcagtatcagatgcagagtccggcatttgcccaggctatgcgtgccttccttgtcgataccagctatgccggcaagcaggtcgctctactcgggcagataccgatgttcgaatcaaacgtgcagcgtgtgcgtcgtttcagggagctgggtttgtcagctccgcttgttagctccagctggcaaggtgcgaaccagctgttgcgtgctctagccgagggtattccaaacgtacggttcatggatttttcttccagcgccttcttcgccgatgctccttatcaggacggagagcttatttaccaggatagccatcaccttaacgaggtgggggctcgccgctatggatatttcgcgagccgtcaattgcagcggctgtttgaacaaccacaatcgagtgtgagtctcaagccatgagttattatcagcaccccagcgcgatcgtcgacgacggtgcgcagatcggtagcgactcccgagtttggcacttcgtgcacatctgtgcaggtgcccggattggcgcaggggtttcgttgggtcagaacgtattcgtcggcaacaaggtcgttattggtgatcgctgcaagatccagaacaacgtgtcggtatatgacaatgtcactctcgaagagggcgtgttctgcgggccgagcatggtatttaccaacgtttacaacccccgctcgttgatcgagcgcaaggatcagtaccgtaacacgttggtaaaaaaaggtgccacgcttggtgccaactgcactatcgtctgtggcgtgactattggtgaatatgccttcctgggtgcgggtgcggtcattaacaagaatgttccatcttatgccctgatggtaggcgtgcccgctcgacagattggttggatagcgaattcggtgagcagctgcagctgaacgagcagggcgaagctgtctgctcacactccggtgcgcgctatgtactcaatggaaagatcctgagcaaggtggacgtgtgaccatgattgaattcatcgacctgaagaaccagcaagcgcgtatcaaggacaagatcgatgccggtatccagcgcgtgctgagacacgggcagtacattcttggcccggaagtcactgagcttgaggatcgcctcgccgatttcgtcggcgctaagtactgcatcagttgcgccaacggtactgacgctctacagattgtgcagatggccttgggtgttggcccaggtgacgaagtaatcacccctggttttacttatgttgcgacagcggagaccgtcgcgcttttgggagccaagccggtttacgtggatattgatccacgcacctacaatcttgatccgcagttgctggaggctgcgatcacaccgcgtacgaaggctatcattcctgtttcgctgtatggccagtgtgcagacttcgatgcaatcaacgccattgcctccaaatatggtatccctgtcattgaggatgctgcacagagcttcggtgcttcgtacaagggtaagcgttcttgtaatctgagtaccgttgcctgcaccagcttcttcccgagcaaaccgttgggttgctatggggatggtggagcgatcttcactaacgacgatgaactggctactgctattcgtcaaattgcccggcatggtcaggaccgccgctatcatcacattcgtgtgggggtgaatagtcggttggacacattgcaggctgcgattcttctaccgaagcttgaaattttcgaggaggagattgcgttgcgccagaaggtagccgcggagtatgacctatcactgaaacaggtcggtatcggcacgccgtttattggaagtggataacatcagtgtttatgcccagtatacggtgcgtatggataatcgagagtctgttcaggcttctttgaaagctgccggggttccaactgctgtgcattaccctattccgcttaataagcagcctgctgttgcggatgagaaagcgaaactaccagtgggtgacaaggctgctactcaagtaatgagcctacccatgcatccctatctggatacggcatccatcaaaatcatctgtgctgcgttgacgaattgacggatgtatatacttgctcgagtcgacaggtctattctgctgaacacagtgttactgtttgctttcttttcagcgacagtgtgggtgaataataattatatctatcatctctatgattatatggggtctgcgaaaaaaactgtcgacttcggcttgtatccgtacttgatggtcttggcgctcatctgtgccctgttgtgtggaggggcaattcgcaggccaggtgatctgttagttacattattagttgtaatacttgttcctcattcattggttcttaatggagctaatcaatattctccggatgcgcaaccatgggctggcgtgcctctggcaattgcttttggtattttgatcatcggcattgtcaataagataagattccatccgctaggtgcattgcagcgagaaaaccaaggaaggcgaatgttagtgctactgtcagtactcaacatagtagtgcttgtgtttattttctttaaaagcgctggttatttttcctttgactttgctgggcagtatgctcgccgtgcacttgctcgtgaggtttttgctgcgggttctgcaaacggctacttgtcgtcaatcggtacccaggcattctttcctgtgttgtttgcctggggggtctacagacgacaatggttctacttggtcctgggtattgtcaatgcactagtgctgtggggagcgtttggacagaagtatccttttgtcgtgttgtttctaatttatggcctgatggtttattttcgacgattcggtcaggtcagagtgtcttgggttgtctgcgcactattgatgcttttgcttttaggggcgttggaacatgaggtgtttggctattcattcttgaatgattattttctacgtcgtgcttttattgtgccttccaccctgttgggggcagttgatcagtttgtgtctcagttcggatccaattattacagggataccctgttgggcgcgctcttgggtcagggtaggactgagccgttgagctttcgtctggggacggaaattttcaataatcccgatatgaatgcgaatgtaaacttcttcgcgatagcctatatgcagttgggttatgtgggggttatggctgagtcgatgttggtgggcggtagtgtcgttctcatgaatttcttattttcgaggtatggtgcattcatggccattccggttgctttgttatttactacaaagattcttgagcagcccctgctaactgtaatgcttggctctggtgttttcttgatactgcttttccttgcgctaatttcttttccactcaagatgtctttaggaaaaactctatgagtgcggcttttatcaaccgtgtcgcacgagtattagtaggcaccttgggagcacagctcataacgattggtgtcactctgctactggttcgtctgtattctcctgctgaaatgggcgctttcagtgtttggctatcgttcgctacgatttttgcagttgtagttactgggcgctatgagttggctattttttcgactcgagaagagggcgaactccaggcaatcgtcaagctgatacttcagttgacactattgattttcgttgccgtggcgattgctgttgttataggtagacatctgattgagtcgatgccagttgtgatcggtgaatactggttcgcattggcggtggcttcgctggggttggggataaataagctagtcttgtcgttacttacatttcaacaatcttttaatcggttgggagttgctcgtgtaagcctggctgcatgtattgccgttgcacaagtttcagctgcatatttactggagggcgtatcagggctgatctatggccagctgtttggtgtcgtcgtagccacggcgcttgcggccctttgggtaggaaagtcgctgattttaaattgtatcgagacaccgtggcgtatggtacgacaagtagcggtacagtacatcaatttcccgaagttttctctgcctgcggatctggtcaacacggttgccagtcaggtgcctgtgattttattggcggcaaagtttggtggagacagtgcaggctggtttgccctgactctgaagataatgggagctcccatttccttgttggctgcttcggtgctcgatgtgttcaaagaacaagccgctcgtgactaccgagagtttggtaattgccgaggtatcttcctcaagactttcaggttgcttgccgtcctcgcgctacctccttttattatatttggttcattggcgagtgggcctttgggttagtctttggcgaagcgtgggctgagtcggggcgttatgctgtattgatggttccgttgttttatatgcgtttcgtggtgagtccgctcagctatacaatctatattgcccagcggcagagtatggatttgttgtggcagctagccttgttgctcctgacgtttatctgttttaccttgcctgactctgtcgactcggtgttgtggttttactccatagcatatgctgttatgtattttgtctatttctggatgtccttccagtgtgccaagggagatgccaagtgatcgttgttattgattacggtgtaggtaacattgcttcagtcttgaacatgctgaagcgagttggtgccaaagccaaggcatccgatagccgagaggatatcgagcaggcggagaaactgattttgcctggtgtcggtgcttttgacgccggaatgcaaacactacgcaagagtgggctggtggatgtactgacagagcaggtcatgatcaaacgaaagccggtcatgggggtgtgtctcgggagtcaagatgctggggctgcgatctgaggagggagcggaaccggggcttggatggatcgatatggatagcgtccgtttcgaaaggcgtgacgaccgaaaggttccacatatgggctggaatcaagtgtccccgcaattggagcatcctatacttagcggtataaacgagcaaagccgattctattttgttcatagttattatatggttccgaaagacccagacgatatcctgttgagttgtaattatggacaaaaattcactgcggcggtggctcgggataatgttttcggatttcagtttcatcctgagaagagtcataaattcggtatgcagttattcaaaaacttcgtggagcttgtctgatggtccggaggcgcgttatcccatgcttgctgctcaaggatcgcggtctagtgaaaaccgtgaagttcaaggagcccaagtacgttggagacccgatcaacgcaatacgcatcttcaatgagaaagaagtcgacgaactgattttgctggatatagatgcttccaggctcaatcaagagcctaactatgagttgatcgcggaagtggctggtgagtgttttatgcctatttgctatgggggcggtatcaagacattggagcatgcggaaaaaatcttttccctaggtgtcgaaaaagtttcgataaataccgccgctcttatggatctttcgttgattcgaagaattgccgataagtttggttcgcaaagcgtagttggctctatcgactgccgcaagggtttctggggaggacactccgtgttctcagagaatgggacgcgcgacatgaaacgctccccattggagtgggcgcaagcgctcgaagaggctggagtgggtgagatttttctaaattctattgatcgagatggagtgcagaaaggcttcgacaacgctctagtggaaaatatcgcttctaacgtccatgtgccagtgatcgcctgtggtggagctggctccatcgctgacctcatcgatctttttgagcgtacgtgtgtgtcggcagtagcggcgggaagcctattcgttttccatggcaagcatcgtgcggtactgattagttatccggatgtcaacaagctcgacgtcggttagagtgagctgagttatttatggcaaggacgcttgttggcaacgctatatgcgcttcaagattgtcgaactaaatttgagtttgtcagtggggcgttccattaggcaggccgaggtgagtgcttcgggaggttgttgtgatgaagatctgttcgcgctgtgttatggatacatctgacgctgaaatcgtatttgatgaggcgggagtctgtaatcactgccataaatttgacaatgttcagtcccggcagctgttttccgatgctagtggtgagcagcgccttcaaaagataattgggcagatcaagaaggacggttcaggtaaggattatgactgcatcattggccttagtggcggcgtagatagttcctatcttgctgtaaaggtcaaggatcttggcttgcgcccactggttgtgcatgtggacgccggctggaatagcgaacttgcagtcagtaatattgaaaagattgtaaaatattgcggttttgatttacatactcatgtaataaactgggaggaaattcgtgatcttcagttggcttatatgaaagctgctgtcgccaatcaggatgtgcctcaagatcatgccttcttcgctagtatgtatcactttgctgtgaagaataatattaagtacattctgagtggtggtaatttggccactgaggcagtattcccagatacatggcacggcagcgctatggatgcaataaacctaaaggctattcacaaaaaatatggtgagcgtccgctaagggactacaagactattagttttcttgagtactatttctggtatccctttgtcaaaggaatgagaacggtccgtccgttgaatttcatggcctatgataaggccaaggctgaaaccttccttcaagaaacgataggctatcgttcttacgcgcgaaagcatggagagtcgattttcaccaagcttttccagaactactatctaccgaccaagtttggctatgataaacgcaaactgcactactccagcatgattttgtctgggcaaatgacgcgtgacgaagctcaggctaaactggctgagccgctatatgatgcagatgaactgcagtttgatatcgaatatttctgcaagaagatgcgaatcacccaggctcaatttgaagagttgatgaatgcacctgttcatgactattcggagtttgccaactgggattctcgacagaggattgcgaaaaaagttcaaatgattgtccagcgtgcgctgggtcgtcgcatcaatgtctactcgtgatgaccggggccgctcatgactaaagttgctcatttgacatcggttcactcgcgttatgatattcgtatatttcgaaagcagtgtagaacactctctcaatacggatacgatgtgtatctggttgtcgcagatggtaagggtgatgaagtcaaggatggtgtaaggattgttgatgtcggagtactctcaggtcgcttgaatcgtattctaaaaaccacccgaaaaatttatgaacaggctttggcgcttggggctgatgtctatcattttcatgatcccgaactgatacctgttggtcttcgactgaaaaagcaaggtaagcaggttatcttcgactcccatgaggatgtgccgaagcaactgctgagtaaaccttacatgcgaccgtttttacgccgtgtagtggctgtgttattttcctgctatgagaaatatgcatgccctaagctggatgcagtccttacggcaacgccgcatattcgtgaaaaatttaaaaatattaatgggaatgttctagatattaataactttcccatgttgggtgagttggatgcgatggttccttgggcaagcaagaaaactgaagtctgctacgtcggtggtatcacttccattcgtggtgttcgtgaagtcgttaagagtcttgagtgcttgaagtcctcggcgcgcttgaatttagtgggaaagttttcagagccagagatagaaaaagaagtcagagcgctcaagggatggaactccgttaacgaacatggtcagcttgatcgagaagatgttcgtcgtgtactcggtgactctgttgccgggttggtgacatttctcccaatgcctaatcatgttgatgcacaacctaataagatgttcgagtatatgtcgtcgggaatccctgtgatcgcttccaattttcctctctggcgggaaattgttgaaggtagcaattgtggtatatgcgtagatcctctaagtcctgctgccattgctgaagcgatcgactatctggtaagtaatccgtgtgaggcggcagcgctgggacgtaatggccagcgggcagtgaacgaacgttataactgggatttggaagggcgcaaactagcgcggttctattccgatctactgagtaagcgagattccatatgaaaattctgaccatcattggtgcgcgtccgcagtttattaaagcgagtgtggtttcaaaggctatcattgagcagcagaccctttcggaaatcatcgttcatactggtcagcattttgatgccaatatgtctgaaatatttttcgaacagctgggtattccaaagccggattaccagttggatatccatggtggtactcacggccaaatgaccgggcgtatgctaatggagatcgaggatgtaattctcaaggagaaacctcatcgcgtattggtatacggcgataccaactctaccttggctggagcgttggctgcctccaagctgcatgttcctatcgcacacatcgaagccggcctgcgaagtttcaatatgcggatgccggaggaaattaaccgtattcttactgatcaggttagtgatattctgttttgccctactcgagttgcaattgataatctcaagaatgaaggtttcgaaagaaaggctgcgaagatagtcaacgtgggtgatgtgatgcaggatagcgctctattctttgcgcagcgtgcaacctcgccaattggacttgcgtcacaagatgggtttattctcgcgaccctgcatcgtgccgagaacaccgacgatccagttcgcctgacttcgatagtcgaggctctgaatgaaatccagattaatgttgcacctgtggtgctacccctgcatccacgtacccgcggtgtcatcgagcgcctagggctcaagctggaagtgcaggttatcgatcctgtcggatatctggaaatgatctggctgttgcaacgctctggcctggtgctcacggacagcggcggtgttcagaaagaagcattcttcttcggcaagccctgcgtgaccatgcgtgaccagaccgaatgggtggagctagtgacctgtggagccaacgttcttgtgggagcggcccgcgacatgattgtcgaatctgcacggactagcctgggaaagaccattcaagacgatggtcagctttacggaggcggtcaagcctctctcggattgctgaatatcttgccaagctgtgatgctttgcgtgtcgagtttaaataaaggatttatttagttccatgaacgtctggtatgtgcatccctatgctggcggccccggagttggtcgttattggcggccttattatttctccaagttttggaatcaggctgggcatcggtcggtcataatctcggcaggctatcaccatctgctggaaccggatgaaaagcgttcgggcgtcacctgtgtaaatggagccgaatacgcatatgtacctactttgcgctatttgggcaatggcgtgggcagaatgctatcgatgctcatatttaccatgatgttgctgccattctgcctgatcttggccctgaagcgtggaacgccggatgcgattatctactcatcgcctcacccgtttggcgtcgttagctgttggctggctgctcgcctgctaggtgcgaaatttgtatttgaggtgcgcgatatctggcctttgagtctggtcgaactgggaggcttgaaagctgacaatcccctggtgcgtgttaccggttggatcgaaagattctcctatgcgcgagctgataagatcatcagtctgctgccatgtgcggagccgcacatggccgacaaaggacttcccgctggaaagttcctgtgggttccgaatggcgttgacagcagcgatatctctcctgatagcgctgtgagttcaagtgatttggtccggcatgtacaagttctcaaggagcagggtgttttcgttgtgatctatgctggagcgcacggcgaacccaatgctctggagggattggttcgctctgccggactgctgcgcgagcgtggtgcaagtatcagaatcattctggtgggcaagggagagtgcaaagagcaactcaaggcgattgccgcacaggatgccagcgggctagtggagtttttcgatcagcagcccaaagagactatcatggctgtcctgaagctggcgtcggcgggctacatctcgctcaagtcagaaccgatcttccgctttggcgtgagccccaacaagctatgggattacatgctggttgggttgccagtcattttcgcctgcaaggcagggaacgacccggttagtgactacgattgcggtgtatctgccgacccagatgcccctgaggatattactgcagccatcttccgtctgttgctgctgagcgaagacgagcgtcgcacaatggggcaaagagggcgtgatgcggtcctggagcattatacctacgagagtctggctcttcaggtgttgaacgcccttgctgatgggcgcgcagcatgaaagctgtcatggtgaccggtgcatcaggattcgtcggatcggccttgtgctgtgagcttgctcggacagggtatgcggtgattgcggtggtacggcgggttgttgaaagaataccttctgtgacgtacatcgaagctgatctgaccgatccagccacgtttgccggcgagttcccgacggtggattgcattattcatctcgctggacgtgcccatatactcactgacaaggttgcagacccgctcgccgcatttcgtgaagtcaaccgagatgcgactgtccggttggctacccgtgcgctcgaggctggggtgaagcgtttcgtgtttgtcagttcaattggcgttaacggtaacagcacccggcaacaggctttcaacgaagattctccagccggcccacatgcgccctatgccatctccaaatacgaggctgagcaggagctggggactttgctccggggtaaaggtatggagttggtggttgtccgaccgcctttgatctatgccaatgatgcgccaggtaacttcggccgtttgctcaagctcgtcgctagtggtctgccgcttccgcttgacggtgtccgtaatgcgcgcagcctggtttctaggagaaacatcgtgggtttcctgagtctttgtgccgaacaccccgatgctgcgggcgaactgtttctggtggcggatggcgaggatgtttccattgcgcaaatgatcgaggccctgagtcggggaatgggcaggcgtccagctcttttcacgtttccagcggtgctgctgaagcttgtaatgtgcttgctgggtaaggcttccatgcatgaacagctctgtggctcgttacaggtcgatgcttccaaggcccgccggctgctcggctgggttcccgtcgagactattggtgccggtctgcaagcagcaggtcgagagtacattcttcgccagagggagcgccgaaaatgacggacacatccaaacccctggtcggcaattacgctgaactttaataagttctctttccaatgatgatctggatgatcgcgtgtctagttgtcttgctgttttcatttgtcgctacctgggggctgcgtcgctatgcattagcgacgaaactgatggatgttccgaatgcccgtagctcccacagtcaaccgacgcctagggggggaggtgttgcaatcgttctggtcttccttgcagcgttggtgtggatgctgagtgcaggcagtatctccggcggctgggggggggcgatgctgggtgcaggttctggcgtggcactgttagggttcctggatgaccatgggcacattgctgcgcgttggcggctgctcggccatttctcagcagcgatatggatcttgctgtggacgggtggtttcccgccgctggatgtggttgggcatgctgtcgacttaggatggctgggccacgtattggcagttttctatttggtatgggtgctgaacctttataacttcatggatggcattgatggtattgccagtgtcgaggccattggtgtctgtgtaggaggggccctgatctactggcttacagggcatgtcgcgatggttggtatccctctgttgctggcgtgcgcggtcgccggcttcctgatctggaacttccctccagctcgaatcttcatgggtgatgcggggagtggttttcttggtatggttattggtgcactagctattcaggctgcatggaccgccccctcgctgttctggtgctggttgatattgctgggagtgttcatcgttgatgcaacctatactctgatccgccggatcgccagaggggagaaattctatgaggcgcatcgcagccacgcttatcagtttgcctcgcgtcgttatgctagccatctgcgggttaccttgggtgttctggctatcaacactctttggttgttgcgttggcactgatggttgcattgggttggatcagcggcttcatcggtatcctggttgcttatgctcctctttgcctcttggcggtaggatacaaggcgggttccttggaaaaatcctaagccgtggattgacctgctccccgatttcagtaccacgccgaacttagtagagtctgttttccgagcaggagacggcagtgaaaaagcgttttactgaagaacagattctagactttctgaagcaggcagaagccggtgtgccggtgaaggagctgtgtcgccgacacagcttcagtgatgccacgttctacacctagcgggccaagttcgtcggcatgaccgtgccggatgccaagcgcctgaaggatctcgaactggaaaacagccggctgaagaagttgctcgccgagtccctcctcgacatcggggcgctgaaagtggtcacccggggaaagggggagcccggcagcggggcgggggggcaggagattcaggcgcaaaccgacatctccgagcgtcgtgccctgtcagttgttcaggctgtcccgctctgtgttgtgccaccagccgcgaactagtgtgcaaaacaccgagctgcaagcccaactggtggaactggcaagggcttcggcactttggctatcaccgcctgcacattctgctgcggcgtgctggtgtgcagatcaactacaagcggacttaccggctatactgagccgtcggcttgatggtgaagcggcggaggcgccgccacaggggcgcggtggcgtgcgaatgcctgagcctgccgagcgcaccgaactaggtcttgtcgatggatttcgtcttcgacgcgctcagcactgggcgacggatcaaatgcctgacggtggtcgatgacttcaccaaggagtcggttggcatcctggtggagcacggtatcagcggttttcgtgtcacacgggcgctggacagatggcacggttgcgcggttacccgaaggcgatccgcacccccgagttcaccggcaaggcgcttgatcagtgggcctatcggcgtgatattaagttgaagctgactcagtccggcaagcccacgcagaacgccttcatcgtcattccaacggcaagttccgcaatgagcactgctgctcgctggtcgaagccagaatccgcatcgtggcctggcggcacgattacaacgagcaccgaccgtccagcgccattggcaatctcacctcgctagagtttgctgcaagttggcgaactcgccagcagcaactgaagcaggaaaattgatgtcaaccccagggcctactacctaggcagcgtactaaaactgggggcaggtcatctacgatccttgtgataggtatcgacggtgctgtggcgatccgtgcatgtggaactgatctgggattttccctgcgtgtgttttcaggggcctggcagtgattttttgagcattgccatgggggggcgggtttttgcatcctgctcggacgctggctgattcccactcgacgtgctcgtgttcgatgtcacttttactttgctgctgcatcgtttgttatgaggcgataaaattcggcagagctatcgagtcacgcatgatggcacgttggtgtcgtgctgaagtggcatttgccggttatcctttgtggctgtgatcagtttcttctggttattaccctagcattgctggtagtactaagcattatcgacggagtacttgggggcttatcgcgtatgctcctatggcttggatggcgacgagtcttgggaggggatgtcctgagacgtagcgtgggccttgccatattgttgccatggttatctgtctgatctgtctggttggtatggatgtattgaacggggctgataaataggatgttggataatttgaggataaagctcctgggattgccgcgccgctataagcgaatgctgcaagtcgctgccgatgtgactcttgtgtggctatccctctggctggctttcttggtcaggttgggcacagaagacatgatcagcccgtttagcggccatgcctggctgttcatcgccgccccgttggtggccattcccctgttcatccgcttcggcatgtaccgggcggtgatgcgctacctgggcaacgacgcccttatcgcgatcgccaaggccgtcaccatttccgcgctggtcctgtcgttgctggtctactggtaccgctccccgccggcggtggtgccgcgttccctggtgttcaactactggtggttgagcatgctgctgatcggcggcttgcgtctggccatgcgccagtatttcatgggagactggtactctgctgtgcagtcggtaccatttctcaaccgccaggatggcctgcccagggtggctatctatggcgcgggggcggccgccaaccagttggttgcggcattgcgtctcggtcgggcgatgcgtccggtggcgttcatcgatgatgacaagcagatcgccaaccgggtcatcgccggtctgcgggtctataccgccaagcatatccgccagatgatcgacgagacgggcgcgcaggaggttctcctggcgattccttccgccactcgggcccggcgccgagagattctcgagtccctggagccgttcccgctgcacgtgcgcagcatgcccggcttcatggacctgaccagcggccgggtcaaggtggacgacctgcaggaggtggacatcgctgacctgctggggcgcgacagcgtcgcaccgcgcaaggagctgctggaacgttgcatccgcggtcaggtggtgatggtgaccggggcgggcggctctatcggttcggaactctgtcggcagatcatgagttgttcgcctagcgtgctgatcctgttcgagcacagcgaatacaacctctatagcatccatcaggaactggagcgtcggatcaagcgcgagtcgctttcggtgaacctgttgccgatcctcggttcggtgcgcaatcccgagcgcctggtggacgtgatgcgtacctggaaggtcaataccgtctaccatgcggcggcctacaagcatgtgccgatcgtcgagcacaacatcgccgagggcgttctcaacaacgtgataggcaccttgcatgcggtgcaggccgcggtgcaggtcggcgtgcagaacttcgtgctgatttccaccgacaaggcggtgcgaccgaccaatgtgatgggcagcaccaagcgcctggcggagatggtccttcaggcgctcagcaacgaatcggcaccgttgctgttcggcgatcggaaggacgtgcatcacgtcaacaagacccgtttcacaatggtccgcttcggcaacgtcctcggttcgtccggttcggtcattccgctgttccgcgagcagatcaagcgcggcggcccggtgacggtcacccacccgagcatcacccgttacttcatgaccattcccgaggcagcgcagttggtcatccaggccggttcgatggggcagggcggagatgtattcgtgctggacatggggccgccggtgaagatcctggagctcgccgagaagatgatccacctgtccggcctgagcgtgcgttccgagcgttcgccccatggtgacatcgccatcgagttcagtggcctgcgtcctggcgagaagctctacgaagagctgctgatcggtgacaacgtgaatcccaccgaccatccgatgatcatgcgggccaacgaggaacacctgagctgggaggccttcaaggtcgtgctggagcagttgctggccgccgtggagaaggacgactactcgcgggttcgccagttgctgcgggaaaccgtcagcggctatgcgcctgacggtgaaatcgtcgactggatctatcgccagaggcggcgagaaccctgagtcatcgttctccggaaaaggccgcctagcggccttttttgttttctccgtacgatgtttccggtgccggaccaggaagcgactgctttgctggggctgtcgatccaggtgcgttccacggcgataaggtggtttcgtggatgggcatgaagccctctacgtggtcattcatctctgaaggagtgcacccatgcacctaatcaaatccgctctgcttctcatcctgttcgcctgtcttccgttttcggcttccgccgcaccggtcgccgtcgccaagaatccgctggccgcaacgacacctgcgacgaccgtgtcgccgggggagcaggtcaatatcaatacggtcgacgaggccgccctgatacgggggctcaacggtgtcggcgaggccaaggccagggcgatcctcgagtatcgtgcggcccatggtccgttcgtctcggtggatcaactgctggaagtgaaaggggtaggcccggcgttgctggagaagaaccgggcgcggatcgtcatcgagtgaggtgcgactgaaggggcgaactttcgtcccgataacgaaaaagcccccggcatgtgccgagggctttgaatttggctccgcgacctggactcgaaccagggacccaatgattaacagtcatttgctctaccgactgagctatcgcggaacagcgaggcgtatgttactgattaaaaaggggaagcctctcccgatgacttccccattttccctacaggacctggacgatggccttggtgatggtctccaggttcgatttgttcagcgcggcgacgcagatacggccggtgctgacggcgtagataccgaactcggtcttcaggcgctcgacctggtcggcggtcaggccggaataggagaacatgccacgttggcgaccgacgaaactgaagtcgcgcttggcgccgtgggctgccagttgctcgaccatcgccaggcgcatgtcgcggatgcggtcgcgcatctcgcccagttcctgctcccagagggcccgcagttccgggctgttgagcacggaggagacgacgctggcgccgtgggtcggtgggttcgaatagttggtgcggatcacccgcttcacctgggacagcacgcgggccgattcatcgcggctttcggtcacgatcgagagggcgccgacgcgttcgccatagagcgagaaggatttggagaacgagctggaaacgaagaagctcaggcccgactgggcgaacaggcgcaccgcggcggcgtcttcctcgatgccgttgccgaagccctggtaggcgatgtcgaggaacggcacgtggcccttggccttgagcacgtccagcacctgtttccagtcgtccagctcgagatcgacgccggtcggattatggcagcaggcgtgcagaaccacgatcgagcgggccggcagggcattcaggtcttccagcaggccggcgcggttcacgccattgctggcggcgtcgtaatagcggtagttctgcaccgggaagccggcggcttcgaacagtgcgcggtggttttcccagctcgggtcgctgatggccacggtggcgtcgggcagcaggcgcttgaggaagtcggcgccgagcttgagcgcgccggtgccgccgacggcctgggtcgtgaccacacggccggcggccagcagctcggactcgttaccgaacagcagtttctgtacgccctggtcgtaggcggcgatcccttcgatcggcaggtagccgcgcggcgcgtgggcctcgatgcgggccttctcggcagcctgcacggcacgcaacagcggaatgcgcccctcctcgttgtagtacacgcccacgcccaggttgatcttgcccggacgggtatcggcgttgaaggcttcgttcaggccaaggatgggatcacgcggtgccatttcgacggcagaaaacagactcattttgcggctgctcggagtgtgaagagaggagggcaacgcaacccgttatgcgggggcgcaaagggttgcgcaaacggggggttattatagacaccccttgatgcatgcggcgacatttaggtgcatgctttcagctatttctgacgccggattttccttggcgtcacagctccctgcgaggtttttcatggatacgttccaactcgactcgcgcttcaagcccgccggcgaccagccggaagccatccggcaaatggtcgaggggctggaggcggggctttcgcaccagaccctgctgggggtgacgggctctggcaagactttcagcatcgccaacgtgattgcccaggtgcagcgcccgaccctggtcctggcgccgaacaagaccctggcggcccagctctacggggagttcaagacgttcttcccgcacaattccgtggagtacttcgtttcctactacgactactaccagccggaggcctacgtcccgtcttccgatacctatatcgagaaggactcctcgatcaacgaccatatcgagcagatgcgcctgtcggcgaccaaggcgctgctcgagcgtccggatgcgatcatcgtcgccaccgtgtcgtccatctacggcctcggtgatcccgcgtcctacctgaagatggtcctgcacctggaccgcggcgaccgcatcgaccagcgcgaactgctgcggcgactgaccagcctgcagtacacccgcaacgacatggatttcgcccgtgcgactttccgtgtgcgtggcgatgtgatcgacatcttcccggccgaatccgatctcgag

SEQ ID NO: 2 - protein, 158 aa, Pseudomonas aeruginosa
RDIEQRIQNLRRECQGRREDRIVQLKEALKVAGALKLEEPPLISGQSSEELSAIMNGSLMYMRGSKAIMAEIQTLEARSSDDPFIPALRTLQEQQLLLSSLRVNSERVSVFRQDGPIETPDSPVRPRRAMILIFGLIIGGVLGGFLALCRIFLKKYAR

SEQ ID NO: 3 - protein, 436 aa, Pseudomonas aeruginosa
MIDVNTVVEKFKSRQALIGIVGLGYVGLPLMLRYNAIGFDVLGIDIDDVKVDKLNAGQCYIEHIPQAKIAKARASGFEATTDFSRVSECDALILCVPTPLNKYREPDMSFVINTTDALKPYLRVGQVVSLESTTYPGTTEEELLPRVQEGGLVVGRDIYLVYSPEREDPGNPNFETRTIPKVIGGHTPQCLEVGIALYEQAIDRVVPVSSTKAAEMTKLLENIHRAVNIGLVNEMKIVADRMGIDIFEVVDAAATKPFGFTPYYPGPGLGGHCIPIDPFYLTWKAREYGLHTRFIELSGEVNQAMPEYVLGKLMDGLNEAGRALKGSRVLVLGIAYKKNVDDMRESPSVEIMELIEAKGGMVAYSDPHVPVFPKMREHHFELSSEPLTAENLARFDAVVLATDHDKFDYELIKAEAKLVVDSRGKYRSPAAHIIKA

SEQ ID NO: 4 - protein, 316 aa, Pseudomonas aeruginosa
MKNFALIGAAGYIAPRHMRAIKDTGNCLVSAYDINDSVGIIDSISPQSEFFTEFEFFLDHASNLKRDSATALDYVSICSPNYLHYPHIAAGLRLGCDVICEKPLVPTPEMLDQLAVIERETDKRLYNILQLRHHQAIIALKDKVAREKSPHKYEVDLTYITSRGNWYLKSWKGDPRKSFGVATNIGVHFYDMLHFIFGKLQRNVVHFTSEYKTAGYLEYEQARVRWFLSVDANDLPESVKGKKPTYRSITVNGEEMEFSEGFTDLHTTSYEEILAGRGYGIDDARHCVETVNTIRSAVIVPASDNEGHPFVAALAR

SEQ ID NO: 5 - protein, 766 aa, Pseudomonas aeruginosa
MLCTSLPSTRQLVIWSTSRPVCVGFCPWMLTTCRSRSRAKSRPIVRLPSTVRKWSSLKALPIYIQPATKKFSLVVVMASMTLVIVWKLSIPFAAPSSYRPLITKGIRSSRRLRVEVEKEWPSSVTCLQQVSAGSFISMSSSSSKLLNGMVAVSSGRNIRLDVQGLRAVAVLAVLAYHANSAWLRAGFVGVDVFFVISGFIITALLVERGVKVDLVEFYAGRIKRIFPAYFVMLAIVCIVSTILFLPDDYVFFEKSLQSSVFFSSNHYFANFGSYFAPRAEELPLLHTCSIANEMQFYLFYPVLFMCLPCRWRLPVFILLAILLFIWSGYCVFSGSQDAQYFALLARVPEFMSGAVVALSLRDRELPARLAILAGLLGAALLVCSFIIIDKQHFPGFWSLLPCLGAALLIAARRGPASLLLASRPMVWIGGISYSLYLWHWPILAFIRYYTGQYELSFVALLAFLTGSFLLAWFSYRYIETPARKAVGLRQQALKWMLAASVVAIVVTGGAQFNVLVVAPAPIQLTRYAVPESICHGVQVGECKRGSVNAVPRVLVIGDSHAAQLNYFFDVVGNESGVAYRVLTGSSCVPIPAFDLERLPRWARKPCQAQIDAVAQSMLNFDKIIVAGMWQYQMQSPAFAQAMRAFLVDTSYAGKQVALLGQIPMFESNVQRVRRFRELGLSAPLVSSSWQGANQLLRALAEGIPNVRFMDFSSSAFFADAPYQDGELIYQDSHHLNEVGARRYGYFASRQLQRLFEQPQSSVSLKP

SEQ ID NO: 6 - protein, 160 aa, Pseudomonas aeruginosa
MSYYQHPSAIVDDGAQIGSDSRVWHFVHICAGARIGAGVSLGQNVFVGNKVVIGDRCKIQNNVSVYDNVTLEEGVFCGPSMVFTNVYNPRSLIERKDQYRNTLVKKGATLGANCTIVCGVTIGEYAFLGAGAVINKNVPSYALMVGVPARQIGWIANSVS

SEQ ID NO: 7 - protein, 276 aa, Pseudomonas aeruginosa
MIEFIDLKNQQARIKDKIDAGIQRVLRHGQYILGPEVTELEDRLADFVGAKYCISCANGTDALQIVQMALGVGPGDEVITPGFTYVATAETVALLGAKPVYVDIDPRTYNLDPQLLEAAITPRTKAIIPVSLYGQCADFDAINAIASKYGIPVIEDAAQSFGASYKGKRSCNLSTVACTSFFPSKPLGCYGDGGAIFTNDDELATAIRQIARHGQDRRYHHIRVGVNSRLDTLQAAILLPKLEIFEEEIALRQKVAAEYDLSLKQVGIGTPFIGSG

SEQ ID NO: 8 - protein, 438 aa, Pseudomonas aeruginosa
MYILARVDRSILLNTVLLFAFFSATVWVNNNYIYHLYDYMGSAKKTVDFGLYPYLMVLALICALLCGGAIRRPGDLLVTLLVVILVPHSLVLNGANQYSPDAQPWAGVPLAIAFGILIIGIVNKIRFHPLGALQRENQGRRMLVLLSVLNIVVLVFIFFKSAGYFSFDFAGQYARRALAREVFAAGSANGYLSSIGTQAFFPVLFAWGVYRRQWFYLVLGIVNALVLWGAFGQKYPFVVLFLIYGLMVYFRRFGQVRVSWVVCALLMLLLLGALEHEVFGYSFLNDYFLRRAFIVPSTLLGAVDQFVSQFGSNYYRDTLLGALLGQGRTEPLSFRLGTEIFNNPDMNANVNFFAIAYMQLGYVGVMAESMLVGGSVVLMNFLFSRYGAFMAIPVALLFTTKILEQPLLTVMLGSGVFLILLFLALISFPLKMSLGKTL

SEQ ID NO: 9 - protein, 316 aa, Pseudomonas aeruginosa
MSAAFINRVARVLVGTLGAQLITIGVTLLLVRLYSPAEMGAFSVWLSFATIFAVVVTGRYELAIFSTREEGELQAIVKLILQLTLLIFVAVAIAVVIGRHLIESMPVVIGEYWFALAVASLGLGINKLVLSLLTFQQSFNRLGVARVSLAACIAVAQVSAAYLLEGVSGLIYGQLFGVVVATALAALWVGKSLILNCIETPWRMVRQVAVQYINFPKFSLPADLVNTVASQVPVILLAAKFGGDSAGWFALTLKIMGAPISLLAASVLDVFKEQAARDYREFGNCRGIFLKTFRLLAVLALPPFIIFGSLASGPLG

SEQ ID NO: 10 - protein, 118 aa, Pseudomonas aeruginosa
MLGLRSEEGAEPGLGWIDMDSVRFERRDDRKVPHMGWNQVSPQLEHPILSGINEQSRFYFVHSYYMVPKDPDDILLSCNYGQKFTAAVARDNVFGFQFHPEKSHKFGMQLFKNFVELV

SEQ ID NO: 11 - protein, 251 aa, Pseudomonas aeruginosa
MVRRRVIPCLLLKDRGLVKTVKFKEPKYVGDPINAIRIFNEKEVDELILLDIDASRLNQEPNYELIAEVAGECFMPICYGGGIKTLEHAEKIFSLGVEKVSINTAALMDLSLIRRIADKFGSQSVVGSIDCRKGFWGGHSVFSENGTRDMKRSPLEWAQALEEAGVGEIFLNSIDRDGVQKGFDNALVENIASNVHVPVIACGGAGSIADLIDLFERTCVSAVAAGSLFVFHGKHRAVLISYPDVNKLDVG

SEQ ID NO: 12 - protein, 376 aa, Pseudomonas aeruginosa
MKICSRCVMDTSDAEIVFDEAGVCNHCHKFDNVQSRQLFSDASGEQRLQKIIGQIKKDGSGKDYDCIIGLSGGVDSSYLAVKVKDLGLRPLVVHVDAGWNSELAVSNIEKIVKYCGFDLHTHVINWEEIRDLQLAYMKAAVANQDVPQDHAFFASMYHFAVKNNIKYILSGGNLATEAVFPDTWHGSAMDAINLKAIHKKYGERPLRDYKTISFLEYYFWYPFVKGMRTVRPLNFMAYDKAKAETFLQETIGYRSYARKHGESIFTKLFQNYYLPTKFGYDKRKLHYSSMILSGQMTRDEAQAKLAEPLYDADELQFDIEYFCKKMRITQAQFEELMNAPVHDYSEFANWDSRQRIAKKVQMIVQRALGRRINVYS

SEQ ID NO: 13 - protein, 373 aa, Pseudomonas aeruginosa
MTKVAHLTSVHSRYDIRIFRKQCRTLSQYGYDVYLVVADGKGDEVKDGVRIVDVGVLSGRLNRILKTTRKIYEQALALGADVYHFHDPELIPVGLRLKKQGKQVIFDSHEDVPKQLLSKPYMRPFLRRVVAVLFSCYEKYACPKLDAVLTATPHIREKFKNINGNVLDINNFPMLGELDAMVPWASKKTEVCYVGGITSIRGVREVVKSLECLKSSARLNLVGKFSEPEIEKEVRALKGWNSVNEHGQLDREDVRRVLGDSVAGLVTFLPMPNHVDAQPNKMFEYMSSGIPVIASNFPLWREIVEGSNCGICVDPLSPAAIAEAIDYLVSNPCEAAALGRNGQRAVNERYNWDLEGRKLARFYSDLLSKRDSI

SEQ ID NO: 14 - protein, 362 aa, Pseudomonas aeruginosa
MKILTIIGARPQFIKASVVSKAIIEQQTLSEIIVHTGQHFDANMSEIFFEQLGIPKPDYQLDIHGGTHGQMTGRMLMEIEDVILKEKPHRVLVYGDTNSTLAGALAASKLHVPIAHIEAGLRSFNMRMPEEINRILTDQVSDILFCPTRVAIDNLKNEGFERKAAKIVNVGDVMQDSALFFAQRATSPIGLASQDGFILATLHRAENTDDPVRLTSIVEALNEIQINVAPVVLPLHPRTRGVIERLGLKLEVQVIDPVGYLEMIWLLQRSGLVLTDSGGVQKEAFFFGKPCVTMRDQTEWVELVTCGANVLVGAARDMIVESARTSLGKTIQDDGQLYGGGQASLGLLNILPSCDALRVEFK

SEQ ID NO: 15 - protein, 413 aa, Pseudomonas aeruginosa
MNVWYVHPYAGGPGVGRYWRPYYFSKFWNQAGHRSVIISAGYHHLLEPDEKRSGVTCVNGAEYAYVPTLRYLGNGVGRMLSMLIFTMMLLPFCLILALKRGTPDAIIYSSPHPFGVVSCWLAARLLGAKFVFEVRDIWPLSLVELGGLKADNPLVRVTGWIERFSYARADKIISLLPCAEPHMADKGLPAGKFLWVPNGVDSSDISPDSAVSSSDLVRHVQVLKEQGVFVVIYAGAHGEPNALEGLVRSAGLLRERGASIRIILVGKGECKEQLKAIAAQDASGLVEFFDQQPKETIMAVLKLASAGYISLKSEPIFRFGVSPNKLWDYMLVGLPVIFACKAGNDPVSDYDCGVSADPDAPEDITAAIFRLLLLSEDERRTMGQRGRDAVLEHYTYESLALQVLNALADGRAA

SEQ ID NO: 16 - protein, 320 aa, Pseudomonas aeruginosa
MKAVMVTGASGFVGSALCCELARTGYAVIAVVRRVVERIPSVTYIEADLTDPATFAGEFPTVDCIIHLAGRAHILTDKVADPLAAFREVNRDATVRLATRALEAGVKRFVFVSSIGVNGNSTRQQAFNEDSPAGPHAPYAISKYEAEQELGTLLRGKGMELVVVRPPLIYANDAPGNFGRLLKLVASGLPLPLDGVRNARSLVSRRNIVGFLSLCAEHPDAAGELFLVADGEDVSIAQMIEALSRGMGRRPALFTFPAVLLKLVMCLLGKASMHEQLCGSLQVDASKARRLLGWVPVETIGAGLQAAGREYILRQRERRK

SEQ ID NO: 17 - protein, 665 aa, Pseudomonas aeruginosa
MLDNLRIKLLGLPRRYKRMLQVAADVTLVWLSLWLAFLVRLGTEDMISPFSGHAWLFIAAPLVAIPLFIRFGMYRAVMRYLGNDALIAIAKAVTISALVLSLLVYWYRSPPAVVPRSLVFNYWWLSMLLIGGLRLAMRQYFMGDWYSAVQSVPFLNRQDGLPRVAIYGAGAAANQLVAALRLGRAMRPVAFIDDDKQIANRVIAGLRVYTAKHIRQMIDETGAQEVLLAIPSATRARRREILESLEPFPLHVRSMPGFMDLTSGRVKVDDLQEVDIADLLGRDSVAPRKELLERCIRGQVVMVTGAGGSIGSELCRQIMSCSPSVLILFEHSEYNLYSIHQELERRIKRESLSVNLLPILGSVRNPERLVDVMRTWKVNTVYHAAAYKHVPIVEHNIAEGVLNNVIGTLHAVQAAVQVGVQNFVLISTDKAVRPTNVMGSTKRLAEMVLQALSNESAPLLFGDRKDVHHVNKTRFTMVRFGNVLGSSGSVIPLFREQIKRGGPVTVTHPSITRYFMTIPEAAQLVIQAGSMGQGGDVFVLDMGPPVKILELAEKMIHLSGLSVRSERSPHGDIAIEFSGLRPGEKLYEELLIGDNVNPTDHPMIMRANEEHLSWEAFKVVLEQLLAAVEKDDYSRVRQLLRETVSGYAPDGEIVDWIYRQRRREP

SEQ ID NO: 18 - protein, 463 aa, Pseudomonas aeruginosa
MINSHLLYRLSYRGTARRMLLIKKGKPLPMTSPFSLQDLDDGLGDGLQVRFVQRGDADTAGADGVDTELGLQALDLVGGQAGIGEHATLATDETEVALGAVGCQLLDHRQAHVADAVAHLAQFLLPEGPQFRAVEHGGDDAGAVGRWVRIVGADHPLHLGQHAGRFIAAFGHDREGADAFAIEREGFGERAGNEEAQARLGEQAHRGGVFLDAVAEALVGDVEERHVALGLEHVQHLFPVVQLEIDAGRIMAAGVQNHDRAGRQGIQVFQQAGAVHAIAGGVVIAVVLHREAGGFEQCAVVFPARVADGHGGVGQQALEEVGAELERAGAADGLGRDHTAGGQQLGLVTEQQFLYALVVGGDPFDRQVAARRVGLDAGLLGSLHGTQQRNAPLLVVVHAHAQVDLARTGIGVEGFVQAKDGITRCHFDGRKQTHFAAARSVKRGGQRNPLCGGAKGCANGGLL

SEQ ID NO: 19 - protein, 238 aa, Pseudomonas aeruginosa
MHAATFRCMLSAISDAGFSLASQLPARFFMDTFQLDSRFKPAGDQPEAIRQMVEGLEAGLSHQTLLGVTGSGKTFSIANVIAQVQRPTLVLAPNKTLAAQLYGEFKTFFPHNSVEYFVSYYDYYQPEAYVPSSDTYIEKDSSINDHIEQMRLSATKALLERPDAIIVATVSSIYGLGDPASYLKMVLHLDRGDRIDQRELLRRLTSLQYTRNDMDFARATFRVRGDVIDIFPAESDLE

SEQ ID NO: 20 - protein, 303 aa, Pseudomonas aeruginosa
MMIWMIACLVVLLFSFVATWGLRRYALATKLMDVPNARSSHSQPTPRGGGVAIVLVFLAALVWMLSAGSISGGWGGAMLGAGSGVALLGFLDDHGHIAARWRLLGHFSAAIWILLWTGGFPPLDVVGHAVDLGWLGHVLAVFYLVWVLNLYNFMDGIDGIASVEAIGVCVGGALIYWLTGHVAMVGIPLLLACAVAGFLIWNFPPARIFMGDAGSGFLGMVIGALAIQAAWTAPSLFWCWLILLGVFIVDATYTLIRRIARGEKFYEAHRSHAYQFASRRYASHLRVTLGVLAINTLWLLRWH

SEQ ID NO: 21 - DNA, 19 bp, E.coli
catttgacatggtataatg

SEQ ID NO: 22 - DNA, 19 bp, Pseudomonas aeruginosa
cttttgtgaacgcagaaag

SEQ ID NO: 23 - DNA, 19 bp, Pseudomonas aeruginosa
tatttgccccgctttgttg

SEQ ID NO: 24 - DNA, 19 bp, Pseudomonas aeruginosa
cacttggcagtcaagattg

SEQ ID NO: 25 - DNA, 19 bp, Pseudomonas aeruginosa
ctgttggcacagtttgctg

SEQ ID NO: 26 - DNA, 19 bp, Pseudomonas aeruginosa
gttttggcgcactaagcag

SEQ ID NO: 27 - DNA, 19 bp, Pseudomonas aeruginosa
tagttgatgaactacctag

SEQ ID NO: 28 - DNA, 19 bp, Pseudomonas aeruginosa
tgcttgctgacggatcgtc

SEQ ID NO: 29 - DNA, 34 bp, Pseudomonas aeruginosa
aaattgaggtgagttggaaaatgatagatgttaa

SEQ ID NO: 30 - DNA, 34 bp, Pseudomonas aeruginosa
tcatttccataggacgaaccatgaaaaatttcgc

SEQ ID NO: 31 - DNA, 34 bp, Pseudomonas aeruginosa
ctttggcaagctgcagcgtaatgttgtgcacttc

SEQ ID NO: 32 - DNA, 34 bp, Pseudomonas aeruginosa
tcgagtgtgagtctcaagccatgagttattatca

SEQ ID NO: 33 - DNA, 34 bp, Pseudomonas aeruginosa
agcaaggtggacgtgtgaccatgattgaattcat

SEQ ID NO: 34 - DNA, 34 bp, Pseudomonas aeruginosa
ctgcgttgacgaattgacggatgtatatatactt

SEQ ID NO: 35 - DNA, 34 bp, Pseudomonas aeruginosa
atgtctttaggaaaaactctatgagtgcggcttt

SEQ ID NO: 36 - DNA, 34 bp, Pseudomonas aeruginosa
tgtgccaagggagatgccaagtgatcgttgttat

SEQ ID NO: 37 - DNA, 34 bp, Pseudomonas aeruginosa
aacttcgtggagcttgtctgatggtccggaggcg

SEQ ID NO: 38 - DNA, 34 bp, Pseudomonas aeruginosa
tgcttcgggaggttgttgtgatgaaagatctgtt

SEQ ID NO: 39 - DNA, 34 bp, Pseudomonas aeruginosa
cgtgatgaccggggccgctcatgactaaagttgc

SEQ ID NO: 40 - DNA, 34 bp, Pseudomonas aeruginosa
ctgagtaagcgagattccatatgaaaattctgac

SEQ ID NO: 41 - DNA, 34 bp, Pseudomonas aeruginosa
taaaggatttatttagttccatgaacgtctggta

SEQ ID NO: 42 - DNA, 34 bp, Pseudomonas aeruginosa
cttgctgatgggcgcgcagcatgaaagctgtcat

SEQ ID NO: 43 - DNA, 34 bp, Pseudomonas aeruginosa
gaacggggctgataaataggatgttggataattt

SEQ ID NO: 44 - DNA, 34 bp, Pseudomonas aeruginosa
ggactcgaaccagggacccaatgattaacagtca

SEQ ID NO: 45 - protein, 29 aa, Pseudomonas aeruginosa
LIGIVGLGYVGLPLMLRYNAIGGDVLGID

SEQ ID NO: 46 - protein, 35 aa, Pseudomonas aeruginosa
AVMVTGASGFVGSALCCELARTGYAVIAVVRRVVE

SEQ ID NO: 47 - protein, 31 aa, Pseudomonas aeruginosa
VVMVTGAGGSIGSELCRQIMSCSPSVLILFE

SEQ ID NO: 48 - protein, 30 aa, Pseudomonas aeruginosa
LVIQAGSMGQGGDVFVLDMGPPVKILELAE

SEQ ID NO: 49 - protein, 29 aa, Pseudomonas aeruginosa
RISIFGLGYVGAVCAGCLSARGGEVIGVD

SEQ ID NO: 50 - protein, 31 aa, Bordetella pertussis
VVMVTGAGGSIGSELCRQILALRPRKLVLFE

SEQ ID NO: 51 - protein, 30 aa, Bordetella pertussis
LVLQAGAMGESGSVFVLDMGEPVLIRELAE

SEQ ID NO: 52 - protein, 34 aa, Staphylococcus aureus
TILVTGAGGSIGSEICRQVSKFDPQKIILLGHGE

SEQ ID NO: 53 - protein, 30 aa, Staphylococcus aureus
KILITGTAGFIGSHLAKKLIKQGGYVIGVD

SEQ ID NO: 54 - protein, 28 aa, Staphylococcus aureus
NIAVVGLGYVGLPVAVTFGNKHKVIGFD

SEQ ID NO: 55 - protein, 30 aa, Nocardia sp.
CVLVTGGSGFVGANLVTELLDRGYAVRSFD

SEQ ID NO: 56 - protein, 29 aa, Pseudomonas Solanacearum
TISVVGLGYIGLPTATVLASRQRELIGVD

SEQ ID NO: 57 - protein, 30 aa, Rhizobium meliloti
NILVVGGAGYIGSHTCLQLAADGYQPVVYD

SEQ ID NO: 58 - protein, 30 aa, E.coli
RVLVTGGSGVIGSKTCVQLLQNGHDVIILD

SEQ ID NO: 59 - protein, 36 aa, Streptomyces violaceoruber
RLLVTGAAGFIGSHYVREILAGSYPESDDVHVTVVD

SEQ ID NO: 60 - protein, 31 aa, E.coli
KILITGGAGFIGSALVRYIINETSDAVVVVD

SEQ ID NO: 61 - protein, 28 aa, Shigella sonnei
KIGIIGLGYVGLPLAVEFGKKVTTIGFD

SEQ ID NO: 62 - protein, 28 aa, E.coli
KIAIIGLGYVGLPLAAEFGKIRQVVGFD

SEQ ID NO: 63 - protein, 35 aa, Vibrio cholerae
VYLIYGAGSAGRQLAIALRNSENYKEVIMGMQVHD

SEQ ID NO: 64 - protein, 31 aa, E.coli
KILVTGGAGFIGSAVVRHIINNTQDSVVNVD

SEQ ID NO: 65 - protein, 29 aa, E.coli
TISVIGLGYIGLPTAAAFASRQKQVIGVD

SEQ ID NO: 66 - protein, 30 aa, Streptomyces glauciens
RILLTGHQGYLGTVMAPVLTAAGHQVTGLD

SEQ ID NO: 67 - protein, 31 aa, Yersinia enterocolitica
VVMVTGAGGSIGSELCRQIIVEKPSLLILFD

SEQ ID NO: 68 - protein, 27 aa, Yersinia enterocolitica
LVIQAGAMGQGGDVFVLDMGDPVKIID

SEQ ID NO: 69 - protein, 29 aa, Staphylococcus aureus
RVAIFGTGYVGLVTGTCLAEVGHHVICVD

SEQ ID NO: 70 - protein, 28 aa, Salmonella typhi
KIAIIGLGYVGLPLAVEFGKSRQVVGFD

SEQ ID NO: 71 - protein, 30 aa, Salmonella typhi
RWLITGVAGFIGSGLLEELLFLNQTVIGLD

SEQ ID NO: 72 - protein, 436 aa, Pseudomonas aeruginosa; identical to SEQ ID NO: 3

SEQ ID NO: 73 - protein, 420 aa, E.coli
MSFATISVIGLGYIGLPTAAAFASRQKQVIGVDINQHAVDTINRGEIHIVEPDLASVVKTAVEGGFLRASTTPVEADAWLIAVPTPFKGDHEPDMTYVESAARSIAPVLKKGALVILESTSPVGSTEKMAEWLAEMRPDLTFPQQVGEQADVNIAYCPERVLPGQVMVELIKNDRVIGGMTPVCSARASELYKIFLEGECVVTNSRTAEMCKLTENSFRDVNIAFANELSLICADQGINVWELIRLANRHPRVNILQPGPGVGGHCIAVDPWFIVAQNPQQARLIRTAREVNDHKPFWVIDQVKAAVADCLAATDKRASELKIACFGLAFKPNIDDLRESPAMEIAELIAQWHSGETLVVEPNIHQLPKKLTGLCTLAQLDEALATADVLVMLVDHSQFKVINGDNVHQQYVVDAKGVWR

SEQ ID NO: 74 - protein, 423 aa, B. solanaceraeum
MDRAIEIDFRTISVVGLGYIGLPTATVLASRQRELIGVDINQHAVDTINQARIHIVEPDLDMLVRAAVSQGYLRATTEPEPADAFLIAVPTPFLEDKQPDLTYIEAAAKAIAPVLKRGDLVVLESTSPVGATEQLSAWLSEQRSDLSFPHQLGEESDIRVAHCPERVLPGHVLRELVENDRIIGGMTPRCSQAAQRLYELFVRGRCIVTDARTAEMCKLTENAFRDVNIAFANELSMICDEIGVNVWELISVANRHPRVNILQPGPGVGGHCIAVDPWFIVDAAPESARLIRTAREVNDAKPHYVLDRVKQAARRFKEPVIACFGLSFKANIDDLRESPAIEIVRTMVQQQLGTVLVVEPHIKVLPASLEGVELLNAEPALSRADIVVLLVDHQKFRKLDTDRLQSRVVIDTRGMWSAKRLAA

SEQ ID NO: 75 - protein, 163 aa, Pseudomonas aeruginosa
MSYYQHPSAIVDDGAQIGSDSRVWHFVHICAGARIGAGVSLGQNVFVGNKVVIGDRCKIQNNVSVYDNVTLEEGVFCGPSMVFTNVYNPRSLIERKDQYRNTLVKKGATLGANCTIVCGVTIGEYAFLGAGAVINKNVPSYALMVGVPARQIGWIANSVSSCS

SEQ ID NO: 76 - protein, 191 aa, Bordetella pertussis
MTTIHPTAIVDEGARIGANSRIWHWVHICGGAEIGAGCSLGQNVFVGNRVRIGDRVKIQNNVSVYDNVFLEDDVFCGPSMVFTNVYNPRAAIERKNEYRDTLVRQGATLGANCTIVCGATVGRYAFVGAGAVVNKDVPDFALVVGVPARQIGWMSRHGEQLDLPLAGNGQARCPHTGDLYILENGVCRLGE

SEQ ID NO: 77 - protein, 276 aa, Pseudomonas aeruginosa; identical to SEQ ID NO: 7

SEQ ID NO: 78 - protein, 366 aa, Bordetella pertussis
MQFIDLKTQYQALRDTINPRIQAVLDHGQFIMGPEVKELEAALCAYTGAKHCITVASGTEALLISLMALGVKAGDEVITTSFTFVATAEVIALLGAKPVFVDVEPDTCNIKVSEIEAKITPRTKAIIPVSLYGQCGDMDEVNAVAARHGLPVIEDAAQSFGATYKGRKSCNLSTIGCTSFFPSKPLGCYGDGGALFTNDDELAQAMREIRVHGQSGRYYHARIGVGGRMDTLQCAVVLGKLERFDWEIAQRIKIGARYQQLLADLPGGACTVTVRPDRDSVWAQFTVMVPNREAVIAQLKEAGIPTAVHYPRPIHAQPAYEQYAEGAGATPVSDDLAARVMSLPMHPDLDEATQDKIVAALRQALN

SEQ ID NO: 79 - protein, 372 aa, Bacillus subtilis
MNVPMLDLSEQYEQLKPEIMRVLDEVMRSSRFILGDYVKKLEADIAAYSRAKHGIGCGNGSDAIHIALQAAGVGPGDEVITTAFTFFATAGSIARAGAKPVFVDIDPVTFNIDPAQVEAAVTEKTKAIIPVHLYGQMADMEAIAAIAKRHGLVVIEDAAQAIGAKYNGKCVGELGTAATYSFFPTKNLGAYGDGGMIITNDDELAEKCRVIRVHGSKPKYYHHVLGYNSRLDEMQAAILSVKFPHLDRWTEQRRKHAATYTRLLEEAVGDLVVTPKEVDGRYHVFHQYTIRAPKRDELQAFLKEQGIATMVYYPLPLHLQPVFASLGYKEGQLPEAEKAAKEALSLPMFPELKEEQQQYVVEKIAEFYRHFA

SEQ ID NO: 80 - protein, 365 aa, Saccharopolyspora erythraea
MDVPFLDLQAAYLELRSDIDQACRRVLGSGWYLHGPENEAFEAEFAAYCENAHCVTVGSGCDALELSLVALGVGQGDEVIVPSHTFIATWLGVPVGAVPVPVEPEGVSHTLDPALVEQAITPRTAAILPVHLYGHPADLDALRAIADRHGLALVEDVAQAVGARHRGHRVGAGSNAAAFSFYPGKNLGALGDGGAVVTTDPALAERIRLLRNYGSKQKYVHEVRGTNARLDELQAAVLRVKLRHLDDWNARRTTLAQHYQTELKDVPGITLPETHPWADSAWHLFVLRCENRDHLQRHLTDAGVQTLIHYPTPVHLSPAYADLGLPPGSFPVAESLAGEVLSLPIGPHLSREAADHVIATLKAGA

SEQ ID NO: 81 - protein, 370 aa, Streptomyces peucetius
MSTYVWQYLNEYREERADILDAVETVFESGQLILGTSVRSFEEEFAAYHGLPYCTGVDNGTNALVLGLRALGIGPGDEVVTVSNTAAPTVVAIDAVGATPVFVDVHEENYLMDTGRLRSVIGPRTRCLLPVHLYGQSVDMTPVLELAAEHDLKVLEDCAQAHGARRHGRLVGTQGHAAAFSFYPTKVLGAYGDGGAVVTPDAEVDRRLRRLRYYGMGERYYVVDTPGHNSRLDEVQAEILRRKLRRLDAYVEGRRAVARRYEEGLGDLDGLVLPTIAEGNDHVYYVYVVRHPERDRILEALTAYDIHLNISYPWPVHTMSGFAHLGYGPGDLPVTERLAGEIFSLPMYPSLRPDAQEKVIDAVREVVGSL

SEQ ID NO: 82 - protein, 389 aa, Bacillus subtilis
MVQKRNHFLPYSLPLIGKEEIQEVTETLESGWLSKGPKVQQFEKEFAAFVGAKHAVAVNSCTAALFLALKAKGIGPGDEVITSPLTFSSTANTIIHTGATPVFADIDENTLNIDPVKLEAAVTPRTKAVVPVHFGGQSCDMDAILAVAQNHGLFVLEDAAHAVYTTYKQRMIGSIGDATAFSFYATKNLATGEGGMLTTDDEELADKIRVLSLHGMSKAAWNRYSSNGSWYYEVESPGYKMNMFDLQAALGLHQLKRLDDMQKRREEIAGRYQTAFQQIPGLITPFVHDDGRHAWHLYVLQVDEKKAGVTRSEMITALKDEYNIGTSVHFIPVHIHPYYQKQFGYKEADFPNAMNYYKRTLSLPLYPSMSDDDVDDVIEAVRDIVKGAD

SEQ ID NO: 83 - protein, 362 aa, Pseudomonas aeruginosa; identical to SEQ ID NO: 14

SEQ ID NO: 84 - protein, 362 aa, Bordetella pertussis
MPKKILTVLGARPQFIKASVVSAAIAQHPELTEVVVHTGQHFDANMSDVFFDELGMQTPAHQLDIHGGGHGDMTGRMLVALEQVMQAEKPDVVLVYGDTNSTLAGALAAVKLHIPVAHVEAGLRSFNLRMPEEVNRILTDRISRWLFTPTDSATRHLAAEGQSGEKVVQVGDVMYDVALHHGARVTAEGRALAAHGLKPGGYVLATIHRAENTDDAQRLTTIVRALQALAAERQVVWPLHPRTWGILARLGLLDELASTVTLLEPVGYLDMVQLEKYAALIATDSGGVQKEAFFHRIPCVTLRDETEWTELVDAGWNRLAPPVSSAVVAQAVQDALREQPRDVQPYGDGQAARRIVDALAAH

SEQ ID NO: 85 - protein, 376 aa, E.coli
MKVLTVFGTRPEAIKMAPLVHALAKDPFFEAKVCVTAQHREMLDQVLKLFSIVPDYDLNIMQPGQGLTEITCRILEGLKPILAEFKPDVVLVHGDTTTTLATSLAAFYQRIPVGHVEAGLRTGDLYSPWPEEANRTLTGHLAMYHFSPTETSRQNLLRENVADSRIFITGNTVIDALLWVRDQVMSSDKLRSELAANYPFIDPDKKMILVTGHRRESFGRGFEEICHALADIATTHQDIQIVYPVHLNPNVREPVNRILGHVKNVILIDPQEYLPFVWLMNHAWLILTDSGGIQEEAPSLGKPVLVMRDTTERPEAVTAGTVRLVGTDKQRIVEEVTRLLKDENEYQAMSRAHNPYGDGQACSRILEALKNNRISL

SEQ ID NO: 86 - protein, 380 aa, Bacillus subtilis
MKKLKVMTVFGTRPEAIKMAPLVLELKKYPEIDSYVTVTAQHRQMLDQVLDAFHIKPDFDLNIMKERQTLAEITSNALVRLDELFKDIKPDIVLVHGDTTTTFAGSLAAFYHQIAVGHVEAGLRTGNKYSPFPEELNRQMTGAIADLHFAPTGQAKDNLLKENKKADSIFVTGNTAIDALNTTVRDGYSHPVLDQVGEDKMILLTAHRRENLGEPMENMFKAIRRIVGEFEDVQVVYPVHLNPVVREAAHKHFGDSDRVHLIEPLEVIDFHNFAAKSHFILTDSGGVQEEAPSLGKPVLVLRDTTERPEGVEAGTLKLAGTDEENIYQLAKQLLTDPDEYKKMSQASNPYGDGEASRRIVEELLFHYGYRKEQPDSFTGK

SEQ ID NO: 87 - protein, 378 aa, Salmonella enterica
MSKVLFVFGTRPEAIKMAPLVIEFKNNPAIEVKVCVTGQHREMLDQVLDFFEIEPDYDLNIMKQKQSLGSITCSILTRLDEILASFMPAHIFVHGDTTTTFAASLAAFYQNIKVWHIEAGLRTWNMNSPFPEEGNRQLTSKLAFFHAAPTLQAKDNLLRESVKEKNIIVTGNTVIDALLIGIKKITGSTGDVREIISLKNKLNLDKKIILVTLHRRENQGELLRTICDDIKQLALEHDDIEIVFPVHMSPRIREVVNEKLSGVVNIKLVEPLAYPGFIWLMNNAHFILSDSGGVQEEAPSLQKPVLVARDTTERPEVIENGAAMLVDPRIPNNIYSSCKKLLSDERLYEKMSQAGNPFGDGKASKKILDYFVSLEDIK

SEQ ID NO: 88 - protein, 413 aa, Pseudomonas aeruginosa; identical to SEQ ID NO: 15

SEQ ID NO: 89 - protein, 403 aa, Bordetella pertussis
MEFRPYYFGREWIGHGHQVKVAASTISHIRARAPQAGGRLTRENVDGIEYLWYATLPYQGNGARRLLNMLQFSARLYGLRRDLGGWRPDIVIASSTHPYDVLPAARLARQTGARLVFEVHDLWPLTPRLLGGFKAWHPMIASMQYAEDYAYRHADLTVSMLPCALPYMRERGLDPRRYAHVPNGVPVTEYSSPDFDNPDYLRVRAQIRQLREQCDFVLAYAGTHGHANALDMLLQAMARLRDQPIGLLLLGDGPDKPELKRLAGQLGLRHIAFADPVPRPAVQAVMADIDAAYIGLRRSPLFQFGVSPNKLFDYMLSACPVVQSIESGNDIVADARCGLSVPAEDPAALAAALHGLRTLPAAERQAMGRRGRDYVLARHDYPVLAQQFLDAVQSVTPRRAASR

SEQ ID NO: 90 - protein, 344 aa, Yersinia enterocolitica
MYEAGHNVMIISLTGETLVRPNDGIQLNELKLDKAPFSLFKGLFEVKKIIKKFKPDIVHSHMFHANLFARILRVFTKIPALICTAHNTNEGSSLRMLAYKYTDKLASLSTNVSQDAVDSFIHKGASSTGRMIAVSNGIDASQFDFSMDERKVKRSELGIFNDTPIILSVGRLTEAKDYPNLLTAFSLLIKDNSLQSFPQLFIVGTGHLDGYLKNMSKEFGIDKYVTLFGQRDDILQLMCAADIFVLSSEWEGFPLVITEAMACKKIIVATDAGGITEALGDCGSIVPIKDPNSLSQAINKMIKLSDNEKEILGNKARERIIQTNSIEKIIELGCLFILNLKNNC

SEQ ID NO: 91 - protein, 303 aa, Pseudomonas aeruginosa; identical to SEQ ID NO: 20

SEQ ID NO: 92 - protein, 341 aa, Yersinia enterocolitica
MPTFFFLLTIFFLLSVGLTYLLRLYALKNNIIDTPNSRSSHVTPTPRGGGVAIVISFLIGIILFYFLGYLPILSVVGLIVSGGVIALVGFWDDHGHIAARWRLLAHFSAAAFLLFCFGGFPVLNVSGFIIELGIFGSLFGLLFLVWMLNLYNFMDGIDGLASAEAVTACIGMIAIYYISGDHIELNSFLVLWLLACTVLGFLLWNFPPAKIFMGDAGSGFLGLMIGSLAISAGWIDTRFFFCWLILLGLFIVDATWTLVRRVLGGFKVYEAHRSHGYQIASRRFKRHLPVTLSAIAINIIWLFPIALLAGLNIVNPIIALIISYIPLLYIDYKLNAGVNND

SEQ ID NO: 93 - protein, 355 aa, E.coli
MLSIFVTFLGAFLTLIVMRPLANWIGLVDKPNYRKRHQGTIPLIGGASLFVGNLCYYLMEWDQLRLPYLYLFSIFVLLAIGILDDRFDISPFLRAGIQAILAILMIDLGNIYLDHLGQILGPFQLTLGSIGLIITVFATIAIINAFNMIDGIDGLLGGLSCVSFAAIGILMYRDGQMDMAHWSFALIVSILPYLMLNLGIPFGPKYKVFMGDAGSTLIGFTIIWILLLSTQGKGHPMNPVTALWIIAIPLIDMVAIIYRRVRKGKSPFRPDRLHVHHLMVRAGLTSRQAFLLITFVSAVCATIGILGEVYYVNEWAMFVGFFILFFLYVYSITHAWRITRWVRRMKRRAKRLKKA

SEQ ID NO: 94 - protein, 665 aa, Pseudomonas aeruginosa; identical to SEQ ID NO: 17

SEQ ID NO: 95 - protein, 638 aa, Yersinia enterocolitica
MFLVFLLSLPRPVKRTIMLLLDTILIALAYWGAFWVRLDVDSPFTSIEQWVALAAIIPPTLFAYIKLGLYRTVLRYVSAKIVSIVLVGVVLSSGLLVLGSYFLGVYLPRTVSVMFFIFSLVLICGSRLFFRMLLNYGVRGQIPVVIYGAGASGRQLLPALMQASEYFPIAFVDDNPKLHKAVIHGVTVYPSEKLEYLIGRYGIKKVLLAMPSVSQSQRRAVVNKLENLSCEVLSIPGMSDLVEGRAQISSLKKVSIEELLGRDPVVPDEKLLAKNITGKVVMVTGAGGSIGSELCRQIIVEKPSLLILFDISEFSLYSIENEMAAICKKNKIETEFVALLGSVQSEKRLVQIMSNFHVNTVYHAAAYKHVPLVENNVIEGVRNNIFGTLYCAKAAIKSGVEKFVLISTDKAVRPTNTMGATKRMAELVLQALSTEQNKTKFCMVRFGNVLGSSGSVVPLFKKQIAEGGPITLTHKDIIRYFMTIPEAAQLVIQAGAMGQGGDVFVLDMGDPVKIIDLAKRMINLSGLSIKSEENLDGDIAIEISGLRPGEKLYEELLIGDSVQHTYHPRIMTATEIMLEWDDLNILLNKIETACNDFNYECIRSLLLEAPTGFQPTDGICDVVWQKTHSENAKNVIVH

SEQ ID NO: 96 - protein, 624 aa, Bordetella pertussis
MTLPYAIRRLFVDLPRPFKQMLAIVLDAVILLGAFHLALWLRFELFFLTDQYLFLSLLACAGGIAALAAFGVYLYILRYMSERVLAAILGGIVVSVMVVTAGNTFLQLATISRGVLVLYAALALVGLIGVRLIARKLLFPADHHMADPRTPVLIYGAGGAGSQLAMALRTGPHYRPVAMLDDDKRKHRLVVNGLRVYPPEQLPKLIDRHNIRQLLIAMPSAPPKQIRSIVEAAEPYRLRIRLVPSMRELIDPTNGVRLRDVQVEDLLGRDPVAPIDTLLGRCVTDRVVMVTGAGGSIGSELCRQILALRPRKLVLFEIAEPALYAIEQDLRQRIGERNIEIAGVLGSVRDAAHCLAQLQEHGVQTIYHAAAYKHVPIVEHNVSEGIRTNAFGTLNMAETAIQAGVLDFVLISTDKAVRPTNVMGASKRLAELILQAHAQIQDKTRFSMVRFGNVLGSSGSVVPLFRRQILEGGPITLTHPEITRYFMTIPEAAQLVLQAGAMGESGSVFVLDMGEPVLIRELAERMVRLYGLTVKNSDQPDGDIEIRITGLRPGEKLYEELLIGEDSRETLHPRIMRATEYSLPYETLMGQLRMLDRSLQMCSPRQAAELLGQIVREYASVTYA

SEQ ID NO: 97 - protein, 599 aa, Staphylococcus aureus
MTSISAKLRFLILIIIDSFIVTFSVFLGYAILEPYFKGYSIDLLVLSSVILLVSHHIFAYVFNLYHRAWEYASVSELMSVLKAVTSSIVVTLLLVSLLISESPFLRLYFITWMMHLLLIGGSRLFWRVYRRYFIDNAVEKKATLVVGAGQGGSVLIREMLRSQDMRMQPVLAVDDDKNKQKMTITERVKVQGYVEDIPELVKKFRIKKIIIAIPTLSQKRLNEINKICNIEGVELFKMPNIEDVLSGELEVNNLKKVEVEDLLGRDPVELDMALISRELTNKTILVTGAGGSIGSEICRQVSKFDPQKIILLGHGENSIYSIHQELSKTYGNRIEFVPVIADVQNKTRILEVMNEFKPYAVYHAAAHKHVPLMEYNPHEAIRNNILGTKNVAESAKEGEVSKFVMISTDKAVNPSNVMGATKRIAEMVIQSLNEDNSKTSFVAVRFGNVLGSRGSVIPLFKNQIESGGPVTVTHPEMTRYFMTIPEASRLVLQAGALAQGGEVFVLDMGKPVKIVDLAKNLIRLSGKKEEDIGIEFSGIRPGEKLYEELLNKNEIHPQQVYEKIYRGKVDHYIKTEVDLIVEDLINNFSKEKLLKIANR

SEQ ID NO: 98 - DNA, 1047 bp, Pseudomonas aeruginosa
atgactgacgaaatacaaaagcacggcggtgtagctggcgatatcgatctggttgagctggttcgaggattatgggaggagaagtggatagttcttatattttctttgctaggtattttgtttgcagctatctacgcttttctcagtactcctgtctatgaggcccgcatagcgattttgcctccgtcgttgagtgatgtggcaggtttcaatcagggacgtaccagggaaaccgggcttggtcccttcaaggtccaggatgtgtactctgtttttgttcgcaacctgcaggctgatggaactcgtcatcgttttttcaatgagacctatttgccttctttggatgaagagcttcgttcggtttcgcgtgatgcgctctataaaaggttcactgatcagataagtattagtttgccggggaaagactttccgggtcgttatcttgttgcgattgaacaggaggatccggagcgtgcggcgagttgggttcgtcggtatatagctgatgcggccgagatttctattcaggaaatgttgaacaatgcgcatcgcgagattgaggtcaaggctcgagatattgagcagcgcatacagaacttgcggagagaatgccaaggcagacgtgaagatcgtattgttcagctcaaggaggcgttgaaggtcgcaggtgcgctgaaattggaggagcctccactgatcagtgggcaatcctctgaggagctctcggctatcatgaatggaagtctgatgtatatgcgtggcagtaaggcgattatggccgagattcagacattggaggcgcgtagctctgatgatccttttattccggcgttgcgtactcttcaggagcagcagttattgctgagtagcttgcgtgttaattcggagcgggtttctgtttttcgacaagacggtccgatagaaacgccggactcaccagttcgtccaaggagagcgatgattttgatttttgggttgataattggtggtgtgcttggtggttttctggcgttgtgccggatttttttgaagaagtatgctcgttag

SEQ ID NO: 99 - DNA, 920 bp, Pseudomonas aeruginosa
gagctcgagttcaaggtcatcaagctcgaccagaagcgcaacaacgttgtcgtttcccgccgcagcgtccggaagccgagaacagcgccgagcgtgaagctctgctggaatcgctgcaggaaggccagcaggtcaaaggttcgtcaagaacctcaccgactacggcgcattcgtggacctgggcggcgtagacggcctgctacacatcacgacatggcctggaagcgcatcaagcatccgtccgagatcgtcaacgttggcgacgagatcgacgtcaaggcctgaagttcgaccgcgagcgcaaccgtgtatccctgggcctgaagcaactgggcgaagacccgtgggttccatcaaggcgcgttacccggaagtaccgcgtcatggccgcgtcaccaacctcaccgactacggctgcttgccgaactggaagaggcgtggaagctggtacacgtctccgaaatggactggaccaacaagaacatccatcgtcgaaagtcgtccaggttggcgatgaagtggaagttcaggttctggacatcgacgaagagcgtcgtcgttctccctgggtatcaagcagtgcaaatccaacccgtgggaagacttctccagccagttcaacaagggtgacgtatctccggtaccatcaagtcgatcaccgacttcggtatcttcatcggtctggacggcggcatcgacgcctggtccacctgtccgacatctcctggaacgaagtcggcgaagaagccgtacgtcgcttcaagaagggcacgagctggaaaccgtcatcctgtcggtcgatccggagcgcgagcgcatctccctgggcatcaagcagctgaagacgatccgttctccaactacgcgtcctgcacgagaaagcagcatcgtccgcggtaccgtgaaggaatcaacgccca

SEQ ID NO: 100 - DNA, 1217 bp, Pseudomonas aeruginosa
aaaaatcgaagtatcctgaaggcttccgaaatcagccgtgaccgcgtcgaagacgcgcgcaagtcctgaagaaggggaggaagtcgaagccaagattatcagcatcgaccgcaagagcgggtcatcagcttttccgtcaatccaaggacgtcgacgacgagaaggacgcaatgaaagaactgcgtaagcaggaagtagaaagcgctggtcgaccaccatcggtgatctgatccgtgctcagatggagaatcagggctaagtctctgatccatcatgaaaagggcggcctaggccgccctttttcgttttccccttcttggacctgttcaaagactgatcagcatgctaaagagacctgagctgatctagccgcttgaaaaagaagggaaaaccatgaccaagtcggagttgatcgaacggtcgttacccatcaggggcaactgtccgcgaaggatgtcgagttggcaatcaagaccatgctggagcaaattcccaggccctggcgaccgggaccggatcgagatccgtggcttcggcagcttttccttgcattaccgcgcccgcgcgtcgttcgcaaccccaagaccggggagtcggtacgcttcgacggcaagttcgtgccgcacttcagccgggcaaggagttgcgggatcgggtcaacgagccggagtgatttctgccttgttcagatgttggagttccatgctttgggtcaagcgtacgttaatggcggtggggctgttagttgtcgcccttttcatgattgtggtgctttggagaaccggcaaagcgtcagctttgaactctttggtcttgccacgccagatttacctgtggtccttatgttgcgttagcatttattgctggcggtattattggtatgttgatcagcgtgcctcttctggctcgtccaaagtgcgtctcagatctgcaagatctgatctgtttcgtactcgaaaagaactcgcagtatctcagtcaccgccctgcggtgaggtctgctcgagtccctgcctgttctttgtgggctcgagtgctattcgcatctagtgacaacacaatgcttggaaggtcgggtgggatgggtgtcctgttagagggggtgctgagttaccatgtctactggtttggctggagtctgtagatggagtctgtagatggaggcttggttcatggcatcgtgtcgctccggttggtcttcgccaaaggtcaagctt